(19) United States
(12) Patent Application Publication
(10) Pub. No.: US 2010/0130581 A1
(43) Pub. Date: May 27, 2010

(54) COMPOUNDS AND METHODS FOR THE TREATMENT OF PAIN

(75) Inventors: Istvan Toth, Brookfield (AU); Yasuko Koda, North Sydney (AU); Mark Pasqualino Del Borgo, Moorabbin (AU); Joanne Therese Blanchfield, Upper Mount Gravatt (AU)

Correspondence Address: FOLEY AND LARDNER LLP;SUITE 500, 3000 K STREET NW, WASHINGTON, DC, 20007, US

(73) Assignee: THE UNIVERSITY OF QUEENSLAND

(21) Appl. No.: 12/295,187

(22) PCT Filed: Mar. 30, 2007
(86) PCT No.: PCT/AU2007/000419

§ 371 Date: Oct. 5, 2009

Related U.S. Application Data
(60) Provisional application No. 60/788,530, filed on Mar. 31, 2006.

(51) Int. Cl. A61K 31/403; C07D 401/12

(52) U.S. Cl. 514/414; 548/467

(57) ABSTRACT

This invention relates to derivatives of an endomorphin, or of an endomorphin analog, comprising at least one moiety selected from a lipid moiety, a cyclitol moiety and a saccharide moiety.

### FIELD OF THE INVENTION

This invention relates generally to compounds and methods useful for modulating opioid receptors. In particular, the invention relates to compounds and methods useful for modulating μ-opioid receptors (MOR) in the treatment, prophylaxis, reversal and/or symptomatic relief of pain.

### BACKGROUND OF THE INVENTION

Centrally acting opiates, such as morphine, are the most frequently used analgesics for the relief of severe pain, although they are known to bring about a number of well known side effects, including tolerance, physical dependence, respiratory depression, and adverse gastro-intestinal effects. Endomorphins are potent and selective endogenous ligands for the μ-opioid receptor which have been isolated from the human brain cortex. Both morphine and endomorphins act as agonists at the same μ-opioid receptor MOR, but the latter are believed to inhibit pain without some of the undesirable side effects of morphine.

Despite having similar physiological effects to other endogenous opioid ligands, the amino acid sequences of endomorphins are quite distinct from those of the “typical” endogenous opioids such as enkephalins, endorphins and dynorphins. In particular, most endogeneous opioid peptides commonly contain the Tyr¹-Gly² N-terminal sequence, whereas in endomorphin tetrapeptides, the second amino acid is proline instead of glycine.

The C-termini of endomorphins are amidated. C-terminal amidation has been implicated in the efficient binding of the endomorphin tetrapeptides to MOR. Further, the presence of the proline residue at position 2 would also appear to influence the conformation of peptide chains, and may confer stability against many proteases. There are two endomorphin tetrapeptides—namely endomorphin-1 and endomorphin-2. Endomorphin-1 consists of the sequence Tyr-Pro-Trp-Phe-NH₂, whilst endomorphin-2 consists of Tyr-Pro-Phe-Phe-NH₂.

The strong analgesic activity of endomorphins affords the possibility of developing a novel class of painkillers based on their structure. Exogenous application of native opioid peptides, however, is generally not successful on account of their biological instability. For example, endomorphins may be easily degraded by intra- and extracellular peptidases. Enzymes involved in endomorphin metabolism are detailed in: Mentlein, R.; Lucius, R. Methods for the investigation of neuropeptide catabolism and stability in vitro. Brain Research Protocols 1997, 1, 237-246; and Peter, A.; Toth, G.; Tomboly, C.; Laus, G.; Tourwe, D. Liquid chromatographic study of the enzymatic degradation of endomorphins, with identification by electrospray ionization mass spectrometry. Journal of Chromatography A 1999, 846, 39-48.

Moreover, a further hindrance to the physiological efficacy of drugs based on peptides, including endomorphins, is the delivery of the active moiety to the desired point of physiological action. For example, it is well established that one of the principal hindrances to drug uptake, including peptide drug uptake, into central nervous tissue is the blood—brain barrier (BBB).

### SUMMARY OF THE INVENTION

The present invention is predicated in part on the synthesis of a series of lipo-, glyco- and glycolipid derivatives of endomorphin, and of endomorphin analogs, which bind to opioid receptors and which have markedly improved cell permeability and/or stability.

The present invention provides derivatives of endomorphin, and of endomorphin analogs, that allow for improved stability and/or improved passage across a membrane such as the gastro-intestinal (GI) tract, sub-cutaneous (s.c.) layer and/or BBB. In some aspects, the present invention provides derivatives that may display facile GI absorption or crossing of the s.c. layer, but may not cross the BBB.

Thus, in one aspect, the present invention provides lipo-, glyco- and glycolipid derivatives of endomorphin, and of endomorphin analogs. Said derivatives comprise at least one moiety selected from a lipid moiety and a saccharide moiety. Suitably, the at least one moiety is conjugated to the N-terminus, C-terminus, backbone or is a constituent of a side chain of the endomorphin or endomorphin analog.

In some embodiments, the derivative comprises at least one lipid moiety. In others, the derivative comprises at least one saccharide moiety. In still other embodiments, the derivative comprises at least one saccharide moiety and at least one lipid moiety.

In several embodiments, the improved stability and/or improved passage across membranes such as the GI tract, s.c. layer and/or BBB of derivatives of endomorphin, and of endomorphin analogs, that are provided by the present invention, may be attenuated by the inclusion of the at least one moiety selected from a lipid moiety and a saccharide moiety.

In one aspect, the invention provides derivatives of endomorphin, and of endomorphin analogs, comprising a moiety represented by formula I:

Q¹-P¹-Q³-Q⁴ formula I
wherein:
   Q¹ is selected from an optionally substituted phenolic amino acid residue;
   P¹ is an amino acid residue or is a linker moiety which is further substituted with a cyclitol, saccharide moiety and/or a lipidic group;
   Q³ is selected from an optionally substituted aromatic amino acid residue; and
   Q⁴ is selected from an optionally substituted aromatic amino acid residue.
with the proviso that at least one lipidic, cyclitol or saccharide moiety is conjugated to the compound comprising the moiety represented by formula I.

In a further aspect, the present invention provides a method of modulating an opioid receptor comprising contacting an opioid receptor with a lipo-, glycol- or glycolipo-derivative as broadly described above.

In another aspect, the present invention provides a method of delivering an endomorphin, or an endomorphin analog, to neural tissue in a subject, comprising administering to the subject a lipo-, glycol- or glycolipo-derivative as broadly described above.

In another aspect, the invention provides methods for preventing or attenuating pain in a subject, comprising administering to the subject an effective amount of a lipo-, glycol- or glycolipo-derivative as broadly described above, which is suitably in the form of a composition comprising a pharmaceutically acceptable carrier and/or diluent.

In another aspect, the present invention contemplates the use of a lipo-, glycol- or glycolipo-derivative as broadly described above in the manufacture of a medicament for producing analgesia in a subject.

### BRIEF DESCRIPTION OF THE DRAWINGS

FIG. 1(a) is a graphical representation illustrating the ability of native endomorphin-1 (Endo1) (dashed line) and C8-endo-1 (1) (full line) to displace ³H-DAMGO from the μ-opioid receptor. Both show similar Kᵢ values in the low nanomolar range.

FIG. 1(b) is a graphical representation showing the ability of native Endo1 (dashed line) and analog 7 (full line) to displace ³H-DAMGO from the μ-opioid receptor. Analog 7 shows high binding affinity in the picomolar range.

FIG. 2(a) is a graphical representation showing the concentration-response curves of the cAMP inhibition shown by Endo1, C8-endo1 (1) and C8(Dmt)-Endo1 (7) in forskolin treated cells.

FIG. 2(b) is a graphical representation showing inhibition of forskolin stimulated cAMP in SH-SY5Y cells by Endo1 compared to various analogs.

FIG. 3 is a graphical representation showing a comparison of the stability of C8-Endo1 (1) and C8(Dmt)-Endo1 (7) with the stability of the parent peptides (Endo1 and (Dmt¹)Endo respectively), to degradation over 2 hours by an homogenate of 21 day old Caco-2 cells, was determined as a guide for assessing improved metabolic stability.

FIG. 4(a) is a graphical representation showing the apparent permeability of Caco-2 cells to endomorphin-1, a derivative of endomorphin and a derivative of an endomorphin analog. Caco-2 cells form polarised monolayers that mimic the brush border of the small intestine and are an accepted tool for assessing likely oral bioavailability. Caco-2 cells were used to assess the apparent permeability of the cells as a measure of their likelihood of being absorbed across the small intestine. It has been demonstrated that a good correlation exists between the Pₐₚₚ of a compound measured in Caco-2 cells and the intestinal absorption in humans and rats (Artursson and Karlsonn, Biochem. Biophys. Res. Comm. 1991, 175(3), 880-885). It was proposed that compounds that exhibit Pₐₚₚ values of <2×10⁻⁷ can be considered to have <1% absorption in humans. Compounds with Pₐₚₚ values of >2×10⁻⁶ can be considered to have 100% absorption in humans. These values are approximate but provide a good guide for the results of the assay. The Pₐₚₚ values of C8-Endo1 (1) and C8-(Dmt)-Endo1 (7) are displayed. While 1 showed a significantly improved apparent permeability compared to the parent peptide, the compound 7 showed a surprisingly lower Pₐₚₚ. The modification from Tyr to Dmt was not expected to greatly alter the lipophilicity of the compound and so the drop in permeability was not expected. The reason for the lower permeability of the DMT analogs is not known but it may be related to increased protein binding or efflux transporter activity.

FIG. 4(b) is a graphical representation showing the apparent permeability of Caco-2 cells to endomorphin, derivatives of endomorphin and a positive control, propranolol. In particular, in FIG. 4(b), ‘endo’ refers to Endo1 from Table 2, ‘c8-endo’ refers to compound 1 from Table 2, ‘lac-endo’ refers to compound 12 from Table 2, ‘lac-k-endo’ refers to compound 48 from Table 2, ‘lac-k-c12-endo’ refers to compound 49 from Table 2, ‘lac-K-lac-Endo’ refers to compound 50 from Table 2, ‘C12-K-lac-Endo’ refers to compound 51 from Table 2, ‘Lac-C12-Endo’ refers to compound 52 from Table 2.

FIG. 4(c) is a graphical representation showing the apparent permeability of Caco-2 cells to the lactose derivative of endomorphin, ‘lac-endo’ (compound 12 from Table 2) in the presence and absence of lactose, glucose and galctose.

FIG. 4(d) is a graphical representation showing the concentration of a lactose derivative of endomorphin, ‘lac-endo’ (compound 12 from Table 2), and endomorphin itself in the blood of male rats fed the compound via oral gavage. The graph shows that the concentration of endomorphin found in the blood was approximately 0 μM, whereas the highest recorded concentration for ‘lac-endo’ was found after 60 minutes.

FIG. 5(a)-(c) are graphical representations showing in vivo bio-distribution of 1 in rats. The in vivo biodistribution of 1 was completed with the intravenous of ³H-labelled 1 and Endo1 to male Sprague-Dawley rats. After 15, 30 and 60 minutes, 5 rats were sacrificed and the brain FIG. 5(a), liver FIG. 5(b) and blood FIG. 5(c) of each rat were removed following intravenous administration. The organs were subsequently homogenised in buffer and a sample removed and placed in tissue solubiliser for 3 days. After this time a small sample was removed and treated with bleach over night to remove any colour from the solution and the resulting sample dissolved in scintillation fluid and the radioactivity measured by liquid scintillation. The i.v. data clearly shows that 1 crosses the blood brain barrier more effectively than the parent peptide with >10% of the label appearing in the brain after 15 minutes compared to only 4% for the parent peptide. Compound 1 also appears to accumulate less in the liver than the parent peptide. The apparent increase in blood levels of the parent peptide after 60 minutes is likely due to the breakdown of the compound and release of the radiolabeled acetate into the blood stream.

FIG. 6(a)-(c) are graphical representations illustrating the biodistribution of Compound 1 after oral administration compared to that of Endo1. The in vivo biodistribution of 1 was completed with oral administration of ³H-labelled 1 and Endo1 to male Sprague-Dawley rats. After 15, 30 and 60 minutes, 5 rats were sacrificed and the brain, liver, blood, kidneys, stomach, spleen, small intestine and large intestine were removed following oral administration. The organs were subsequently homogenised in buffer and a sample removed and placed in tissue solubiliser for 3 days. After this time a small sample was removed and treated with bleach over night to remove any colour from the solution and the resulting sample dissolved in scintillation fluid and the radioactivity measured by liquid scintillation. The biodistribution of 1 following oral administration shows a 6-fold increase in serum levels compared to the native peptide over the time of the experiment. Metabolism in the liver of 1 again appeared less significant and there was evidence of delivery of the peptide to the brain.

FIG. 7 is a graphical representation illustrating the ability of native endomorphin-1 (Endo1) and analogs 7, 8 and 9 to displace ³H-DAMGO from the μ-opioid receptor.

FIG. 8 is a graphical representation showing plasma stability (in minutes) of derivatised endomorphin analogs 7, 8 and 9.

FIG. 9A is a graphical representation showing mean(±SEM) PWTs for the ipsilateral hindpaw of CCI-rats over 3 h following i.v. administration of vehicle (n=3), Compound 8 (0.3 mg/kg; n=4), Compound 8 (1 mg/kg; n=10), Compound 8 (3 mg/kg; n=3).

FIG. 9B is a graphical representation showing a comparison of the mean(±SEM) anti-allodynic response versus time curves produced by Endo1 (1 mg/kg; n=7) and Compound 8 (1 mg/kg; n=10) in the ipsilateral hindpaw of CCI-rats.

FIG. 10 is a graphical representation showing mean(±SEM) PWTs in the contralateral hindpaw of CCI-rats over 3 h following i.v. administration of vehicle (n=3), Endo1 (1 mg/kg; n=7), or Compound 8 (0.3 mg/kg; n=4), Compound 8 (1 mg/kg; n=10), Compound 8 (3 mg/kg; n=3).

FIG. 11 is a graphical representation showing mean(±SEM) PWTs for the ipsilateral hindpaw over 3 h following i.v. administration of single bolus doses of vehicle (n=3), Endo1 (1.6 μmol/kg; n=5), or Compound 8 (1.2 μmol/kg; n=8), in drug-naive CCI-rats.

FIG. 12 is a graphical representation showing mean(±SEM) area under the dose-normalised PWT versus time curve (AUC) for the ipsilateral hindpaw over 3 h following i.v. administration of vehicle (n=3), Endo1 (1.6 μmol/kg; n=5), or Compound 8 (1.2 μmol/kg; n=8), in drug-naive CCI-rats.

FIG. 13 is a graphical representation showing mean(±SEM) PWTs for the contralateral hindpaw over 3 h following i.v. administration of vehicle (n=3), Endo1 (1.6 μmol/kg; n=5), or Compound 8 (1.2 μmol/kg; n=8), in drug-naive CCI-rats.

FIG. 14(a) is a graphical representation showing mean(±SEM) PWTs for the ipsilateral hindpaw over 3 h following s.c. administration of vehicle or Compound 8 (0.1-10 mg/kg), in drug-naive CCI-rats.

FIG. 14(b) is a graphical representation showing mean(±SEM) PWTs for the contralateral hindpaw over 3 h following s.c. administration of vehicle or Compound 8 (0.3-3 mg/kg), in drug-naive CCI-rats.

FIG. 15 is a graphical representation showing the log dose vs. % inhibition of gastrointestinal motility response curve for single bolus i.v. doses of morphine and Compound 8. The average gradient for the curve corresponding to Compound 8 is lower than the average gradient for the curve corresponding to morphine.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

Sequences of amino acid residues described herein are understood to read conventionally, with the nominal ‘N-terminus’ shown on the left, and the nominal ‘C-terminus’ shown on the right.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 20%, or 10% to a reference quantity, level, value, dimension, size, or amount.

As used herein, the term “amino acid” refers to a molecule that comprises at least one primary or secondary amine and at least one acid.

The term “analgesia” is used herein to describe states of reduced pain perception, including absence from pain sensations as well as states of reduced or absent sensitivity to noxious stimuli. Such states of reduced or absent pain perception are induced by the administration of a pain-controlling agent or agents also called “analgesics” and occur without loss of consciousness, as is commonly understood in the art. The term analgesia encompasses the term “antinociception”, which is used in the art as a quantitative measure of analgesia or reduced pain sensitivity in animal models.

As used herein, the term “C₁₋₄alkyl” as used alone or as part of a group such as “di(C₁₋₄alkyl)amino” refers to straight chain, branched or cyclic alkyl groups having from 1 to 4 carbon atoms. Examples of such alkyl groups include methyl, ethyl, n-propyl, isopropyl, n-butyl, cyclopentyl and cyclohexyl. Similarly, C₁₋₆, C₁₋₈ and C₁₋₁₀ alkyl, for example, refer to groups having 1 to 6, 1 to 8, and 1 to 10 carbon atoms, respectively.

As used herein, the term “arylC₁₋₄alkyl” refers to groups formed from C₁₋₄ straight chain or branched alkyl groups substituted with an aromatic ring. Examples of C₁₋₄alkylaryl include phenylmethyl(benzyl), 2-phenylethyl(phenethyl), 3-phenylpropyl and 1-phenylprop-2-yl(phenylisopropyl).

Throughout this specification, unless the context requires otherwise, the words “comprise”, “comprises” and “comprising” will be understood to imply the inclusion of a stated step or element or group of steps or elements but not the exclusion of any other step or element or group of steps or elements.

As referred to herein, a “cyclitol” is any cycloalkane comprising one hydroxyl group on each of three or more ring atoms. Notable members are the inositols (1,2,3,4,5,6-cyclohexanehexyls) and their derivatives.

As used herein: ‘Gal’ refers to galactopyranosyl; ‘Glc’ refers to glucopyranosyl; and ‘Man’ refers to mannopyranosyl.

By “effective amount”, in the context of treating or preventing pain is meant the administration of that amount of active to an individual in need of such treatment or prophylaxis, either in a single dose or as part of a series, that is effective for the prevention of pain, holding pain in check, and/or treating existing pain. The effective amount will vary depending upon the health and physical condition of the individual to be treated, the taxonomic group of individual to be treated, the formulation of the composition, the assessment of the medical situation, and other relevant factors. An “effective amount” of an active may be the amount administered to a subject in need thereof that is sufficient to reduce pain and/or reduce a response to pain (for example tactile allodynia). It is expected that the amount will fall in a relatively broad range that can be determined through routine trials. In some instances, administration of an “effective amount” of active may produce a 50% reduction in pain.

As used herein, the term “heterocyclic amino acid” refers to a heterocyclic group that comprises, or has appended, at least one primary or secondary amine and at least one acid.

The term “heterocyclic group” as used herein refers to mono or bicyclic rings or ring systems which include at least one hetero atom selected from N, S and O. The rings or ring systems generally include 1 to 9 carbon atoms in addition to the heteroatom(s) and may be saturated, unsaturated, aromatic or pseudoaromatic.

Non-limiting examples of 5-membered monocyclic heterocycles include pyrrolines, pyrrolidines, pyrroles, imidazoles, oxazoles, triazoles, tetrazoles, thiazoles, isoxazoles, isothiazolyl, pyrazolyl, oxadiazoles, thiadiazoles and examples of 6-membered monocyclic heterocycles include pyridines, pyrimidines, pyridazines, pyrazines and triazines, piperidines, piperazines, morpholines, each of which may be optionally substituted with C₁₋₆alkyl, C₁₋₆alkoxy, C₃₋₆alkenyl, C₃₋₆alkynyl, halo, hydroxy, mercapto, trifluoromethyl, amino, cyano, C₁₋₆alkylamino or di(C₁₋₆alkyl)amino. Examples of 9- and 10-membered nitrogen heterocycles include indoles, benzoxazoles, benzothiazoles, benzisoxazoles, benzisothiazoles, indazoles, benzimidazoles, purines, pteridines, indolizines, isoquinolines, quinolines, quinoxalines, cinnolines, phthalazines, quinazolines, benzotriazines and the like, each of which may be optionally substituted with C₁₋₆alkyl, C₁₋₆alkoxy, C₃₋₆alkenyl, C₃₋₆alkynyl, halo, hydroxy, mercapto, trifluoromethyl, amino, cyano, C₁₋₆alkylamino or di(C₁₋₆alkyl)amino. Preferred heterocyclic rings include (optionally substituted) pyrrolidines, isoxazoles, isothiazoles, 1,3,4-oxadiazoles, 1,3,4-thiadiazoles, 1,2,4-oxadiazoles, 1,2,4-thiadiazoles, oxazoles, thiazoles, pyridines, pyridazines, pyrimidines, pyrazines, 1,2,4-triazines, 1,3,5-triazines, benzoxazoles, benzothiazoles, benzisoxazoles, benzisothiazoles, quinolines and quinoxalines.

As used herein, the terms “lipid” and variants such as “lipo” and “lipidic” refer to members of a large and diverse group of oils, fats and fat like substances that occur in living organisms and that characteristically are soluble in lipid solvents (any non-polar solvent that can be used to extract lipids from tissues or other materials) but are only sparingly soluble in aqueous solvents. Lipidic groups may be optionally substituted, branched or linear, and saturated or unsaturated. Lipids may also include one or more heterocyclic, cycloalkyl, or aromatic ring systems, including fused ring systems such as, for example, steroids. Examples of lipidic groups encompassed within the scope of the present invention are C₄₋₂₂alkyl, C₄₋₂₂alkenyl or C₄₋₂₂alkynyl groups. More specific examples of lipidic groups include: hexyl, heptyl, octyl, nonyl, decyl, undecanyl, dodecanyl, tetradecanyl, tetradecenyl, tetradecadienyl, hydroxy-tetradecenyl, methyl-tetradecenyl, hexadecenyl, hexadecadienyl, hexadecatrienyl, methyl-hexadecanyl, methyl-hexadecenyl, octadecanyl, hydroxy-octadecanyl, di-hydroxy-octadecanyl, octadecenyl, octadecadienyl, octadecatrienyl, octadecatetraenyl, eicosanyl, eicosaenyl, eicosadienyl, eicosatrienyl, eicosatetraenyl, hydroxy-eicosaenyl, docosanyl, docosenyl, docosadienyl, hydroxy-docosenyl, tetracosanyl, tetracosenyl, hexacosanyl, hexacosenyl and the like.

As used herein, the term “lipo-amino acid” refers to an amino acid that has a lipidic side chain. The lipo-amino acid may be an L-amino acid, D-amino acid, or a mixture of L- and D-amino acids including racemic mixtures.

“Nociceptive pain” refers to the normal, acute pain sensation evoked by activation of nociceptors located in non-damaged skin, viscera and other organs in the absence of sensitization.

The term “opioid receptor agonist” as used herein refers to any compound which upon administration is capable of binding to an opioid receptor and causing agonism, partial agonism or mixed agonism/antagonism of the receptor. Metabolites of administered compounds are also encompassed by the term opioid receptor agonists. Preferred opioid receptor agonists are those that agonize opioid receptors to produce analgesia. Opioid analgesics include opiate alkaloids which may be isolated from opium and synthetic derivatives or analogs thereof.

The term “pain” as used herein is given its broadest sense and includes an unpleasant sensory and emotional experience associated with actual or potential tissue damage, or described in terms of such damage and includes the more or less localized sensation of discomfort, distress, or agony, resulting from the stimulation of specialized nerve endings. There are many types of pain, including, but not limited to, lightning pains, phantom pains, shooting pains, acute pain, inflammatory pain, neuropathic pain, nociceptive pain, complex regional pain, neuralgia, neuropathy, and the like (Dorland's Illustrated Medical Dictionary, 28^{th} Edition, W. B. Saunders Company, Philadelphia, Pa.). The goal of treatment of pain is to reduce the degree of severity of pain perceived by a treatment subject.

By “pharmaceutically acceptable carrier” is meant a solid or liquid filler, diluent or encapsulating substance that may be safely used in topical, local or systemic administration.

The term “pro-drug” is used in its broadest sense and encompasses those compounds that are converted in vivo to an opioid receptor agonist according to the invention. Such compounds would readily occur to those of skill in the art, and include, for example, compounds where a free hydroxy group is converted into an ester derivative. Pro-drug forms of compounds may be utilised, for example, to improve bioavailability, mask unpleasant characteristics such as bitter taste, alter solubility for intravenous use, or to provide site-specific delivery of the compound.

As used herein the term “pro-drug linkage” refers to a chemical linkage that is capable of being cleaved by various mechanisms, including metabolic processes. Examples of pro-drug linkages include ester, imino and carbamate linkages.

The term “pharmaceutically compatible salt” as used herein refers to a salt which is toxicologically safe for human and animal administration. The salt may be an acid addition salt, or the salt formed following reaction with a base such as sodium hydrogencarbonate. This salt may be selected from a group including, for example, hydrochlorides, hydrobromides, hydroiodides, sulphates, bisulphates, nitrates, citrates, tartrates, bitartrates, phosphates, malates, maleates, napsylates, fumarates, succinates, acetates, terephthalates, pamoates and pectinates.

As used herein, the term “saccharide” refers to the series of compounds comprising carbon, hydrogen, and oxygen in which the atoms of the latter two elements are in the approximate ratio of 2:1, especially those containing the group C₆H₁₀O₅, for example, monosaccharides and disaccharides. Examples of saccharide substituents that fall within the scope of the present invention are: lactosyl, glucopyranosyl, mannopyranosyl, galactopyranosyl, 2-deoxy-2-acetamido-glucopyranosyl, 2-deoxy-2-acetamido-galactopyranosyl, maltosyl, glucoronyl and galacturonyl. The term “monosaccharide”, as used herein, refers to polyhydroxy aldehydes. H—[CHOH]_{U}—CHO or polyhydroxy ketones H—[CHOH]_{U}—CO—[CHOH]_{V}—H with three or more carbon atoms, wherein U and V are each independently positive, non-zero integers. The generic term ‘monosaccharide’ includes aldoses, dialdoses, aldoketoses, ketoses and diketoses, as well as deoxy sugars and amino sugars, and their derivatives, provided that the parent compound has a carbonyl group or potential carbonyl group. Monosaccharides with an aldehydic carbonyl or potential aldehydic carbonyl group are called aldoses; those with a ketonic carbonyl or potential ketonic carbonyl group, ketoses. The term ‘potential aldehydic carbonyl group’ refers to the hemiacetal group arising from ring closure. Likewise, the term ‘potential ketonic carbonyl group’ refers to the hemiketal structure. Cyclic hemiacetals or hemiketals of sugars with a five-membered (tetrahydrofuran) ring are called furanoses, those with a six-membered (tetrahydropyran) ring pyranoses. Monosaccharides containing two (potential) aldehydic carbonyl groups are called dialdoses. Monosaccharides containing two (potential) ketonic carbonyl groups are termed diketoses. Monosaccharides containing a (potential) aldehydic group and a (potential) ketonic group are called ketoaldoses. Monosaccharides in which an alcoholic hydroxy group has been replaced by a hydrogen atom are called deoxy sugars. Monosaccharides in which an alcoholic hydroxy group has been replaced by an amino group are called amino sugars. When the hemiacetal hydroxy group is replaced, the compounds are called glycosylamines. Another example of an aminated saccharide is glucosamine, wherein a secondary alcohol functionality in glucose has been substituted by an amine functionality. The polyhydric alcohols arising formally from the replacement of a carbonyl group in a monosaccharide with a CHOH group are termed alditols. Monocarboxylic acids formally derived from aldoses by replacement of the aldehydic group by a carboxy group are termed aldonic acids. Oxo carboxylic acids formally derived from aldonic acids by replacement of a secondary CHOH group by a carbonyl group are called ketoaldonic acids. Monocarboxylic acids formally derived from aldoses by replacement of the CH₂OH group with a carboxy group are termed uronic acids. The structures of such monocarboxylic acids may be further elaborated by, for example, transformation of the carboxylic acid functionality into an amide functionality. An example of a molecule resulting from such a transformation is glucuronamide. The dicarboxylic acids formed from aldoses by replacement of both terminal groups (CHO and CH₂OH) by carboxy groups are called aldaric acids. The monosaccharides may be in D or L form. Particular examples of monosaccharides are provided as follows: an example of an aldotriose is glyceraldehyde; examples of aldotetraoses are erythrose and threose; examples of pentoses are ribose, arabinose, xylose and lyxose, examples of hexoses are allose, altrose, glucose, mannose, gulose, idose, galactose and talose, examples of aminosugars are N-acetyl-glucosamine, N-acetyl-galactosamine, and N-acetyl-mannosamine; an example of a deoxy sugar is fucose, an example of a ketopentose is ribulose, and example of a ketohexose is fructose, examples of uronic acids are galacturonic acid, mannuronic ancd, glucuronic acid and iduronic acid, other carboxylic acid containing monosaccharides are sialic acid and KDO. Other types of derivatives of monosaccharides include alditols, for example: xylitol, sorbitol, mannitol, galactitol, and glucitol etc. The term “disaccharide”, as used herein, refers to a saccharide made up of two monosaccharides or simple sugars. Non-limiting examples of disaccharides include melibiose (Galα(1→6′)Glc), isomaltose (Glcα(1→6′)Glc), maltose (Glcα(1→4)Glc), mannobiose (Manβ(1→4′)Man), gentibiose (Galβ(1→6′)Glc), lactose (Galβ(1→4′)Glc), allolactose (Galβ(1→6′)Glc), cellobiose (Glcβ(1→4′)Glc), sucrose and trehalose (Glcα(1→1′)Glc).

As used herein “side chain” refers to a part of a molecule attached to a core structure which may vary for a given core, for example, the peptide or protein side chains are variable parts of amino acids extending from the peptide backbone.

The term “subject” or “individual” or “patient”, used interchangeably herein, refer to any subject, preferably a vertebrate subject, and even more preferably a mammalian subject, for whom therapy or prophylaxis is desired. Suitable vertebrate animals that fall within the scope of the invention include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment or prophylaxis for pain, especially moderate or severe pain. However, it will be understood that the aforementioned terms do not imply that symptoms are present.

The term “substituted” and variations such as “optionally substituted” as used herein, unless otherwise indicated, means that a group may include one or more substituents. Illustrative substituents of this type include halo, C₁₋₂₂alkyl, C₂₋₂₂alkenyl, C₂₋₂₂alkynyl, C₁₋₂₂alkoxy, haloC₁₋₄alkyl, hydroxyC₁₋₄alkyl, C₁₋₄alkoxy, C₁₋₂₂acyl, C₁₋₂₂acyloxy, hydroxy, aryl, amino, amido, azido, nitro, nitroso, cyano, carbamoyl, trifluoromethyl, mercapto, aryloxy, formyl, carbamoyl, C₁₋₂₂alkylsulphonyl, C₁₋₆arylsulphonyl, C₁₋₂₂alkylsulphonamido, C₁₋₆arylsulphonamido, C₁₋₂₂alkylamino, di(C₁₋₂₂alkyl)amino, C₁₋₄alkoxycarbonyl, arylC₁₋₄alkyloxy, C₄₋₂₂alkyloxycarbonyl, C₄₋₂₂acylthio, C₄₋₂₂acylamino, C₁₋₆alkylsulfinyl, C₄₋₂₂alkylthio. Another example of a substituent that may be optionally substituted on a group is an oxo moiety

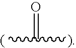

The oxo moiety is a divalent substituent occupying two positions of substitution on the group that is optionally substituted.

As used herein, the term “aryl” refers to optionally substituted monocyclic, bicyclic, and biaryl carbocyclic aromatic groups, of 6 to 14 carbon atoms, covalently attached at any ring position capable of forming a stable covalent bond, certain preferred points of attachment being apparent to those skilled in the art. Examples of monocyclic aromatic groups include phenyl, toluoyl, xylyl and the like, each of which may be optionally substituted with C₁₋₆acyl, C₁₋₆alkyl, C₁₋₆alkoxy, C₂₋₆alkenyl, C₂₋₆alkynyl, C₁₋₆alkylsulphonyl, arylsulphonyl, C₁₋₆alkylsulphonamido, arylsulphonamido, halo, hydroxy, mercapto, trifluoromethyl, carbamoyl, amino, azido, nitro, cyano, C₁₋₆alkylamino or di(C₁₋₆alkyl)amino. Examples of bicyclic aromatic groups include 1-naphthyl, 2-naphthyl, indenyl and the like, each of which may be optionally substituted with C₁₋₆acyl, C₁₋₆alkyl, C₁₋₆alkoxy, C₂₋₆alkenyl, C₂₋₆alkynyl, C₁₋₆alkylsulphonyl, arylsulphonyl, C₁₋₆alkylsulphonamido, arylsulphonamido, halo, hydroxy, mercapto, trifluoromethyl, carbamoyl, amino, azido, nitro, cyano, C₁₋₆alkylamino or di(C₁₋₆alkyl)amino. Examples of biaryl aromatic groups include biphenyl, fluorenyl and the like, each of which may be optionally substituted with C₁₋₆acyl, C₁₋₆alkyl, C₁₋₆alkoxy, C₂₋₆alkenyl, C₂₋₆alkynyl, C₁₋₆alkylsulphonyl, arylsulphonyl, C₁₋₆alkylsulphonamido, arylsulphonamido, halo, hydroxy, mercapto, trifluoromethyl, carbamoyl, amino, azido, nitro, cyano, C₁₋₆alkylamino or di(C₁₋₆alkyl)amino.

As used herein, the term “heteroaryl” refers to optionally substituted monocyclic, bicyclic and biaryl carbocyclic aromatic groups wherein one or more of the carbon atoms forming the carbocycle have been substituted by an atom selected from the group consisting of: O, N and S. Examples of heteroaryl groups are 2-thiophenyl, 5-quinolinyl, 3-imidazolyl and 2-pyrimidinyl.

As used herein, the terms “C₄₋₂₂alkoxy” and “C₄₋₂₂alkyloxy” refer to straight chain or branched alkoxy groups having from 4 to 22 carbon atoms. Examples of C₄₋₂₂alkoxy include hexyloxy, octyloxy, decyl, cyclohexyloxy, and the different butoxy isomers. Similarly, C₁₋₄, C₁₋₈ and C₁₋₁₀ alkoxy refer to groups having 1 to 4, 1 to 8, and 1 to 10 carbon atoms, respectively.

As used herein, the term “aryloxy” refers to an “aryl” group attached through an oxygen bridge. Examples of aryloxy substituents include phenoxy, biphenyloxy, naphthyloxy and the like.

As used herein, the term “arylC₁₋₄alkyloxy” refers to an “arylC₁₋₄alkyl” group attached through an oxygen bridge. Examples of “arylC₁₋₄alkyloxy” groups are benzyloxy, phenethyloxy, naphthylmethyleneoxy, biphenylmethyleneoxy and the like.

As used herein, the terms “C₄₋₂₂acyl” refers to straight chain or branched, aromatic or aliphatic, saturated or unsaturated acyl groups having from 1 to 22 carbon atoms. Examples of C₄₋₂₂acyl include butanoyl, sec-butanoyl, pentanoyl, pivaloyl, hexanoyl, heptanoyl, octanoyl, nonanoyl, decanoyl, undecanoyl, dodecanoyl, benzoyl and 2-phenylacetyl. Similarly, C₁₋₄, C₁₋₆ and C₁₋₈ acyl refer to groups having 1 to 4, 1 to 6, and 1 to 8 carbon atoms, respectively.

As used herein, the term “C₄₋₂₂alkyloxycarbonyl” refers to a “C₄₋₂₂alkyloxy” group attached through a carbonyl group. Examples of “C₄₋₂₂alkyloxycarbonyl” groups include butylformate, sec-butylformate, hexylformate, octylformate, decylformate, cyclopentylformate and the like.

As used herein, the term “C₄₋₂₂alkenyl” refers to groups formed from C₄₋₂₂ straight chain, branched or cyclic alkenes. Examples of C₄₋₂₂alkenyl include butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1,3-butadienyl, 1-4, pentadienyl, 1,3-cyclopentadienyl, 1,3-hexadienyl, 1,4-hexadienyl, octenyl isomers, decenyl isomers, undecenyl isomers, dodecenyl isomers, 1,3-cyclohexadienyl and 1,4-cyclohexadienyl. Similarly, C₂₋₄, C₂₋₆ and C₂₋₁₀ alkenyl, for example, refer to groups having 2 to 4, 2 to 6, and 2 to 10 carbon atoms, respectively.

As used herein, the term “C₄₋₂₂alkynyl” refers to groups formed from C₄₋₂₂ straight chain or branched groups as previously defined which contain a triple bond. Examples of C₄₋₂₂alkynyl include 3-hexynyl, 2-hexynyl, 2- or 3-butynyl, octynyl and decynyl. Similarly, C₂₋₄, C₂₋₆ and C₂₋₁₀ alkynyl, for example, refer to groups having 2 to 4, 2 to 6, and 2 to 10 carbon atoms, respectively.

As used herein, the term “arylC₁₋₄alkyl” refers to groups formed from C₁₋₄ straight chain, branched alkanes substituted with an aromatic ring. Examples of arylC₁₋₄alkyl include phenylmethyl(benzyl), ethylphenyl, propylphenyl and isopropylphenyl.

As used herein, the term “C₄₋₂₂alkylthio” refers to straight chain or branched alkyl groups having from 4 to 22 carbon atoms attached through a sulfur bridge. Examples of C₄₋₂₂alkylthio include hexylthio, octylthio, decylthio, sec-butylthio, cyclohexylthio, different butylthio isomers and the like. Similarly, C₁₋₄, C₁₋₆ and C₁₋₈ alkylthio refer to groups having 1 to 4, 1 to 6, and 1 to 8 carbon atoms, respectively.

As used herein, the term “C₄₋₂₂alkylsulfinyl” refers to a “C₄₋₂₂alkyl” group attached through a sulphinyl bridge. Examples of “C₄₋₂₂alkylsulfinyl” groups include hexylsulphinyl, octylsulphinyl, decylsulphinyl, sec-butylsulphinyl, nonylsulphinyl and the like.

As used herein, the term “C₄₋₂₂alkylsulfonyl” refers to a “C₄₋₂₂alkyl” group attached through a sulphonyl bridge. Examples of “C₄₋₂₂alkylsulfonyl” groups include octylsulphonyl, hexylsulphonyl, decylsulphonyl and the like.

As used herein, the term “C₄₋₂₂alkylsulphonamido” refers to a “C₄₋₂₂alkylsulphonyl” group wherein the “C₄₋₂₂alkylsulphonyl” group is in turn attached through a nitrogen atom. Examples of “C₄₋₂₂alkylsulphonamido” groups include hexylsulphonamido, octylsulphonamido, decylsulphonamido and the like.

As used herein, the term “C₄₋₂₂alkylamino” refers to a “C₄₋₂₂alkyl” group attached through an amine bridge. Examples of “C₄₋₂₂alkylamino” include hexylamino, heptylamino, octylamino, nonylamino, decylamino, undecylamino, dodecylamino and the like.

As used herein, the term “di(C₄₋₂₂alkyl)amino” refers to two, independently selected, “C₄₋₂₂alkyl” groups having the indicated number of carbon atoms attached through an amine bridge. Examples of “di(C₄₋₂₂alkyl)amino” include diethylamino, dihexylamino, dioctylamino, N-hexyl-N-octyl-amino, N-propyl-N-hexylamino, N-cyclopentyl-N-octylamino and the like.

As used herein, the term “C₄₋₂₂acylamino” refers to a “C₄₋₂₂acyl” group wherein the “C₄₋₂₂acyl” group is in turn attached through a nitrogen atom. The nitrogen atom may itself be substituted with a “C₁₋₆alkyl” or “aryl” group. Examples of “C₄₋₂₂acylamino” include hexylcarbonylamino(heptamido), cyclopentylcarbonyl-amino(methyl), benzamido, propylcarbonylamino, biphenylcarbonylamino (eg 4-phenylbenzamido), naphthylcarbonylamino and the like.

As used herein, the term “C₄₋₂₂acyloxy” refers to a “C₄₋₂₂acyl” group wherein the “C₄₋₂₂acyl” group is in turn attached through an oxygen atom. Examples of “C₄₋₂₂acyloxy” include hexylcarbonyloxy(heptanoyloxy), cyclopentylcarbonyloxy, benzoyloxy, 4-chlorobenzoyloxy, decylcarbonyloxy(undecanoyloxy), propylcarbonyloxy(butanoyloxy), octylcarbonyloxy(nonanoyloxy), biphenylcarbonyloxy (eg 4-phenylbenzoyloxy), naphthylcarbonyloxy (eg 1-naphthoyloxy) and the like.

As used herein, the term “C₄₋₂₂acylthio” refers to a “C₄₋₂₂acyl” group wherein the “C₄₋₂₂acyl” group is in turn attached through a sulphur atom. Examples of “C₄₋₂₂acylthio” include hexylcarbonylthio(heptanoylthio), cyclopentylcarbonylthio, benzoylthio, 4-chlorobenzoylthio, acetylthio, propylcarbonylthio(butanoylthio), 2-chloroacetylthio, biphenylcarbonylthio (eg 4-phenylbenzoylthio), naphthylcarbonylthio (eg 1-naphthoylthio) and the like.

As used herein, the term “carboxyC₁₋₆alkyl” refers to a “C₁₋₆alkyl” group substituted with at least one carboxy moiety at any position within that group. An example of a “carboxyC₁₋₆alkyl” group is the “carboxymethyl” group:

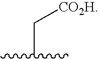

As used herein, the term “aminoC₁₋₆alkyl” refers to a “C₁₋₆alkyl” group substituted with at least one amino moiety at any position within that group. An example of an “aminoC₁₋₆alkyl” group is the “aminomethyl” group.

As used herein, the term ‘endomorphin analog’ refers to an oligopeptide that resembles the oligopeptide of an endomorphin, such as endomorphin-1 or endomorphin-2. Typically, the derivatised endomorphin analogs of the present invention exhibit the properties of similar size and/or similar number and nature of amino acid residues relative to an endomorphin. Preferably, the derivatised endomorphin analogs of the present invention are no more than five times the molecular weight of either endomorphin-1 or endomporphin-2. More preferably, the derivatised endomorphin analogs of the present invention are no more than 3 times the molecular weight of either endomorphin-1 or endomorphin-2. Moreover, derivatised endomorphin analogs of the present invention typically have a relationship with an opioid receptor that is similar to the relationship of either endomorphin-1 or endomorphin-2 with that opioid receptor. An example of such a relationship is antagonism of the μ-opioid receptor by endomorphin-1.

As used herein, the term ‘linker moiety’ refers to an optionally substituted divalent group capable of linking two other moieties. An example of a ‘linker moiety’ is an alkylene group. A particular example of a linker moiety is the divalent 1,4-butylene group. Another example of a linker group is optionally substituted C₁₋₄alkyleneacyl. A specific example of a C₁₋₄alkyleneacyl is represented by the following structure:

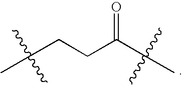

Another example of a linker group is optionally substituted C₁₋₄alkylenearyl. A specific example of a C₁₋₄alkylenearyl is represented by the following structure:

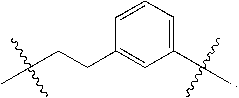

### 2. Compounds of the Present Invention

The present invention arises in part from the determination that derivatisation of an endomorphin, or of an endomorphin analog, with at least one lipid moiety and/or at least one saccharide moiety, significantly improves the cell permeability and/or stability of the endomorphin or endomorphin analog without abrogating its opioid receptor binding capacity. Accordingly, the present invention provides, in one aspect, derivatives of an endomorphin, or of an endomorphin analog, which comprise at least one moiety selected from a lipid, a cyclitol and a saccharide moiety.

The endomorphin analogs of the present invention may contain any conventional (for example, naturally occurring amino acids) or non-conventional amino acids (for examples of non-conventional amino acids see Table 1), including the D-form of the amino acids, amino acid derivatives and amino acid analogs, so long as the desired function and activity of the peptide or peptide analog is maintained. The choice of including an L- or a D-amino acid in the peptide or peptide analog depends, in part, on the desired characteristics of the peptide. For example, the incorporation of one or more D-amino acids can confer increased stability on a peptide and can allow a peptide to remain active in the body for an extended period of time. The incorporation of one or more D-amino acids can also increase or decrease the pharmacological activity of a peptide. Mixtures of L- and D-amino acids may also be used in the synthesis of a derivative of endomorphin, or of an endomorphin analog. An example of such a mixture is a racemate. The peptides and peptidomimetics may also be cyclised, since cyclisation may provide the peptide with superior properties over their linear counterparts. Furthermore, α-amino acids may be substituted with β-amino acids to similarly confer modulated pharmacological activity or stability on the peptide.

TABLE 1
Non-conventional amino acids:
Non-Conventional Amino Acids

| | |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

In one aspect, the invention provides derivatives of endomorphin, and of endomorphin analogs, comprising a moiety represented by formula I:

Q¹-P¹-Q³-Q⁴ formula I
wherein:
   Q¹ is selected from an optionally substituted phenolic amino acid residue;
   P¹ is an amino acid residue or is a linker moiety which is further substituted with a cyclitol, saccharide moiety and/or a lipidic group;
   Q³ is selected from an optionally substituted aromatic amino acid residue; and
   Q⁴ is selected from an optionally substituted aromatic amino acid residue;
with the proviso that at least one lipidic, cyclitol or saccharide moiety is conjugated to the compound comprising the moiety represented by formula I.

In a preferred embodiment, P¹ is an alpha- or beta-aliphatic or aromatic amino acid residue. In a further preferred embodiment, P¹ is an optionally substituted heterocyclic amino acid residue. In an especially preferred embodiment, P¹ is an optionally substituted alpha- or beta-proline residue. In another preferred embodiment, P¹ is an amino acid residue which is further substituted with a lipidic, cyclitol or saccharide moiety.

Preferably, the compounds provided by the invention may be represented by formula II:

L¹-Q¹-P¹-Q³-Q⁴-L²-A¹ formula II
wherein L¹, L² and A¹ may each be independently present or absent;
and wherein:
   Q¹ is selected from an optionally substituted phenolic amino acid residue;
   P¹ is an amino acid residue or is a linker moiety which is further substituted with a cyclitol, saccharide moiety and/or a lipidic group;
   Q³ is selected from an optionally substituted aromatic amino acid residue;
   Q⁴ is selected from an optionally substituted aromatic amino acid residue;
   A¹ is selected from an amine, amide and amide mimetic; and
   L¹ and L² are moieties represented by formula III:

Y¹—Y²—Y³—Y⁴ formula III
wherein:
   the, or each of, Y¹, Y², Y³ and Y⁴ is independently absent or present and is independently selected from:
   an amino acid moiety which is further substituted with a lipidic, cyclitol or saccharide moiety; and
   a linker moiety which may be further substituted with a lipidic, cyclitol or saccharide moiety;
and wherein at least one of P¹, Y¹, Y², Y³ or Y⁴ is an amino acid or linker moiety which is further substituted with a lipidic, cyclitol or saccharide moiety.

In a preferred embodiment, the side chains of the amino acid residues represented by Q³ and Q⁴ are independently selected from an optionally substituted arylC₁₋₄alkyl group and an optionally substituted 3-indolylmethyl group. The 3-indolylmethyl group may be represented by the following structure:

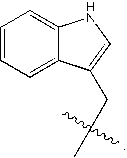

In an especially preferred embodiment, Q³ represents a 3-indolylmethyl group.

In a further preferred embodiment, the, or each, ‘linker’ moiety is represented by formula IV:

—W-Alk¹-T-Alk²- formula IV
wherein each of W, Alk¹, T and Alk² may be present or absent, provided that at least one of W, Alk¹, T or Alk² is present and wherein:
   W is selected from —N(R^{G})—, —NH(CO)—, —C(O)NH—, —S— and —O—, wherein R^{G} is hydrogen, optionally substituted C₁₋₆alkyl, optionally substituted arylC₁₋₄alkyl, optionally substituted aryl or optionally substituted heteroaryl;
   Alk¹ is selected from an optionally substituted C₁₋₄alkylene, optionally substituted C₂₋₅alkenylene, optionally substituted C₂₋₅alkynylene, optionally substituted arylene, optionally substituted heteroarylene and an optionally substituted C₁₋₄alkylenearyl, with the proviso that both W and T are not simultaneously present when Alk¹ is absent;
   T is selected from —NH—, —O—, —S—, —NHC(O)—, —C(O)NH—, —NHSO₂—, —C(R^{G})═N—NH—, —NHC(O)NH—, —NHC(S)NH—, —C(R^{G})═N— and —N═C(R^{G})—; and
   Alk² is selected from an optionally substituted C₁₋₄alkylene, optionally substituted C₂₋₅alkenylene, optionally substituted C₂₋₅alkynylene, optionally substituted arylene, optionally substituted heteroarylene and an optionally substituted C₁₋₄alkylenearyl.

In some embodiments, the lipidic group is a straight chain or branched, substituted or unsubstituted, alkyl, alkenyl or alkynyl group having from 4 to 22 carbon atoms. Such substituents may be optionally substituted, for example with one or more hydroxy, alkyl, alkoxy or halo groups. Examples of lipidic groups include: butyl, pentyl, hexyl, heptyl, octyl, nonyl, decyl, undecanyl, dodecanyl, tetradecanyl, tetradecenyl, tetradecadienyl, hydroxy-tetradecenyl, methyl-tetradeceny, hexadecenyl, hexadecadienyl, hexadecatrienyl, methyl-hexadecanyl, methyl-hexadecenyl, octadecanyl, hydroxy-octadecanyl, di-hydroxy-octadecanyl, octadecenyl, octadecadienyl, octadecatrienyl, octadecatetraenyl, eicosanyl, eicosaenyl, eicosadienyl, eicosatrieyl, eicosatetraenyl, hydroxy-eicosaenyl, docosanyl, docosenyl, docosadienyl, hydroxy-docosenyl, tetracosanyl, tetracosenyl, hexacosanyl and hexacosenyl.

Suitably, the cyclitol or saccharide, is selected from a general structure as provided below:

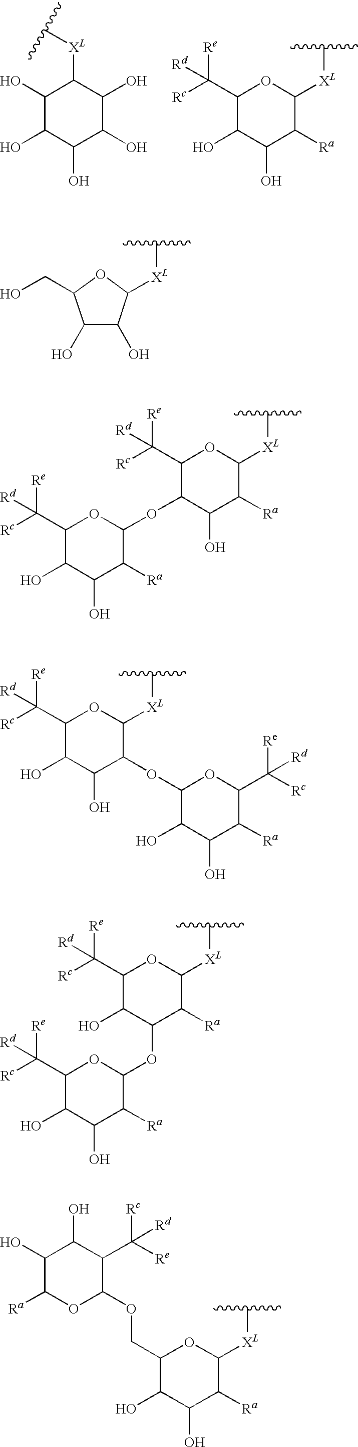

wherein:
   R^{c} and R^{d} are both hydrogen or combine to from a carbonyl function;
   R^{e} is an hydroxyl group, an amino group or a hydrogen atom;
   X^{L} is a linker moiety of formula IV; and
   R^{a} is selected from a hydroxyl group and an acetamide.

In some embodiments, the group P¹ is selected from the following moieties:

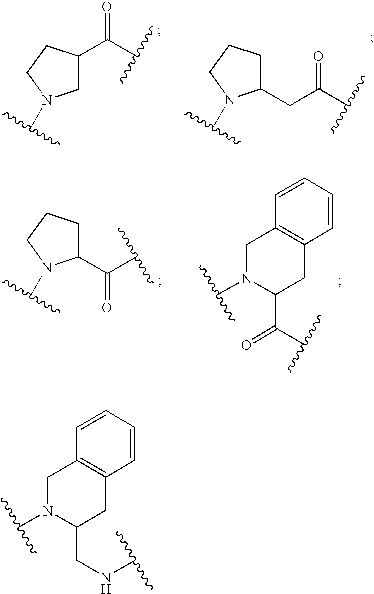

In other embodiments, the group P¹ is selected from the following moieties:

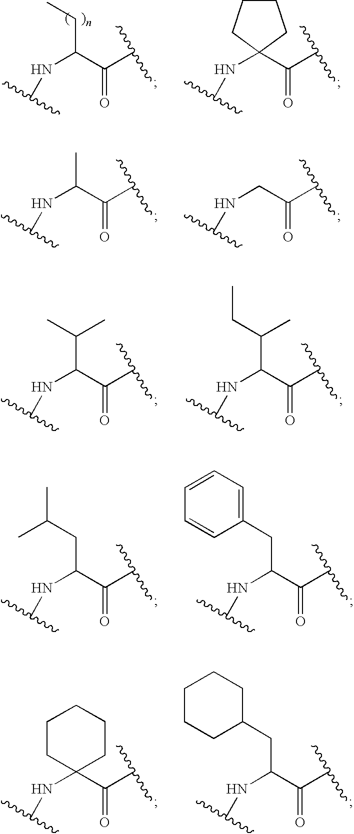

wherein n is an integer selected from 1 to 22.

In some embodiments, the derivatives of endomorphin, or of endomorphin analogs, are represented by formula V:

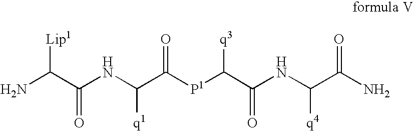

wherein:
   Lip¹ is a lipidic group;
   q¹ is a phenolic side chain;
   q³ and q⁴ are aromatic side chains; and
   P¹ is as defined above.

In some embodiments, the derivatives of endomorphin, or of endomorphin analogs, are represented by formula VI:

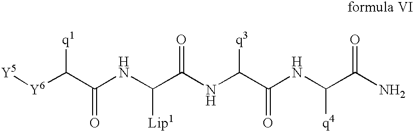

wherein:
   Lip¹, q¹, q³ and q⁴ are as defined above;
   Y⁵ is an amino acid moiety or a linking group further substituted with a cyclitol or saccharide group; and
   Y⁶ is a linker moiety.

In some embodiments, the derivatives of endomorphin, or of endomorphin analogs, are represented by formula VII:

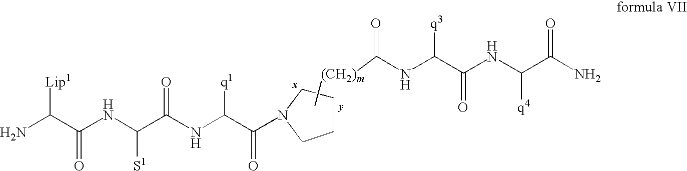

wherein:
   Lip¹, q¹, q³ and q⁴ are as defined above;
   S¹ is a cyclitol or saccharide group;
   m is an integer from 0 to 2; and
   x and y refer to points of substitution on the pyrrolidine ring.

In some embodiments, the derivatives of endomorphin, or of endomorphin analogs, are represented by formula VIII:

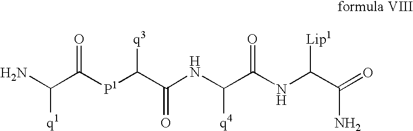

wherein:
   q¹, q³, q⁴, P¹ and Lip¹ are defined as above.

In some embodiments, the derivatives of endomorphin, or of endomorphin analogs, are represented by formula IX:

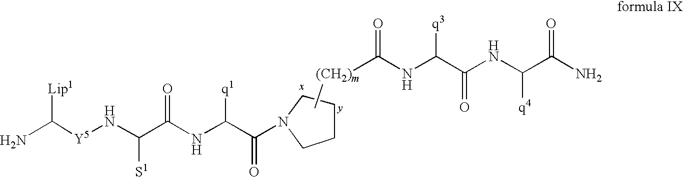

wherein:
   Lip¹, S¹, q¹, q³, q⁴, x, y and n are defined as above;
   m is an integer from 0 to 2; and
   Y⁵ is a linker moiety.

In some embodiments, the derivatives of endomorphin, or of endomorphin analogs, are represented by formula X:

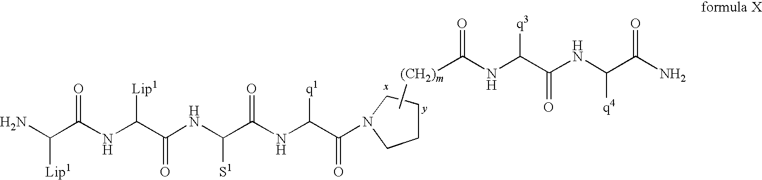

wherein:
   Lip¹ may be the same or different and are defined as above;
   m is an integer from 0 to 2; and
   S¹, q¹, q³, q⁴, x and y are defined as above.

In some embodiments, the derivatives of endomorphin, or of endomorphin analogs, are represented by formula XI:

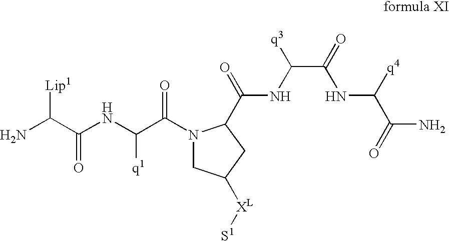

wherein:
   Lip¹, S¹, X^{L}, q¹, q³ and q⁴ are defined as above; and

In some embodiments, the derivatives of endomorphin, or of endomorphin analogs are represented by formula XII:

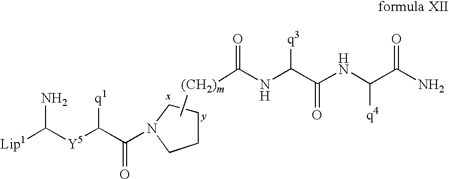

wherein
   Lip¹, q¹, q³, q⁴, x and y are as defined above;
   m is an integer from 0 to 2; and
   Y⁵ is a linking moiety.

In some embodiments, the derivatives of endomorphin, or of endomorphin analogs are represented by formula XIII:

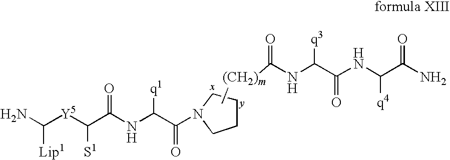

wherein:
   Lip¹, S¹, q¹, q³, q⁴, x and y are as defined above;
   m is an integer from 0 to 2; and
   Y⁵ is a linking moiety.

In some embodiments, the derivatives of endomorphin, or of endomorphin analogs are represented by formula XIV:

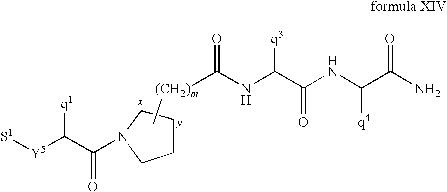

wherein:
   S¹, q¹, q³, q⁴, x and y are as defined above;
   m is an integer from 0 to 2; and
   Y⁵ is a linking moiety.

In some embodiments, the derivatives of endomorphin, or of endomorphin analogs are represented by formula XV:

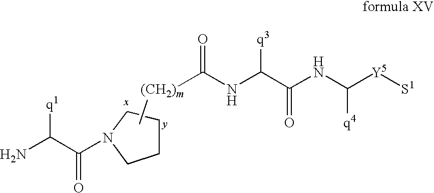

wherein:
   S¹, q¹, q³, q⁴, x and y are as defined above;
   m is an integer from 0 to 2; and
   Y⁵ is a linking moiety.

In some embodiments, the derivatives of endomorphin, or of endomorphin analogs are represented by formula XVI:

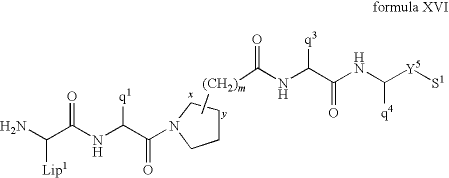

wherein:
   Lip¹, S¹, q¹, q³, q⁴, x and y are as defined above;
   m is an integer from 0 to 2; and
   Y⁵ is a linking moiety.

In some embodiments, the derivatives of endomorphin, or of endomorphin analogs are represented by formula XVII:

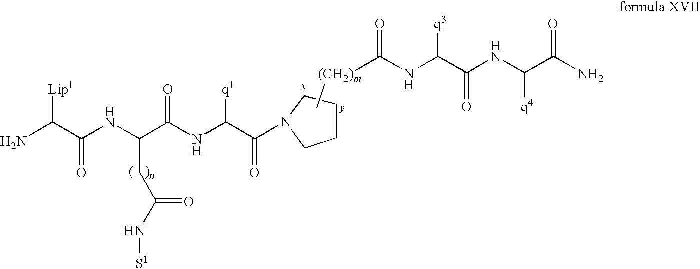

wherein:
   Lip¹, S¹, q¹, q³, q⁴, x and y are as defined above;
   m is an integer from 0 to 2; and
   n is an integer from 1 to 4.

In a further embodiment, the derivatives of endomorphin, or of endomorphin analogs are represented by formula XVIII,

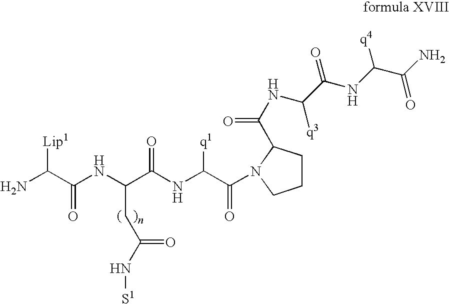

wherein:
   Lip¹ is C₆₋₁₀alkyl;
   n is an integer from 1 to 4;
   S¹ is a disaccharide;
   q¹ is selected from 4-hydroxybenzyl and 2,6-di-C₁₋₄alkyl-4-hydroxybenzyl;
   q³ is selected from any one of benzyl, α-methylbenzyl

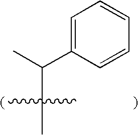

and 3-indolylmethyl; and
q⁴ is selected from benzyl and α-methylbenzyl.

In yet further embodiments, Lip¹ is octanyl and the disaccharide S¹ is selected from lactose, melibiose, cellobiose, isomaltose, maltose, allolactose and gentobiose.

In some embodiments, the lipo-, glyco- and glycolipid derivatives of endomorphin, and of endomorphin analogs, are fused with a heterologous polypeptide.

Suitably, the heterologous polypeptide is biologically active or has a carrier function.

In one aspect, the invention provides compounds represented by formula XIX:

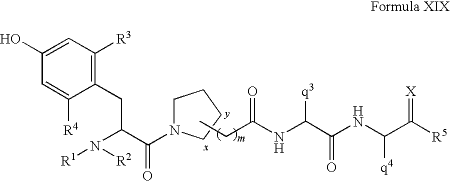

wherein:
   m is 0 or 1;
   x and y represent points of substitution on the pyrrolidine ring;
   q³ and q⁴ are each independently selected from an aryl, arylC₁₋₄alkyl, heteroaryl and heteroarylC₁₋₄alkyl group;
   R¹ and R² are each independently selected from hydrogen and —C(O)R⁶, or R¹ and R² taken together form ═CHR⁷;
      wherein:
      R⁶ is selected from C₅₋₂₀alkyl, C₅₋₂₀alkenyl, C₅₋₂₀alkynyl, —CH(NH₂)(C₄₋₁₉alkyl), —CH(NH₂)(C₄₋₁₉alkenyl), —CH(NH₂)(C₄₋₁₉alkynyl), —CH(NH₂)(aminoC₁₋₆alkyl), —CH(NH₂)(aminoC₂₋₆alkenyl), —CH(NH₂)(aminoC₂₋₆alkynyl),
      —CH(aminoC₁₋₆alkyONHC(O)(CH₂)ₜC(O)NHS¹,
      —CH(carboxyC₁₋₆alkyl)NHC(O)CH(NH₂)C₅₋₂₀alkyl,
      —CH(carboxyC₁₋₆alkyl)NHC(O)CH(NH₂)C₅₋₂₀alkenyl,
      —CH(carboxyC₁₋₆alkyl)NHC(O)CH(NH₂)C₅₋₂₀alkynyl,
      —CH(R⁸)(CH₂)_{q}C(O)NHS¹, —CH(NHC(O)R¹¹)(CH₂)NHC(O)R¹⁰,
      —CH(C₅₋₂₀alkyl)NHC(O)(CH₂)ₜC(O)NHS¹,
      —CH(C₅₋₂₀alkenyl)NHC(O)(CH₂)ₜC(O)NHS¹, and
      —CH(C₅₋₂₀alkynyl)NHC(O)(CH₂)ₜC(O)NHS¹,
         wherein:
         q is 1 to 4;
         u is 1 to 6;
         R⁸ is selected from hydrogen, —NH₂, —NHC(O)CH(NH₂)(C₅₋₂₀alkyl), —NHC(O)CH(NH₂)(C₅₋₂₀alkenyl), and
      —NHC(O)CH(NH₂)(C₅₋₂₀alkynyl);
         R¹⁰ is selected from C₅₋₂₀alkyl, C₅₋₂₀alkenyl, C₅₋₂₀alkynyl, —CH(NH₂)(C₄₋₁₉alkyl),
         —CH(NH₂)(C₄₋₁₉alkenyl), —CH(NH₂)(C₄₋₁₉alkynyl), and —(CH₂)ᵥC(O)NHS⁴,
         wherein:
         v is 2 to 4;
         R¹¹ is selected from —(CH₂)ₜC(O)NHS¹, C₅₋₂₀alkyl, C₅₋₂₀alkenyl, C₅₋₂₀alkynyl, —CH(NH₂)(C₄₋₁₉alkyl), —CH(NH₂)(C₄₋₁₉alkenyl), and —CH(NH₂)(C₄₋₁₉alkynyl);
         t is 2 to 4; and
         S¹ and S⁴ are each independently a cyclitol or saccharide;
      R⁷ is selected from C₅₋₂₀alkyl, C₅₋₂₀alkenyl, C₅₋₂₀alkynyl, —CH(NH₂)(C₄₋₁₉alkyl), —CH(NH₂)(C₄₋₁₉alkenyl), —CH(NH₂)(C₄₋₁₉alkynyl) and —CH(R⁸)(CH₂)ᵣC(O)NHS²;
         wherein:
         r is 1 or 2; and
         S² is a cyclitol or saccharide;
R³ and R⁴ are each independently selected from hydrogen, C₁₋₄alkyl and C₂₋₄alkenyl;
X is selected from O and N—R⁹;
   wherein:
      R⁹ is selected from hydrogen, C₅₋₂₀alkyl, C₅₋₂₀alkenyl, C₅₋₂₀alkynyl, —CH(C(O)NH₂)(C₄₋₁₉alkyl), —CH(C(O)NH₂)(C₄₋₁₉alkenyl), and —CH(C(O)NH₂)(C₄₋₄₉alkynyl);
R⁵ is selected from hydrogen, —NH₂, —NHS³, —NHCH(C(O)NH₂)(C₅₋₂₀alkyl), —NHCH(C(O)NH₂)(C₅₋₂₀alkenyl), —NHCH(C(O)NH₂)(C₅₋₂₀alkynyl), —NH(CO)CH(carboxyC₁₋₆alkyl)NHCH(C(O)NH₂)(C₅₋₂₀alkyl), —NH(CO)CH(carboxyC₁₋₆alkyl)NHCH(C(O)NH₂)(C₅₋₂₀alkenyl), and —NH(CO)CH(carboxyC₁₋₆alkyl)NHCH(C(O)NH₂)(C₅₋₂₀alkynyl);
   wherein:
   S³ is a cyclitol or saccharide.

Preferably, and where present, S¹, S² and S³ are each independently selected from the group consisting of: lactosyl, glucopyranosyl, mannopyranosyl, galactopyranosyl, 2-deoxy-2-acetamido-glucopyranosyl, 2-deoxy-2-acetamido-galactopyranosyl, maltosyl, glucoronyl, galacturonyl, melibiosyl, cellobiosyl, isomaltosyl, allolactosyl and gentobiosyl.

Preferably, q³ is selected from the group consisting of: 3-indolylmethyl, α-methylbenzyl, benzyl, naphthyl (eg 1-naphthyl) and biphenyl (eg 4-phenylphenyl).

Preferably, q⁴ is selected from the group consisting of: benzyl, α-methylbenzyl, toluoyl (eg 4-methylphenyl), xylyl (eg 2,4-dimethylphenyl), naphthyl (eg 1-naphthyl) and biphenyl (eg 4-phenylphenyl).

In a preferred embodiment of the invention, R² is hydrogen and R¹ is selected from the group consisting of: —C(O)CH(NH₂)(C₄₋₁₉alkyl), —C(O)CH(aminoC₃₋₅alkyl)NHC(O)(CH₂)₂C(O)NHS¹, —C(O)CH(NHC(O)(CH₂)₂C(O)NHS¹)(CH₂)₄NHC(O)CH(NH₂)(C₄₋₁₉alkyl), —C(O)CH(NHC(O)(CH₂)₂C(O)NHS¹)(CH₂)₄NHC(O)(CH₂)₂C(O)NHS¹, —C(O)CH(NHC(O)C₅₋₂₀alkyl)(CH₂)₄NHC(O)(CH₂)₂C(O)NHS¹, —C(O)CH(C₅₋₂₀alkyl)NHC(O)(CH₂)₂C(O)NHS¹, —C(O)CH(carboxyC₁₋₆alkyl)NHC(O)CH(NH₂)C₅₋₂₀alkyl, —C(O)CH₂(CH₂)_{q}C(O)NHS¹, —C(O)CH(NH₂)(CH₂)_{q}C(O)NHS¹ and —C(O)CH(NHC(O)CH(NH₂)(C₅₋₂₀alkyl))(CH₂)_{q}C(O)NHS¹, wherein q is 1 to 4, and S¹ is a saccharide or cyclitol. Even more preferably, R² is hydrogen and R¹ is selected from the group consisting of: —C(O)CH(NH₂)(C₅₋₁₈alkyl), —C(O)CH(aminobutyl)NHC(O)(CH₂)₂C(O)NHS¹, —C(O)CH(NHC(O)(CH₂)₂C(O)NHS¹)(CH₂)₄NHC(O)CH(NH₂)(C₅₋₁₂alkyl), —C(O)CH(NHC(O)(CH₂)₂C(O)NHS¹)(CH₂)₄NHC(O)(CH₂)₂C(O)NHS¹, —C(O)CH(NHC(O)C₅₋₁₂alkyl)(CH₂)₄NHC(O)(CH₂)₂C(O)NHS¹, —C(O)CH(C₅₋₁₂alkyl)NHC(O)(CH₂)₂C(O)NHS¹, —C(O)CH(carboxyC₁₋₂alkyl)NHC(O)CH(NH₂)C₅₋₁₂alkyl, —C(O)CH₂(CH₂)_{q}C(O)NHS¹, —C(O)CH(NH₂)(CH₂)_{q}C(O)NHS¹ and —C(O)CH(NHC(O)CH(NH₂)(C₅₋₁₂alkyl))(CH₂)_{q}C(O)NHS¹, wherein q is 1 to 3, and S¹ is a saccharide or cyclitol.

In another preferred embodiment of the invention, R¹ and R² together form ═CHCH(NH₂)(C₄₋₁₉alkyl), more preferably, R¹ and R² together form ═CHCH(NH₂)(C₅₋₁₂alkyl).

In another preferred embodiment of the invention, R¹ and R² are each hydrogen.

Preferably, R³ and R⁴ are each independently selected from hydrogen and C₁₋₂ alkyl.

Preferably, X is selected from oxygen and ═NCH(C(O)NH₂)(C₅₋₁₂alkyl).

Preferably, R⁵ is selected from the group consisting of: hydrogen, —NH₂, —NHS³, —NHCH(C(O)NH₂)(C₅₋₁₂alkyl) and —NH(CO)CH(carboxyC₁₋₂alkyl)NHCH(C(O)NH₂)(C₅₋₁₂alkyl), wherein S³ is a saccharide or cyclitol.

Another aspect of the present invention provides methods for agonising a μ-opioid receptor. These methods generally comprise contacting a μ-opioid receptor with a derivative as broadly described above.

Modifications to the peptide backbone and to peptide bonds thereof are encompassed within the scope of amino acid analogs contemplated by the present invention (the resulting peptide being a peptide analog). As used herein, an “amino acid analog” is an organic molecule that is a pharmacophore of an amino acid (native amino acid), and has a bio-isosteric group or side chain corresponding to that group or side chain present in the native amino acid of which it is an analog. A “peptide analog,” as used herein, is an organic molecule that is a pharmacophore of a peptide. A peptide analog exhibits substantially the same spatial arrangement between adjacent functional groups or side chains as is found in the native peptide of which it is an analog. Modifications can be made to the amino acid, derivative thereof, non-amino acid moiety or the peptide, either before or after the amino acid, derivative thereof or non-amino acid moiety is incorporated into the peptide. Such modifications mimic the peptide backbone and bonds, which make up the same and have substantially the same or similar spacial arrangement and distance as is typical for traditional peptide bonds and backbones. An example of one such modification is the reduction of the carbonyl(s) of the amide peptide backbone to an amine. A number of reagents are available and well known for the reduction of amides to amines such as those disclosed in Wann et al., Journal of Organic Chemistry, 1981, 46, 257 and Raucher et al., Tetrahedron Letters, 1980, 21, 14061.

The substitution of amino acids by non-naturally occurring amino acids and amino acid analogs as described above can enhance the overall activity or properties of an individual peptide thereof based on the modifications to the backbone or side chain functionalities. For example, these types of alterations can enhance the peptide's stability to enzymatic breakdown and increase biological activity. Modifications to the peptide backbone similarly can add stability and enhance activity.

Endomorphin mimetics such as peptidomimetics may also be modified to improve physicochemical properties, such as cell permeability and stability, through conjugation of one or more lipidic or saccharidic moieties. Illustrative examples of endomorphin peptidomimetics can be found, for example, in WO 2004/033414.

Where an amino acid residue in a peptide is recognised as having structural as opposed to functional significance in the peptide chain, that amino acid residue may be replaced by an alternative chemical moiety such that the overall functional activity of the peptide is maintained. For example, a residue may be replaced by an alternative chemical moiety or linking functionality such that the activity of the overall peptide is conserved or enhanced, the peptide's stability enhanced, or the peptide's bioavailability modified.

In some embodiments, the lipidic group or lipid amino acid group is attached to an endomorphin or an endomorphin analog through a “pro-drug” linkage.

Suitably, the compounds of the present invention may be purified by chromatographic techniques including high-performance liquid chromatography (HPLC) and reverse phase HPLC.

The compounds of the present invention may be prepared using methods analogous to those described in the prior art.

Combinatorial methods may be employed, using solid or solution phase techniques known to the art, to prepare libraries of compounds.

In general, techniques for preparing peptides are well known in the art for example see Alewood, P.; Alewood, D.; Miranda, L.; Love, S.; Meutermans, W.; Wilson, D., “Rapid in situ neutralisation protocols for Boc and Fmoc solid-phase chemistries”. Methods in Enzymology 1997, 289, 14-28; Merrifield, Journal of the American Chemical Society 1964, 85, 2149; Bodanzsky, “Principles of Peptide Synthesis”, 2nd Ed., Springer-Verlag (1993)); and Houghten, Proceedings of the National Academy of Sciences USA, 1985, 82, 5131.

Amino acid derivatives and amino acid bio-isosteres can be prepared using methods analogous to those described in the art. For example, optically pure 2′,6′-dimethyl-L-tyrosine (Dmt) can be prepared according to the methods described in Dygos J. H., et al, Synthesis, 1992, 8, 741. Referring to Scheme 1 below, other tyrosine analogs can be prepared according to the methods described in Li, T., Journal of Medicinal Chemistry, 2005, 48, 586-592. As shown in Scheme 1, a mono-, di- or tri-substituted phenol may be iodinated in the 4-position of the aromatic ring (Scheme 1 (i)), the phenolic hydroxy group then protected as an ester (Scheme 1 (ii)), followed by Heck-type reaction (Scheme 1 (iii)), subsequent reduction (Scheme 1 (iv)) and deprotection (Scheme 1 (v)) to afford a range of tyrosine amino acid derivatives. For example, R^{a}, R^{b} and R^{c} can be selected from H and C₁₋₄alkyl groups. Subsequently, the carboxyl or amino functional groups can be protected, if required, for further synthetic steps For example Boc protection of the amino function by reaction with (Boc)₂O in the presence of triethylamine (Scheme 1 (vi)) affords the monoprotected derivatives suitable for peptide syntheses.

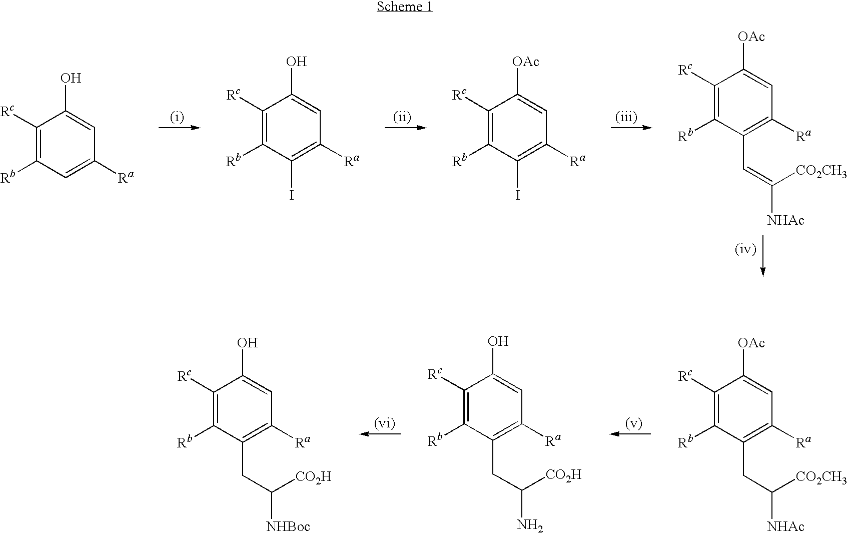

In some embodiments, a lipid, in the form of a lipo-amino acid may be conjugated to the N-terminus of an endomorphin or endomorphin analog, followed by subsequent conjugation of a sugar residue. Referring to Scheme 2 below, an amino acid residue, with a q⁴ side chain is conjugated to a solid support using solid phase peptide synthesis methods. Herein, a q⁴ side chain, is a side chain that is bio-isosteric with the side chain of phenylalanine. A second amino acid residue with a q³ side chain is subsequently coupled to the initial resin bound amino acid residue. Herein, a q³ side chain, is a side chain that is bio-isosteric with the side chain of either phenylalanine or tryptophan. A lipo-amino acid residue is then conjugated to the support. The chain length of Lip¹ is typically from about 6 to about 22 carbon atoms in length. An amino acid residue with a q¹ side chain is then coupled to the growing resin bound molecule. Herein, a q¹ side chain, is a side chain that is bio-isosteric with the side chain of tyrosine. Finally, a monosaccharide moiety is coupled to the resin bound molecule via, as shown in this example, a urea linkage. Other suitable linkages will be known to those skilled in the art. The molecule is then cleaved from the solid support to afford a sugar-lipid-endomorphin analog. By varying q¹, q³ and q⁴ is it possible to prepare combinatorial libraries based on the structure of A.

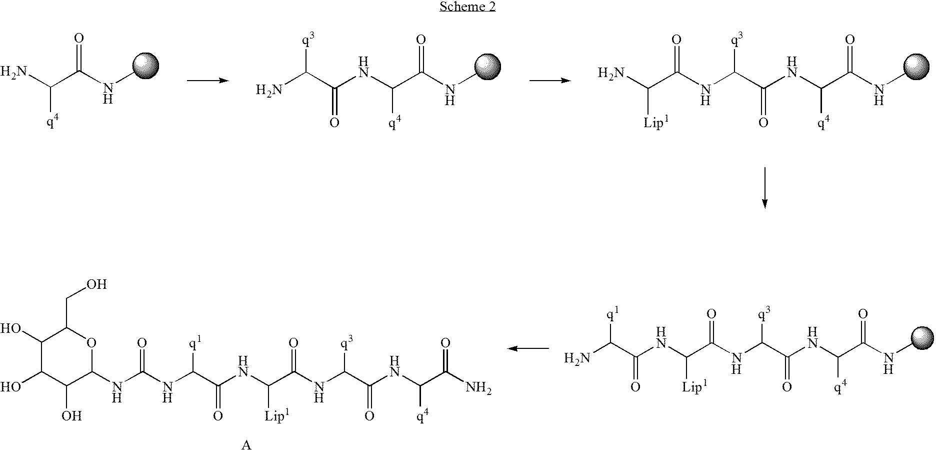

In some embodiments, a lipid, in the form of a lipo-amino acid may be conjugated to the N-terminus of an endomorphin or endomorphin analog. In the endomorphin analogs, the proline residue may be replaced with structural equivalents, or other chemical moieties that modulate the pharmacological activity, stability, and/or structure of the endomorphin analogs. Referring to Scheme 3 below, sequentially, amino acid residues with q⁴ and q³ side chains are conjugated to a solid support using solid phase peptide synthesis methods. A moiety P¹ is then coupled to the resin bound chain. Subsequently, an amino acid moiety with a q¹ side chain can be coupled to the resin bound chain followed by a lipo-amino acid. The molecule can then be cleaved from the resin to form compound B.

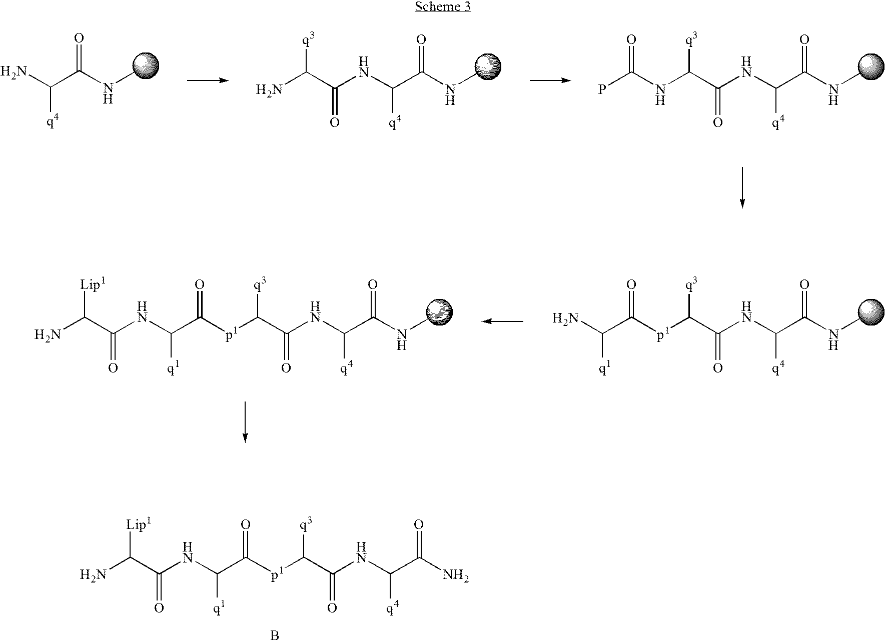

Without wishing to be bound by theory it is believed that position 1 of the tetrapeptide—tyrosine (Tyr), position 3—tryptophan (Trp) (for endomorphin 1) or phenylalanine (Phe) (for endomorphin-2), and position 4—phenylalanine (Phe), are involved in hydrogen bonding and hydrophobic interactions with the receptor, while position 2—proline (Pro) may have a role as a spacer that induces the other residues to assume the proper spatial orientation for the ligand-receptor complex formation. Substitutions of the α-proline residue with other amino acid residues have been disclosed, for example in: beta-prolines: Spampinato, S., et al, European Journal of Pharmacology, 2003, 469, 89-95; Cardillo, G., et al, Bioorganic & Medicinal Chemistry Letters, 2000, 10, 2755-2758; Cardillo, G., et al, Journal of Medicinal Chemistry, 2002, 45, 2571-2578; Van den Eynde et al, Journal of Medicinal Chemistry, 2005, 48, 3644-3648; Balboni, G., et al, Journal of Medicinal Chemistry, 2005, 48, 5608-5611; Kruszynski, R., Bioorganic & Medicinal Chemistry Letters, 2005, 13, 6713-6717; and Sperling a, E., et al, Bioorganic & Medicinal Chemistry Letters, 2005, 15, 2467-2469.

In some embodiments, a lipid, in the form of a lipo-amino acid, may be conjugated to the C-terminus of an endomorphin or endomorphin analog. Referring to Scheme 4 below, a lipo-amino acid residue with a Lip¹ side chain, and which is bound to a solid support, is subsequently coupled sequentially with amino acid residues with q⁴ and q³ side chains. The resin bound chain is subsequently coupled with a moiety P¹ and finally by an amino acid residue with a q¹ side chain. The molecule is then cleaved from the support to provide an endomorphin analog C with a lipid moiety coupled to the C-terminus.

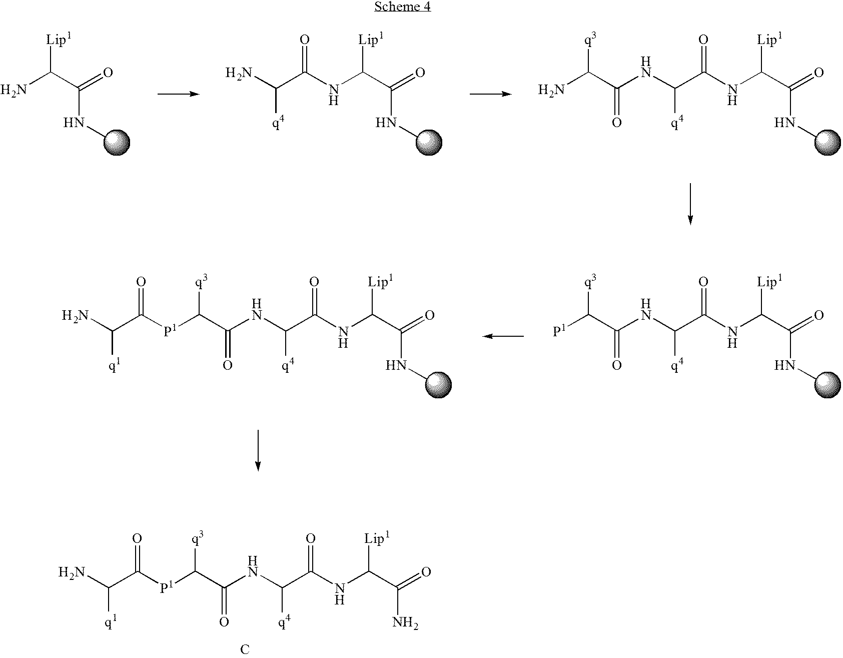

In some embodiments, the moiety P¹ may be functionalised, for example with a sugar moiety. Referring to Scheme 5 below, sequentially, amino acid residues with q⁴ and q³ side chains are conjugated to a solid support using solid phase peptide synthesis methods. A 4-hydroxyproline moiety, derivatised with a disaccharide (DiS) through the 4-hydroxyl group of proline, is then coupled to the resin bound chain. Other polyhydroxylated moieties such as monosaccharides and cyclitols may also be conjugated through the 4-hydroxyl group of proline. Further, it is envisioned that a spacer may be employed between the proline hydroxy group and the sugar moiety. Furthermore, the hydroxyl group may be converted through known chemistries to another functional group such as an amino group. To the proline derivative residue may be coupled an amino acid residue with a q¹ side chain. Subsequently a lipo-amino acid residue with a Lip¹ side chain may be coupled to the resin bound molecule. The molecule is then cleaved from the resin affording D. The molecule can be deprotected in solution phase or on solid phase as appropriate. Protecting group chemistries for the protection of sugar functional group and functional groups of peptidic side chains are well known in the art and may be found for example in: Theodora W. Greene and Peter G. M. Wuts, Protecting Groups in Organic Synthesis, (Third Edition, John Wiley & Sons, Inc, 1999).

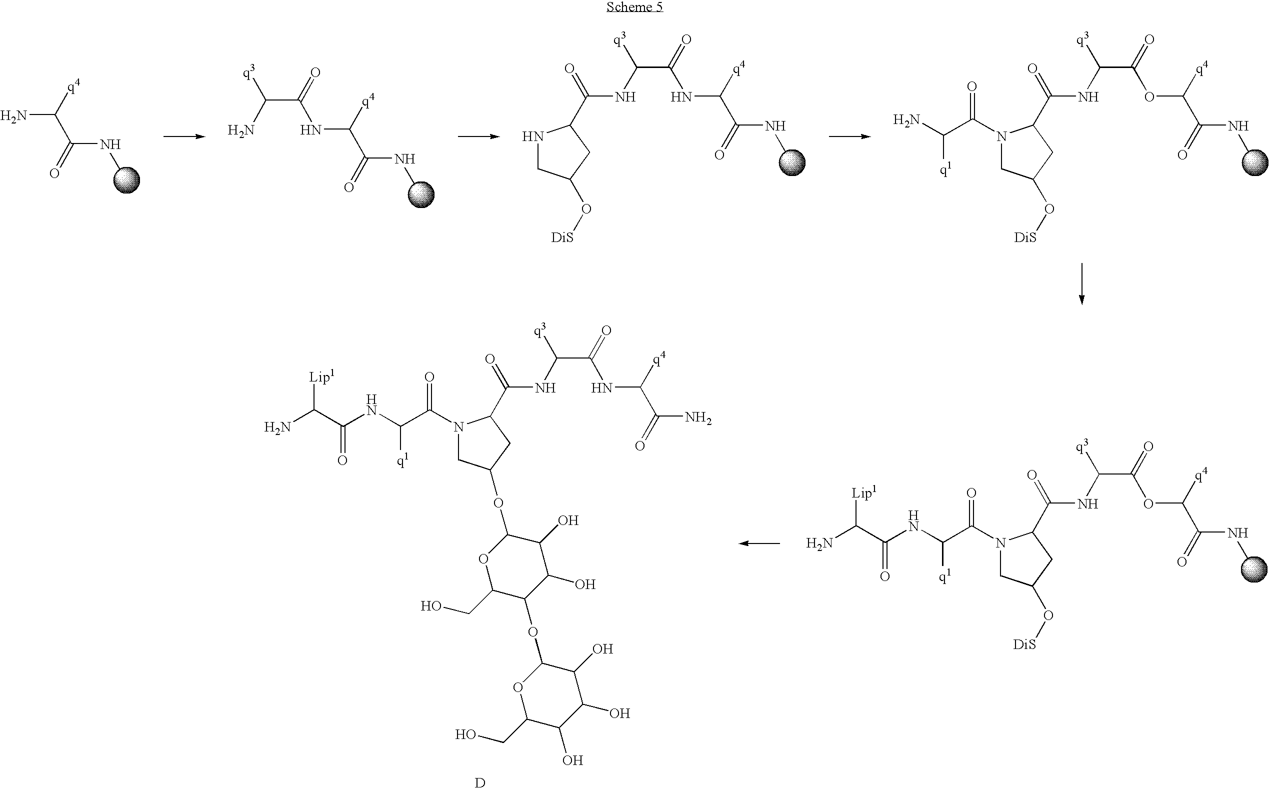

In some embodiments, the moiety P¹ may be a D- or L-, alpha- or beta-proline moiety.

Referring to Scheme 6 below, amino acid residues with q⁴ and q³ side chains are sequentially conjugated to a solid support using solid phase peptide synthesis methods. An alpha- or beta-proline is then coupled to the resin bound chain. In Scheme 6 below, “x” and “y” refer to points of substitution on the pyrrolidine residue. Preferably, when the point of connection is “x”, then m=0 or 1, and when the point of connection is “y”, m=0. The proline residue may be the natural L isomer or the unnatural D isomer. After coupling of the proline residue, an amino acid residue with a q¹ side chain is coupled to the resin bound chain followed by an amino acid to which is conjugated a sugar moiety S¹, and followed finally by a lipo-amino acid with a Lip¹ side chain. The S¹ side chain refers to a sugar moiety which is attached via a linking moiety to the peptidic backbone. A sugar moiety may be attached, for example, through an aspartic acid, glutamic acid or lysine side chain. As such, a glycosyl amine may be reacted with the carboxylate function of the side chain of a suitably protected aspartic or glutamic acid residue, or reacted with the amine function of the side chain of a suitably protected lysine residue, under conditions known in the art, to form the desired sugar conjugate. Alternatively, the resin bound molecule may be cleaved from the solid support prior to the coupling of the lipo-amino acid in order to form a sugar endomorphin conjugate G.

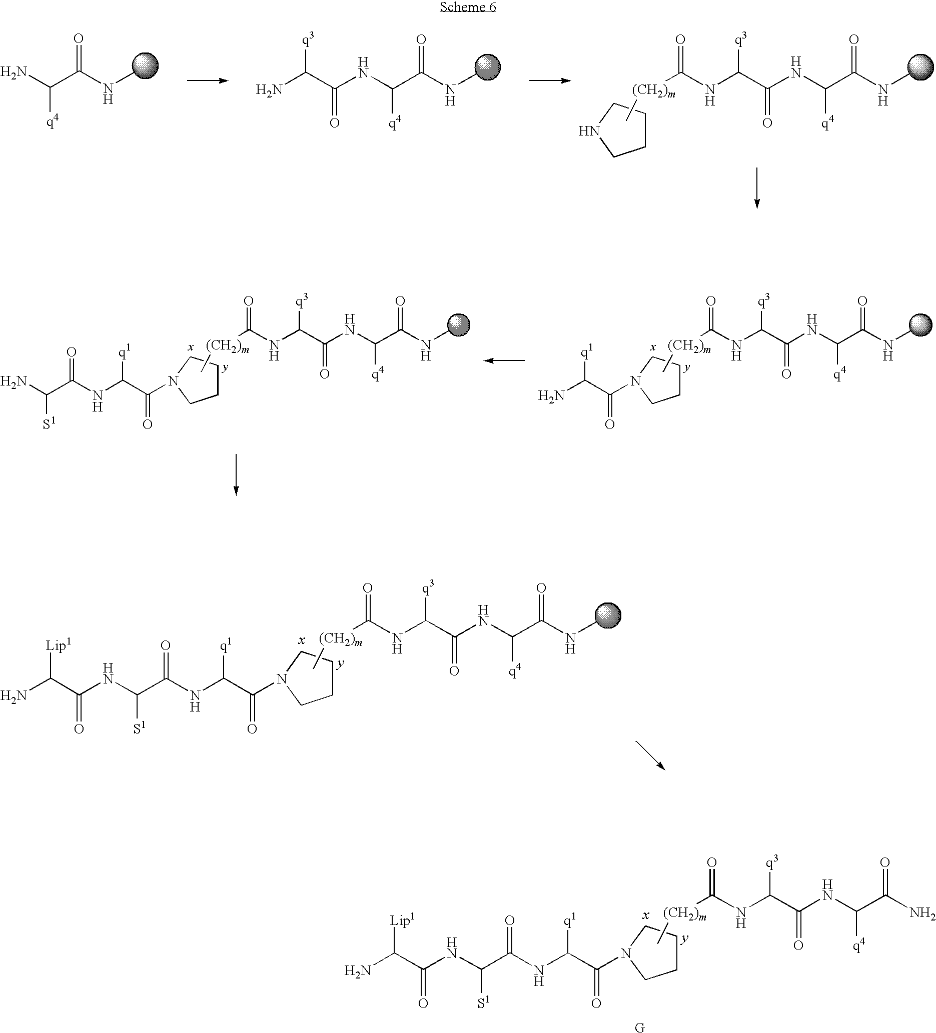

In some embodiments, the lipid moiety can be conjugated to an endomorphin or endomorphin analog via a pro-drug linkage. For example, and referring to Scheme 7 below, after sequential conjugation of amino acid residues with q⁴ and q³ side chains, an alpha- or beta-proline, and an amino acid residue with a q¹ side chain, an aldehyde can be reacted with the terminal amino function to form an imine (H). Alternatively, an amino acid to which is conjugated a carbohydrate moiety, can be conjugated to the amino acid residue with the q¹ side chain and similarly an alkanal can be reacted with the corresponding amino function affording I. Other suitable pro-drug linkages are known to those skilled in the art.

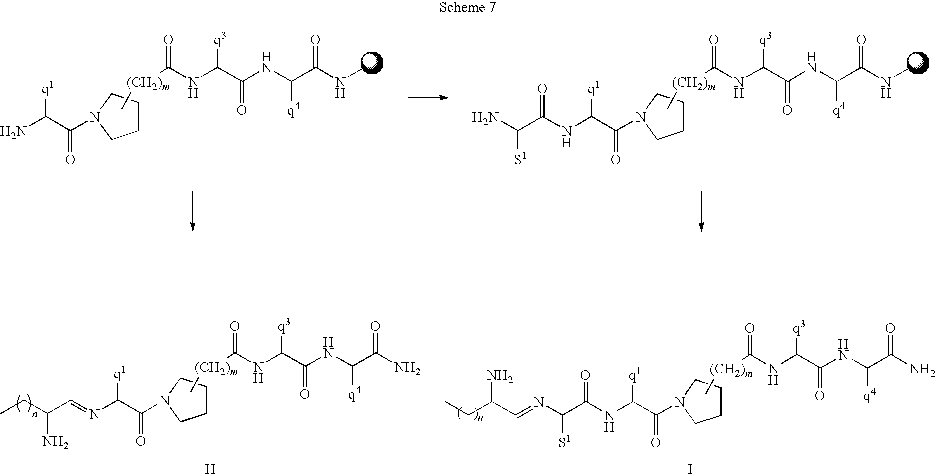

In some embodiments, a spacer may be employed between an endomorphin or endomorphin analog and the lipid or carbohydrate moiety. For example, and referring to Scheme 8 below, a succinoyl moiety, or an amino acid such as lysine, aspartic acid or glutamic acid, can be used as a spacer to bridge between the endomorphin or endomorphin analog and the lipid or carbohydrate moiety (compounds J and K). Other moieties suitable as spacers, for example, polyethylene glycol moieties, di-functionalised alkyl chains such as glutarates, and adipates, are known to those skilled in the art. Referring to Scheme 9 below, a spacer may be used as a linking group between an endomorphin analog and the lipid moiety, or, for example, a spacer may be used between a lipid moiety such as a lipo-amino acid and a moiety bearing a carbohydrate attached to the endomorphin analog (L).

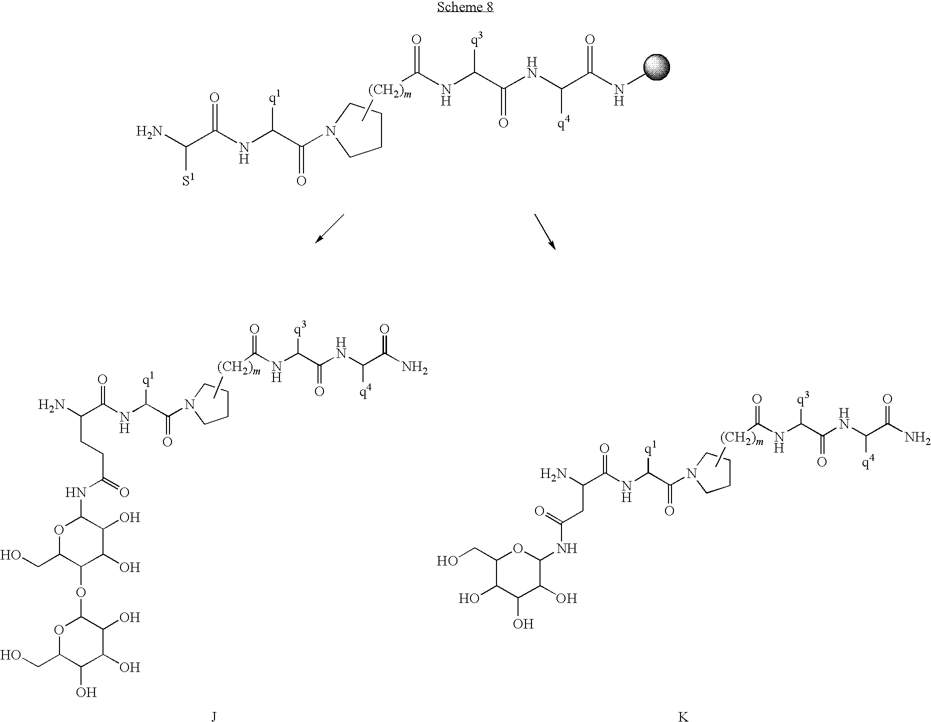

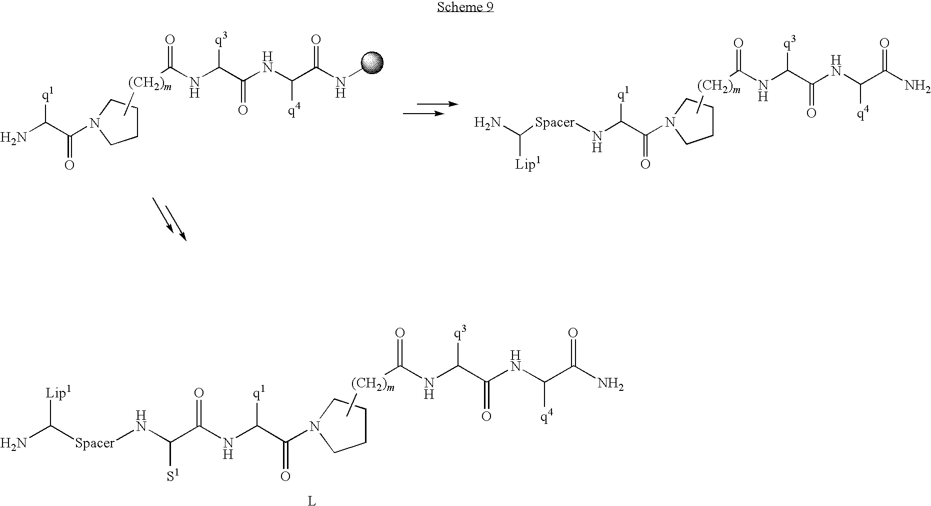

Referring to Scheme 10 below, in some embodiments, the conjugate may have a lipid moiety connected to the N-terminus and a carbohydrate moiety conjugated to the C-terminus (M). It is understood that equally the conjugate may comprise a lipid moiety conjugated to the C-terminus and a carbohydrate moiety conjugated to the N-terminus. As previously mentioned, it is also understood that both solid phase and solution phase peptide synthesis methods are suitable for the purposes of the present invention. In Scheme 10 below, T¹ refers to a temporary amino protecting group.

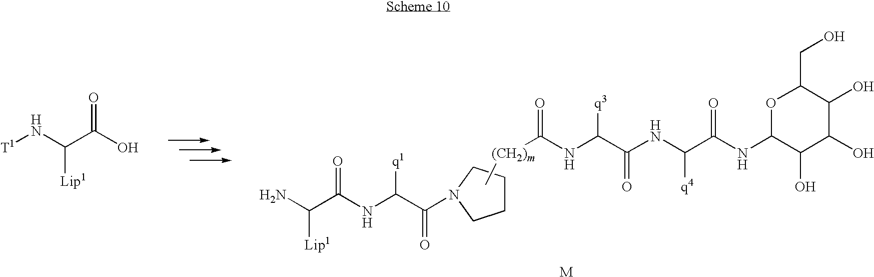

Referring to Scheme 11 below, in some embodiments a carbohydrate moiety may be attached via an amide linkage directly to an endomorphin or an endomorphin analog (N).

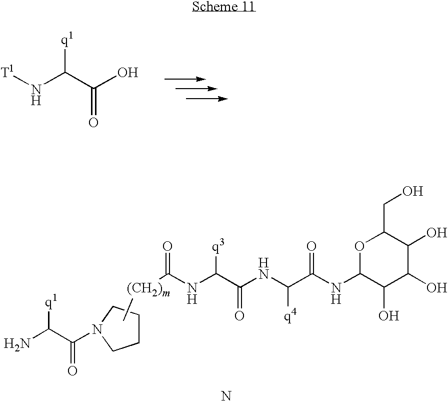

In some embodiments, more than one lipid moiety may be conjugated to an endomorphin or endomorphin analog. For example, and referring to Scheme 12 below, an amino acid to which is conjugated a carbohydrate moiety, can be conjugated to the amino acid residue containing a q¹ side chain, and subsequently two further lipo-amino acid residues with side chains Lip¹ may be sequentially coupled to form a conjugate with two lipid groups (O). It is understood that the order of conjugation of, the amino acid to which is conjugated a carbohydrate moiety, and, the lipo-amino acids, is not necessarily fixed. For example, a lipo-amino acid may be conjugated to an endomorphin analog followed by an amino acid to which is conjugated a carbohydrate moiety, followed by a further lipo-amino acid. Alternatively, two lipo-amino acids may be sequentially conjugated to an endomorphin analog followed subsequently by conjugation of an amino acid to which is coupled a carbohydrate moiety.

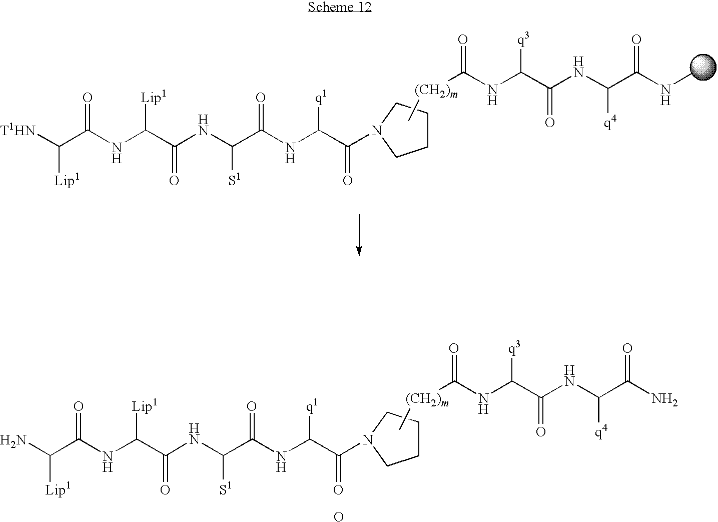

Further examples of specific compounds included within the scope of the invention are shown in Table 2 below.

TABLE 2
Selected Compounds

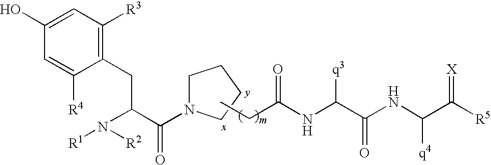

| # | X | m | x^{*} | y^{*} | q³ | q² | R¹ | R² | R³ | R⁴ | R⁵ |
|---|---|---|---|---|---|---|---|---|---|---|---|
| Endol | O | 0 | + | | 3- | benzyl | H | H | H | H | NH₂ |
| | | | | | indolyl- | | | | | | |
| | | | | | methyl | | | | | | |
| (Dmt¹) | O | 0 | + | | 3- | benzyl | H | H | Me | Me | NH₂ |
| Endol | | | | | indolyl- | | | | | | |
| | | | | | methyl | | | | | | |
| 1 | O | 0 | + | | 3- | benzyl | 2-aminooctanoyl | H | H | H | NH₂ |
| | | | | | indolyl- | | | | | | |
| | | | | | methyl | | | | | | |
| 2 | O | 0 | + | | 3- | benzyl | 2-aminododecanoyl | H | H | H | NH₂ |
| | | | | | indolyl- | | | | | | |
| | | | | | methyl | | | | | | |
| 3 | O | 0 | + | | 3- | benzyl | 2-aminodecanoyl | H | H | H | NH₂ |
| | | | | | indolyl- | | | | | | |
| | | | | | methyl | | | | | | |
| 4 | O | 0 | + | | 3- indolyl- methyl | benzyl | 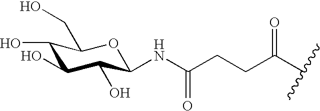 | H | H | H | NH₂ |
| 5 | O | 0 | + | | 3- indolyl- methyl | benzyl | 2-aminooctanoyl | H | H | H | 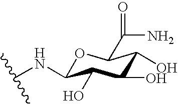 |
| 6 | O | 0 | + | | 3- indolyl- methyl | benzyl | 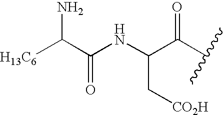 | H | H | H | NH₂ |
| 7 | O | 0 | + | | 3- | benzyl | 2- | H | Me | Me | NH₂ |
| | | | | | indolyl- | | amino- | | | | |
| | | | | | methyl | | octanoyl | | | | |
| 8 | O | 0 | + | | 3- | benzyl | 2- | H | Me | Me | NH₂ |
| | | | | | indolyl- | | amino- | | | | |
| | | | | | methyl | | decanoyl | | | | |
| 9 | O | 0 | + | | 3- | benzyl | 2- | H | Me | Me | NH₂ |
| | | | | | indolyl- | | amino- | | | | |
| | | | | | methyl | | dodecanoyl | | | | |
| 10 | O | 0 | + | | 3- indolyl- methyl | benzyl | 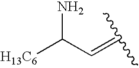 | | H | H | NH₂ |
| 11 | O | 0 | + | | 3- indolyl- methyl | benzyl | 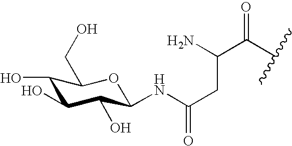 | H | H | H | NH₂ |
| 12 | O | 0 | + | | 3- indolyl- methyl | benzyl | 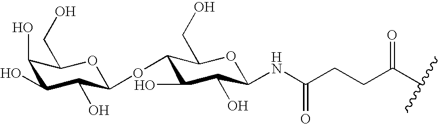 | H | H | H | NH₂ |
| 13 | O | 0 | + | | 3- indolyl- methyl | benzyl | 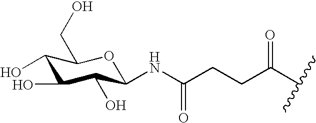 | H | H | H | 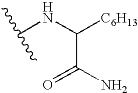 |
| 14 | O | 0 | + | | 3- indolyl- methyl | benzyl | 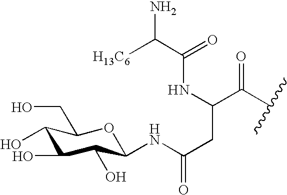 | H | H | H | NH₂ |
| 15 | O | 0 | + | | 3- indolyl- methyl | benzyl | 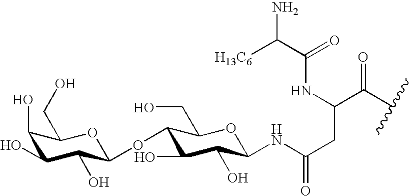 | H | H | H | NH₂ |
| 16 | O | 0 | + | | 3- indolyl- methyl | benzyl | H | H | H | H | 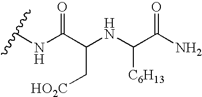 |
| 17 | 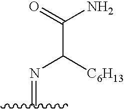 | 0 | + | | 3- indolyl- methyl | benzyl | H | H | H | H | H |
| 18 | O | 0 | + | | 3- indolyl- methyl | benzyl | H | H | H | H | 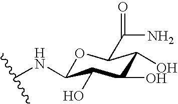 |
| 19 | O | 0 | + | | 3- indolyl- methyl | benzyl | H | H | H | H | 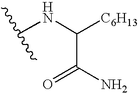 |
| 20 | O | 0 | + | | 3- indolyl- methyl | benzyl | 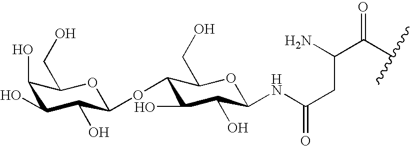 | H | H | H | NH₂ |
| 21 | O | 0 | + | | 3- indolyl- methyl | benzyl | 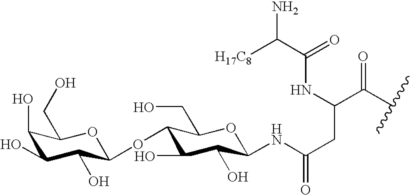 | H | Me | Me | NH₂ |
| 22 | O | 0 | + | | 3- indolyl- methyl | benzyl | 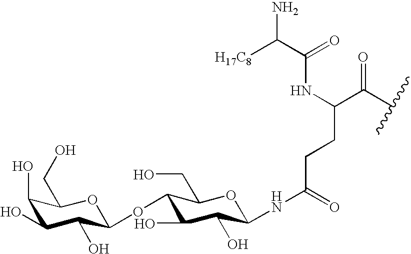 | H | Me | Me | NH₂ |
| 23 | O | 0 | | + | α- methyl- benzyl | benzyl | 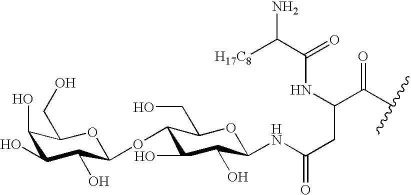 | H | Me | Et | NH₂ |
| 24 | O | 0 | + | | benzyl | benzyl | 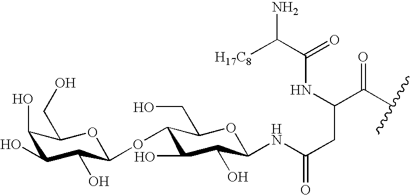 | H | Me | Me | NH₂ |
| 25 | O | 0 | + | | α- methyl- benzyl | benzyl | 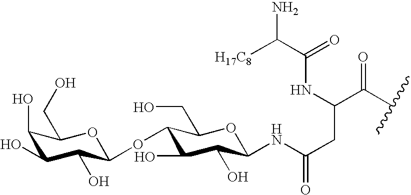 | H | H | H | NH₂ |
| 26 | O | 0 | | + | 3- indolyl- methyl | benzyl | 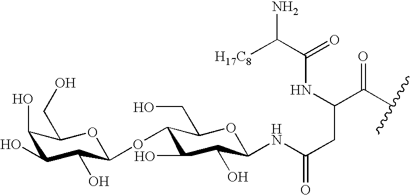 | H | Me | Me | NH₂ |
| 27 | O | 1 | + | | 3- indolyl- methyl | benzyl | 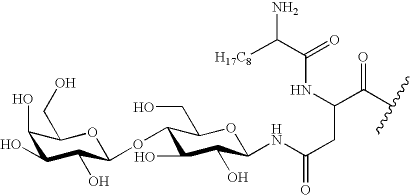 | H | Me | Me | NH₂ |
| 28 | O | 0 | + | | benzyl | α- methyl- benzyl | 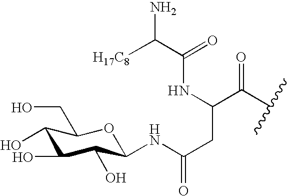 | H | Me | Me | NH₂ |
| 29 | O | 0 | + | | α- methyl- benzyl | benzyl | 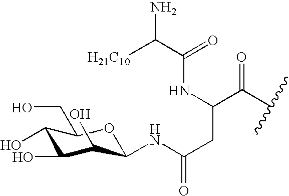 | H | Me | Me | NH₂ |
| 30 | O | 0 | + | | naphthyl | benzyl | 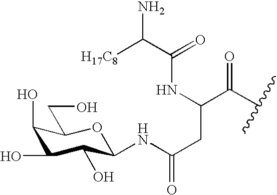 | H | Me | Me | NH₂ |
| 31 | O | 0 | + | | biphenyl | toluyl | 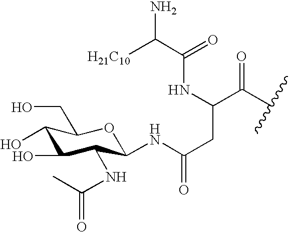 | H | Me | Me | NH₂ |
| 32 | O | 0 | + | | 3- indolyl- methyl | xylyl | 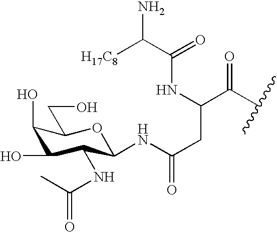 | H | Me | Me | NH₂ |
| 33 | O | 0 | + | | benzyl | benzyl | 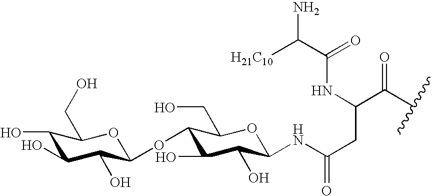 | H | Me | Me | NH₂ |
| 34 | O | 0 | + | | α- methyl- benzyl | naphthyl | 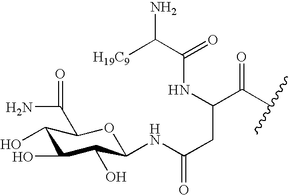 | H | Me | Me | NH₂ |
| 35 | O | 0 | | + | naphthyl | biphenyl | 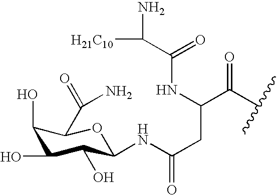 | H | Me | Me | NH₂ |
| 36 | O | 0 | | + | biphenyl | benzyl | 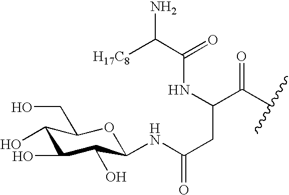 | H | H | H | NH₂ |
| 37 | O | 0 | | + | 3- indolyl- methyl | benzyl | 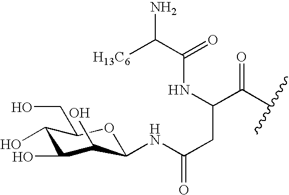 | H | H | H | NH₂ |
| 38 | O | 0 | | + | benzyl | benzyl | 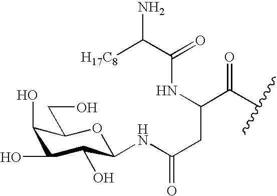 | H | H | H | NH₂ |
| 39 | O | 0 | | + | α- methyl- benzyl | toluyl | 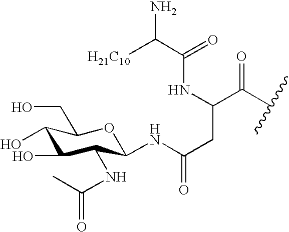 | H | H | H | NH₂ |
| 40 | O | 0 | + | | naphthyl | benzyl | 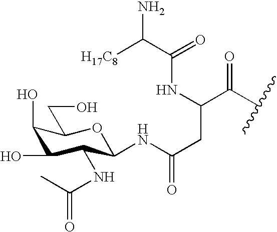 | H | H | H | NH₂ |
| 41 | O | 0 | + | | 3- indolyl- methyl | benzyl | 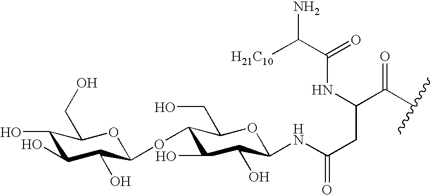 | H | H | H | NH₂ |
| 42 | O | 0 | | + | 3- indolyl- methyl | benzyl | 2-aminooctadecanoyl | H | H | H | NH₂ |
| 43 | O | 0 | + | | 3- indolyl- methyl | benzyl | H | H | H | H | 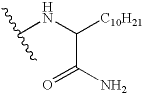 |
| 44 | O | 0 | + | | 3- indolyl- methyl | benzyl | 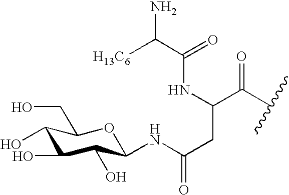 | H | H | H | NH₂ |
| 45 | O | 0 | + | | 3- indolyl- methyl | benzyl | 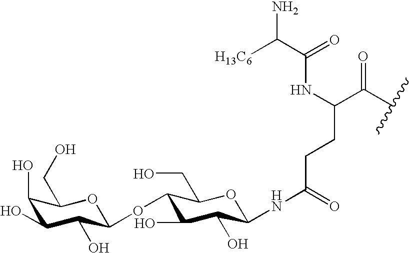 | H | H | H | NH₂ |
| 46 | O | 0 | + | | 3- indolyl- methyl | benzyl | 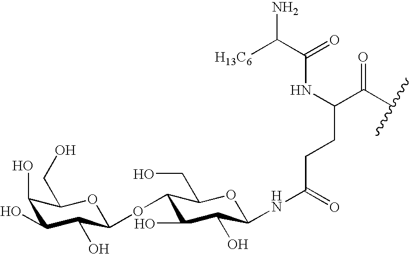 | H | Me | Me | NH₂ |
| 47 | O | 0 | + | | 3- indolyl- methyl | benzyl | 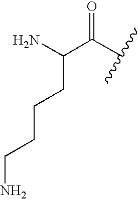 | H | H | H | NH₂ |
| 48 | O | 0 | + | | 3- indolyl- methyl | benzyl | 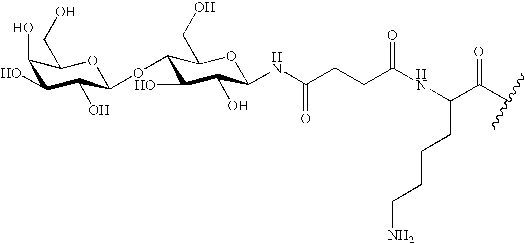 | H | H | H | NH₂ |
| 49 | O | 0 | + | | 3- indolyl- methyl | benzyl | 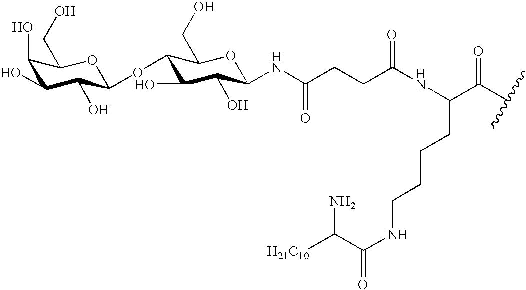 | H | H | H | NH₂ |
| 50 | O | 0 | + | | 3- indolyl- methyl | benzyl | 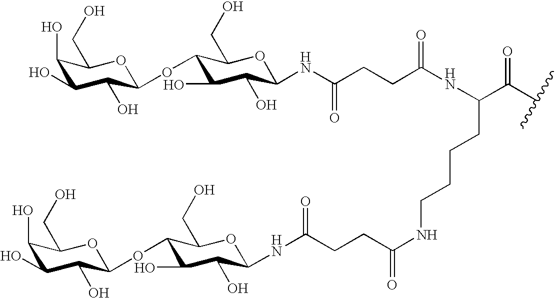 | H | H | H | NH₂ |
| 51 | O | 0 | + | | 3- indolyl- methyl | benzyl | 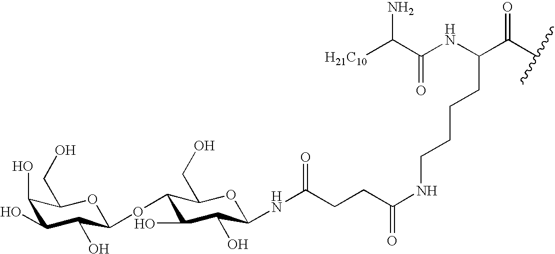 | H | H | H | NH₂ |
| 52 | O | 0 | + | | 3- indolyl- methyl | benzyl | 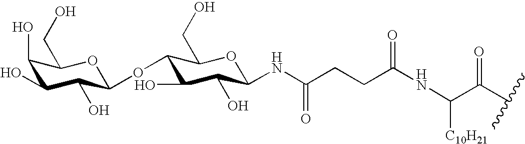 | H | H | H | NH₂ |
| 53 | O | 0 | + | | 3- indolyl- methyl | benzyl | 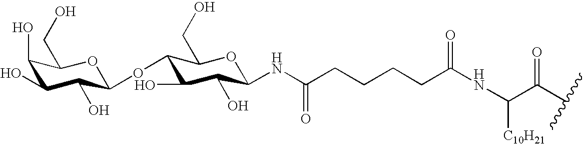 | H | H | H | NH₂ |
| 54 | O | 0 | + | | 3- indolyl- methyl | benzyl | 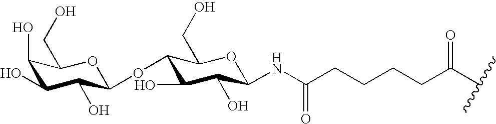 | H | H | H | NH₂ |

^{*}A ‘+’ sign indicates a point of substitution on the pyrrolidine ring

Other examples of compounds provided by the present invention include the following:

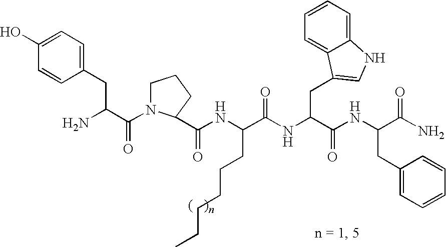

### 3. Compositions

Another aspect of the present invention provides compositions for producing analgesia and for treating, preventing and/or relieving the symptoms of pain, comprising an effective amount of a derivative as broadly described in Section 2 and a pharmaceutically acceptable carrier and/or diluent.

Any derivative as broadly described above can be used in the compositions and methods of the present invention, provided that it is pharmaceutically active. A “pharmaceutically active” derivative is in a form which results in the treatment and/or prevention of pain, including the prevention of incurring a symptom, holding in check such symptoms or treating existing symptoms associated with pain, when administered to an individual.

The effect of compositions of the present invention may be examined by using one or more of the published models of pain/nociception. The analgesic activity of the compounds of this invention can be evaluated by any method known in the art. Examples of such methods include the Tail-flick test (D'Amour et al. 1941, J. Pharmacol. Exp. and Ther. 72: 74-79); the Rat Tail Immersion Model, the Carrageenan-induced Paw Hyperalgesia Model, the Formalin Behavioral Response Model (Dubuisson et al., 1977, Pain 4: 161-174), the Von Frey Filament Test (Kim et al., 1992, Pain 50: 355-363), the Radiant Heat Model, the Cold Allodynia Model (Gogas et al., 1997, Analgesia 3: 111-118), the paw pressure test (Randall and Selitto, 1957, Arch Int Pharmacodyn 111: 409-419) and the paw thermal test (Hargreaves et al., 1998, Pain 32: 77-88). Derivatives of the present invention, which test positive in such assays, are particularly useful for the prevention, reduction, or reversal of pain in a variety of pain-associated conditions or pathologies.

The pharmaceutically active derivatives of the present invention may be provided as salts with pharmaceutically compatible counterions. Pharmaceutically compatible salts may be formed with numerous acids or bases. Examples of suitable acids include but not limited to hydrochloric, sulfuric, acetic, lactic, tartaric, malic, succinic acid. Salts tend to be more soluble in aqueous or other protonic solvents that are the corresponding free base forms.

Pharmaceutical compositions suitable for use in the present invention include compositions wherein the pharmaceutically active compounds are contained in an effective amount to achieve their intended purpose. The dose of active compounds administered to a patient should be sufficient to achieve a beneficial response in the patient over time such as a reduction in, or relief from, pain. The quantity of the pharmaceutically active compounds(s) to be administered may depend on the subject to be treated inclusive of the age, sex, weight and general health condition thereof. In this regard, precise amounts of the active compound(s) for administration will depend on the judgement of the practitioner. In determining the effective amount of the active compound(s) to be administered in the production of analgesia, the physician may evaluate severity of the pain symptoms associated with nociceptive or inflammatory pain conditions and in the amount of active compound, may consider whether the patient is opioid analgesic naive or whether previous long term exposure to an opioid analgesic has occurred. In any event, those of skill in the art may readily determine suitable dosages of the derivatives of the present invention without undue experimentation.

In some embodiments, and dependent on the intended mode of administration, the pharmaceutically active derivative-containing compositions will generally contain about 0.0001% to 90%, about 0.001% to 50%, or about 0.01% to about 25%, by weight of derivative, the remainder being suitable pharmaceutical carriers or diluents etc. Illustrative unit dosages may be between about 0.01 to about 100 mg. For example, a unit dose may be from between about 0.2 mg to about 50 mg. Such a unit dose may be administered more than once a day, e.g. two or three times a day.

Depending on the specific pain condition being treated, the active compounds may be formulated and administered systemically, topically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Sublingual administration represents an example of a mode of administration. For injection, the therapeutic agents of the invention may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Alternatively, the compositions of the invention can be formulated for local or topical administration. In this instance, the subject compositions may be formulated in any suitable manner, including, but not limited to, creams, gels, oils, ointments, solutions and suppositories. Such topical compositions may include a penetration enhancer such as benzalkonium chloride, digitonin, dihydrocytochalasin B, capric acid, increasing pH from 7.0 to 8.0. Penetration enhancers which are directed to enhancing penetration of the active compounds through the epidermis are preferred in this regard. Alternatively, the topical compositions may include liposomes in which the active compounds of the invention are encapsulated. The derivatives of endomorphin, and of endomorphin analogs, of the present invention may comprise a lipidic group which, in turn, may disfavour solubilisation of that molecule in polar solvents such as water. In this respect, it may be preferable to incorporate the compounds of the present invention into liposomes. Preferably, the liposomes comprise an amphiphilic molecule such as phosphatidylcholine. The size of the liposomes may be controlled by determination of the appropriate ratio of amphiphilic molecule to derivatised endomorphin or derivatised endomorphin analog. Preferably the weight ratio of derivatised endomorphin or derivatised endomorphin analog to phosphatidylcholine is between 1:5 and 1:20. Even more preferably, the weight ratio of derivatised endomorphin or derivatised endomorphin analog to phosphatidylcholine is between 1:8 and 1:12.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives, and hydrophilic beeswax derivatives.

Alternatively, the pharmaceutically active derivatives of the present invention can be formulated readily using pharmaceutically acceptable carriers well known in the art into dosages suitable for oral administration, which is also preferred for the practice of the present invention. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulfate, vegetable oils, synthetic oils, polyols, alginic acid, phosphate buffered solutions, emulsifiers, isotonic saline and pyrogen-free water.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, preferably, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, eg. by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceuticals which can be used orally include push-fit capsules made of gelatine, as well as soft, sealed capsules made of gelatine and a plasticiser, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the active compounds of the present invention may also include injecting or implanting controlled releasing devices designed specifically for this purpose or other forms of implants modified to act additionally in this fashion. Controlled release of an active compound of the invention may be achieved by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be achieved by using other polymer matrices, liposomes and/or microspheres.

The pharmaceutically active derivatives of the invention may be administered over a period of hours, days, weeks, months or years depending on several factors, including the severity of the pain being treated, whether a recurrence of the condition is considered likely, etc. The administration may be constant, e.g., constant infusion over a period of hours, days, weeks, months, years, etc. Alternatively, the administration may be intermittent, e.g., active compounds may be administered once a day over a period of days, once an hour over a period of hours, or any other such schedule as deemed suitable. In the case of chronic pain, administration of the pharmaceutically active derivatives may take place over a period of years.

The compositions of the present invention may also be administered to the respiratory tract as a nasal or pulmonary inhalation aerosol or solution for a nebuliser, or as a microfine powder for insufflation, alone or in combination with an inert carrier such as lactose, or with other pharmaceutically acceptable excipients. In such a case, the particles of the formulation may advantageously have diameters of less than 50 micrometers, suitably less than 10 micrometers.

The invention will now be described with reference to the following examples which illustrate some preferred aspects of the present invention. However, it is to be understood that the particularity of the following description of the invention is not to supersede the generality of the preceding description of the invention.

### Example 1

### Preparation of Lipo-Amino Acids (Laa)

### rac-2-aminooctanoic acid (C8 Laa)

Sodium (2.5 g, 0.11 mol) was dissolved in ethanol (85 mL) under nitrogen and diethyl 2-acetamidomalonate (24.3 g, 0.11 mol) was added followed by 1-bromo hexane (24.5 g, 0.15 mol). The resulting solution was refluxed overnight under a nitrogen atmosphere. Upon cooling, the mixture was poured onto crushed ice (600 mL) and stirred. The precipitated product was collected and air dried. The crude product was refluxed overnight in a solution of HCl:DMF (9:1, 200 mL). Upon cooling, the precipitated product was collected, washed with ice water and air dried to afford the hydrochloride salt of the lipoamino acid: rac-2-aminooctanoic acid (C8-Laa): yield 17.9 g, (99%); MS [M+H]⁺ m/z: 160.43 ([M+H]⁺ of C₈H₁₇NO₂ requires 160.13).

Using analogous procedures, employing alternative bromo-alkanes, lipo-amino acids of different chain lengths may be synthesised.

### Example 2

### Enzymatic Resolution of rac-2-aminooctanoic acid

### D,L-2-chloroacetamido-octanoic acid

The hydrochloride salt of 2-amino-D,L-octanoic acid (4.0 g, 25.2 mmol), chloroacetyl chloride (4.12 mL, 37.8 mmol) and pyridine (2.03 mL, 25.2 mmol) were dissolved in dioxane (2.32 mL, 27.8 mmol) and ethyl acetate (180 mL) and the solution was refluxed for 5 hours. After cooling to room temperature, the reaction mixture was washed with 10% citric acid in water (50 mL×2). The organic solvent was dried (MgSO₄), removed in vacuo, and the resulting residue lyophilized from 20% acetonitrile in water. The white powder afforded was identified as chloroacetamido-α-2-aminooctanoic acid. Yield; 2.0 g, 33.8%, MS (m/z); 200.2 [M−Cl]⁺([M−Cl]⁺ of C₁₀H₁₈NO₃ requires 200.1287), ¹H NMR (400 MHz, CDCl₃) δ 8.65 (br, m, 1H, OH), 7.08 (d, 1H, J=8.2 Hz NH), 4.58 (q, 1H α-CH), 4.09 (s, 2H, CH₂Cl), 1.90 (m, 1H, β-CHH), 1.75 (m, 1H, (3-CHH), 1.25 (br m, 8H, CH₂), 0.85 (t, 3H, J=6.7 CH₃). ¹³C NMR (100 MHz, CDCl₃) δ 176.02 (NCOR), 166.41 (COOH), 52.46 (CH), 31.86, 31.45, 28.72, 25.00, 22.45 (5×CH₂), 13.94 (CH₃).

### Enzymatic Resolution of 2-chloroacetamido-octanoic acid

Method from: Birnbaum, S. M.; Fu, S. C. J.; Greenstein, J. P., Resolution of the racemic alpha-amino derivatives of heptylic, caprylic, nonylic, decylic, and undecylic acids. Journal of Biological Chemistry 1953, 203, (1), 333-338.

D,L-2-Chloroacetamido-octanoic acid (1.5 g, 6.3 mmol) was dissolved in distilled-water, and the pH of the solution was adjusted to 7.3 with 2N-lithium hydroxide (LiOH). Fresh acylase I (0.7 g) was added, and the solution was incubated at 37° C. for 2 hours. The first white precipitant was isolated, and the solution was left to stand under the same conditions for a further 72 hours. A second white precipitant was isolated, and both isolated precipitants were identified as 2-amino-L-octanoic acid. The remaining solution was acidified (pH 2) with 35% HCl and stirred overnight. The white precipitant was collected and identified as 2-amino-D-octanoic acid. 2-amino-L-octanoic acid: yield; 0.38 g, 75.8%, MS (m/z); 160.43 [M+H]⁺([M+H]⁺ of C₈H₁₇NO₂ requires 160.13) [α]_{D}; +21.4° in AcOH (c=1 g/mL) (Literature [α]_{D}; +23° in 6N HCl (c=1%)). 2-amino-D-octanoic acid: yield; 0.086 g, 17.0%, MS (m/z); 160.43 ([M+H]⁺ of C₈H₁₇NO₂ requires 160.13) [α]_{D}; −21.1° in AcOH (c=1 g/mL) (Literature [α]_{D}; +23.5° in 6N HCl (c=1%)).

### Example 3

### N-Boc Protection of 2-aminooctanoic acid

### rac-2-(tert-butoxycarbonylamino)octanoic acid

The HCl salt of rac-2-aminooctanoic acid (48 mmol) was suspended in 2-methyl-2-propanol:water (2:3, 250 mL) and the pH adjusted to 13 with 5 M sodium hydroxide. Di-tert-butyldicarbonate (72 mmol, 15.74 g) in 2-methyl-2-propanol (25 mL) was added to the Laa mixture and left to stir overnight. The pH of the mixture was checked periodically and maintained at 13 by addition of sodium hydroxide. After 16 hours, the mixture was diluted with water (100 mL) and the pH of the mixture was lowered to pH 3 by addition of solid citric acid (ca. 50 g). The product was extracted with ethyl acetate (5×150 mL), dried over magnesium sulphate and the solvent was removed under reduced pressure to yield a yellow oil. Re-crystallisation from hexane yielded white crystals. Yield 4.8 g, 40%, M.S. [M+H]⁺ m/z: 260 ([M+H]⁺ of C₁₃H₂₅NO₄ requires 260). Mp 64° C. Lit 65-67° C., ¹H NMR (400 MHz, CDCl₃) δ 4.95 (d, 1H, NH), 4.22 (m, 1H, α-CH), 1.83 (m, 1H, β-CHH), 1.66 (m, 1H, β-CHH), 1.43 (s, 9H, C(CH₃)₃), 1.25 (m, 8H, 5CH₂), 0.86 (t, 3H, J=6 CH₃). ¹³C NMR (125 MHz, CDCl₃) δ 77.6 (CO), 155.9 (CO), 80.4 (C), 53.9 (CH), 32.5 (CH₂), 31.9 (CH₂), 29.25 (CH₂), 29.10 (CH₃), 25.3 (CH₂), 22.5 (CH₂), 14.0 (CH₃).

### tert-butoxycarbonyl-L-aminooctanoic acid and tert-butoxycarbonyl-D-aminooctanoic acid

Optically pure 2-aminooctanoic acid (0.1 g, 0.63 mmol) was dissolved in DIPEA (0.2 mL). Boc-carbonate (0.16 g, 0.75 mmol) was dissolved in dioxane:water (15 mL, 2:1), and added to the DIPEA solution. The mixture was stirred for 12 hours, and the pH maintained at 8 to 9 with DIPEA. The solvent was evaporated in vacuo, and the pH was adjusted to 4 with 10% citric acid in water. The crude product was extracted with EtOAc (30 mL×2). The extracts were washed with saturated NaCl in water (20 mL×5) until the pH of the aqueous washes was 6. The EtOAc layer was dried (MgSO₄), and the solvent was removed in vacuo. The product was dissolved in 20% acetonitrile in water, and lyophilized to a white solid.

tert-butoxycarbonyl-L-aminooctanoic acid

Yield 0.105 g, 64.5%, M.S. [M+H]⁺ m/z: 260 ([M+H]⁺ of C₁₃H₂₅NO₄ requires 260), [α]_{D}²⁵=+18.9, (c=1,CHCl₃), TLC R_{f}=0.48 (CHCl₃:MeOH:AcOH; 90:8:2 v/v, 0.02% Ninhydrin in ethanol dip), ¹H NMR (400 MHz, CDCl₃) δ 5.06 (d, 1H, NH), 4.25 (q, 1H, αCH), 1.81 (m, 1H, β-CHH), 1.63 (m, 1H, β-CHH), 1.41 (s, 9H, C(CH₃)₃), 1.25 (m, 8H, CH₂), 0.84 (t, 3H, J=6.2 CH₃). ¹³C NMR (100 MHz, CDCl₃) 176.94 (CO), 155.66 (CO), 80.21 (C), 53.32 (CH), 32.21 (CH₂), 31.48 (CH₂), 28.76 (CH₂), 28.23 (CH₃), 25.17 (CH₂), 22.45 (CH₂), 13.96 (CH₃).

tert-butoxycarbonyl-D-aminooctanoic acid

Yield 0.08 g, 49%, M.S. [M+H]⁺ m/z: 260 ([M+H]⁺ of C₁₃H₂₅NO₄ requires 260), [α]_{D}²⁵=−15.8, (c=1,CHCl₃), TLC R_{f}=0.48 (CHCl₃:MeOH:AcOH; 90:8:2 v/v, 0.02% Ninhydrin in ethanol dip), ¹H NMR (400 MHz, CDCl₃) δ 5.06 (d, 1H, NH), 4.25 (q, 1H, αCH), 1.81 (m, 1H, (3-CHH), 1.63 (m, 1H, (3-CHH), 1.41 (s, 9H, C(CH₃)₃), 1.25 (m, 8H, CH₂), 0.84 (t, 3H, J=6.2 CH₃). ¹³C NMR (100 MHz, CDCl₃) δ 176.99 (CO), 155.56 (CO), 79.89 (C), 53.43 (CH), 32.43 (CH₂), 31.50 (CH₂), 28.79 (CH₂), 28.24 (CH₃), 25.14 (CH₂), 22.45 (CH₂), 13.96 (CH₃).

### Example 4

### N-(2,3,4,5-tetra-O-acetyl-β-D-glucopyranosyl)-succinate as a Building Block for N-terminal glycosylated peptides

N-(2,3,4,5-tetra-O-acetyl-β-D-glucopyranosyl)-succinate was synthesised from glucose pentaacetate via published procedures with the spectral data of the product being fully consistent with those of the literature reports (Kellam, B.; Drouillat, B.; Dekany, G.; Starr, M. S.; Toth, I., Synthesis and in vitro evaluation of lipoamino acid and carbohydrate modified enkephalins as potential antinociceptive agents. International Journal of Pharmaceutics 1998, 161, (1), 55-64 and Blanchfield, J. T.; Toth, I., Modification of peptides and other drugs using lipoamino acids and sugars. In Peptide Synthesis and applications, Howl, J., Ed. Humana Press: Totawa, N.J., 2005; Vol. 298, pp 45-61).

### Example 5

### 2,3,4-tri-O-acetyl-1-azido-1-deoxy-β-D-glucopyranuronic acid as a Building Block for N-terminal glycosylated peptides

2,3,4-tri-O-acetyl-1-azido-1-deoxy-β-D-glucopyranuronic acid was synthesized from glucuronic acid and immobilized onto Rink amide MBHA resin via literature procedure (Blanchfield, J. T.; Toth, I., Modification of peptides and other drugs using lipoamino acids and sugars. In Peptide Synthesis and applications, Howl, J., Ed. Humana Press: Totawa, N.J., 2005; Vol. 298, pp 45-61 and Malkinson, J. P.; Falconer, R. A.; Toth, I., Synthesis of C-terminal glycopeptides from resin-bound glycosyl azides via a modified Staudinger reaction. Journal of Organic Chemistry 2000, 65, (17), 5249-5252).

### Example 6

### Synthesis of N-(tert-butoxycarbonyl)-2′,6′-dimethyl-L-tyrosine (DMT)

N-(tert-butoxycarbonyl)-2′,6′-dimethyl-L-tyrosine was prepared as shown in Scheme 1 according to reported methods (Dygos, J. H.; Yonan, E. E.; Scaros, M. G.; Goodmonson, O. J.; Getman, D. P.; Periana, R. A.; Beck, G. R., A convenient asymmetric synthesis of the unnatural amino-acid 2,6-dimethyl-L-tyrosine. Synthesis-Stuttgart 1992, (8), 741-743).

### Example 7

### Preparation and Reaction of Amino Aldehydes

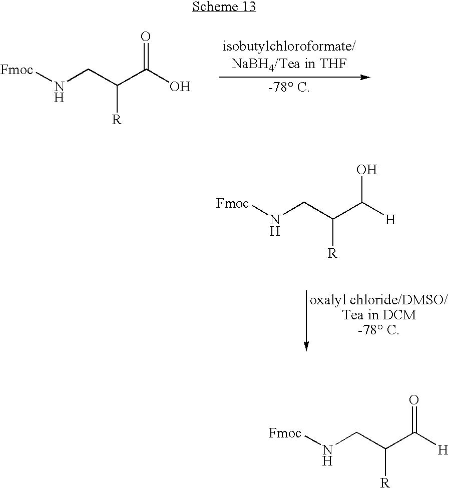

An Fmoc protected amino acid was reduced to the alcohol by reaction with isobutylchloroformate/NaBH₄/TEA in THF at −78° C. (Scheme 13). The resulting alcohol was then oxidised to the corresponding aldehyde by reaction with oxalyl chloride/DMSO/TEA in DCM at −78° C.

Analytical data for R═C₈H₁₇ (Scheme 13): Yield=49%; R_{f}=0.33 (1:1 hexane/ethylacetate); [M+H]⁺=245.4.

Analytical data for R=benzyl (Scheme 13): Yield=55%; R_{f}=0.35 (1:1 hexane/ethylacetate); [M+H]⁺=388.2.

The amino aldehydes were coupled to a growing peptide chain, bound to a solid support, by reaction of a 2-fold excess of the amino aledehyde in the presence of 2.1 equivalents of DIPEA in DMF. Coupling was determined by ninhydrin assay at 60 minutes.

### Example 8

### General Method for Solid Phase Peptide Synthesis

Peptides were assembled on Rink amide MBHA resin (100-200 mesh, 0.78 mmol/g loading) on a 0.5 mmol scale using HBTU/DIPEA activation and the in situ neutralisation protocol (Alewood, P.; Alewood, D.; Miranda, L.; Love, S.; Meutermans, W.; Wilson, D., Rapid in situ neutralization protocols for Boc and Fmoc solid-phase chemistries. Solid-Phase Peptide Synthesis 1997, 289, 14-29). The following protected amino acids were used: Fmoc-Phe, Fmoc-Trp(Boc), Fmoc-Pro, Fmoc-Tyr(tBu). The efficiency of each amino acid coupling was determined by the quantitative ninhydrin reaction (Robyt, J. F.; White, B. J., Biochemical techniques theory and practice. WAVELAND PRESS, INC.: 1987) and couplings repeated until an efficiency >99.6% was achieved. The chloranil test (Vojkovsky, T., Detection of secondary-amines on solid phase. Peptide Research 1995, 8, (4), 236-237 and Chan, W. C.; White, P. D., Fmoc solid phase peptide synthesis: a practical approach. Oxford University Press, Oxford: 2000) was performed to determine the coupling efficiency for amino acid coupled to a proline residue.

The N-Dde protected Laas were coupled to the growing peptides using standard coupling techniques, with the reaction allowed to proceed for 1 hour and repeated once. The, N-Dde protecting group was removed by treatment with 2% hydrazine hydrate in DMF (1 hour×2) followed by efficient rinsing of the resin with DMF as usual. The removal of O-acetyl groups of glucose succinamide and glucuronic acid was carried out after removal of N-terminal protecting groups. The drained resin-peptide was suspended in 5 mL of 12.5% (v/v) hydrazine hydrate in methanol and the suspension mixed for 18 hours at room temperature. The resin was then drained and washed well with DMF before preparing for cleavage as usual.

When construction of the peptide was complete the resin was washed with DMF, DCM and MeOH and dried. The peptide was cleaved from the resin by treatment with TFA:water:triisopropylsilane (TIS) (95:2.5:2.5 v/v, 25 mL) for 6 hours. The resin was removed by filtration and washed with TFA. The solvent was removed from the peptide solution under a stream of nitrogen and the crude peptide precipitated with cold diethyl ether, collected and dissolved in 20% acetonitrile and lyophilized.

The purification of peptide analogs was achieved by preparative RP-HPLC on a Waters; 600 controller and pump with a 490E programmable multi wavelength detector. The purity of peptide analogs was determined by analytical RP-HPLC (Shimazu; SCL-10AVP system controller, FCV-10ALVP pump, and SPD-6A UV detector), electrospray ionization MS (ESI-MS; Perkin-Elmer Sciex API 3000) and LC/MS (Shimadzu LC-10AT HPLC, Perkin-Elmer Sciex API 3000).

### Example 9

### General Method for Solution Phase Peptide Synthesis

Peptides were also synthesized by a segment condensation method (Jinsmaa, Y.; Marczak, E.; Fujita, Y.; Shiotani, K.; Miyazaki, A.; Li, T. Y.; Tsuda, Y.; Ambo, A.; Sasaki, A.; Bryant, S. D.; Okada, Y.; Lazarus, L. H., Potent in vivo antinociception and opioid receptor preference of the novel analogue [Dmt¹]endomorphin-1. Pharmacology, Biochemistry and Behavior 2006, 84, 252-258 and Li, T. Y.; Fujita, Y.; Tsuda, Y.; Miyazaki, A.; Ambo, A.; Sasaki, Y.; Jinsmaa, Y.; Bryant, S. D.; Lazarus, L. H.; Okada, Y., Development of potent μ-opioid receptor ligands using unique tyrosine analogues of endomorphin-2. Journal of Medicinal Chemistry 2005, 48, (2), 586-592).

### Example 10

### Permeability Study

### Cell Culture

Caco-2 cells were maintained in Dulbecco's modified Eagle's medium (DMEM) supplemented with 10% foetal bovine serum (FBS), 1% L-glutamine and 1% nonessential amino acids at 95% humidity and 37° C. in the atmosphere of 5% CO₂. The medium was changed every other day and the cells were subcultured using 0.25% trypsin when they reached 80% confluence.

### Permeability Study

Approximately 1×10⁶ cells/mL concentration of cells were seeded onto polycarbonate cell culture inserts (Transwell®; 0.45 μm×6.5 mm diameter). DMEM cell culture media was supplemented with 10% FBS, 1% L-glutamine, 1% nonessential amino acids and 1% 100 U/mL penicillin/streptomycin. The media was changed every other day (0.6 mL in the basolateral chamber and 0.1 mL to the apical chamber) and the monolayers were used between days 21-28. Transepithelial electrical resistance (TEER) of the monolayer was measured using the Millicell-ERS system (Millipore Corporation, Bedford, Mass.). C¹⁴-mannitol permeability was used to monitor the integrity of the Caco-2 monolayers.

Transport studies were carried out in Hanks' balanced salt solution (HBSS) with 25 mM Hepes (pH 7.4) at 37° C. Compounds to be tested were dissolved in HBSS-Hepes buffer to a final concentration 200 μM. If compounds were not water soluble to this extent, DMSO was added to aid solubilisation to a concentration not more than 4% in the final solution (usually 0.5% DMSO was adequate). Prior to beginning the experiments, the monolayers were washed with pre-warmed HBSS-Hepes buffer, incubated for 30 min. At the start of the experiment the apical chamber was emptied and 100 μL of either the derivatised endomorphin or derivatised endomorphin analog solution was added. The permeability study was performed in triplicate at 37° C. shaking in a Heridolf Titramax shaking at 400 rpm. Samples (400 μL) were taken at the regular interval time points (30, 90, 120, 150 min) and replaced with the same volume of buffer. Permeability coefficients (Pₐₚₚ) were calculated from data generated by LC/MS quantitative analysis of all samples using a gradient HPLC system (Shimadzu LC-10AT system) coupled to a triple quadrupole mass spectrometer (PE Sciex API 3000) operating in SIM mode with positive ion electrospray. The mobile phase was a mixture of solvent A (0.1% formic acid in water) and solvent B (0.1% formic acid in 90% acetonitrile/water). A C18 column, 5 μM, 50×2.0 mm (Phenomex®) was used at a flow rate 0.3 mL/min with a gradient from 100% A to 90% B in 7 minutes, incorporating a splitter (1:10).

Pₐₚₚ=dC/dt×Vᵣ/(A×C₀)

dC/dt=steady−state rate of change in the chemical concentration (mol s⁻¹) or radiochemical concentration (dpm mL⁻¹s⁻¹) in the receiver chamber,

Vᵣ=volume of the receiver chamber (mL),

A=surface area of the cell monolayers; and

C₀=initial concentration in the donor chamber (mol or dpm mL⁻¹).

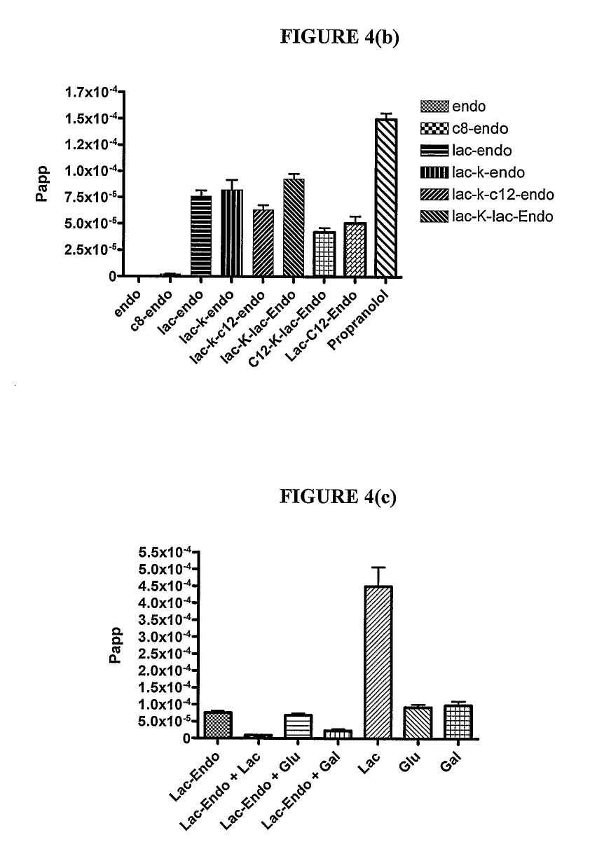

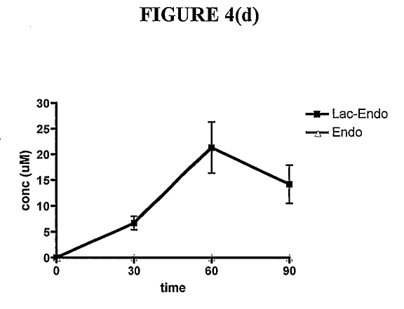

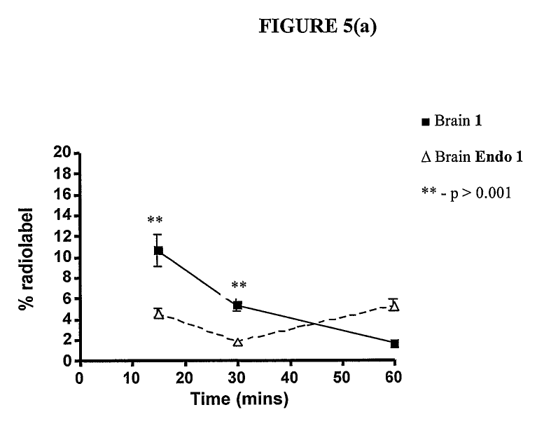

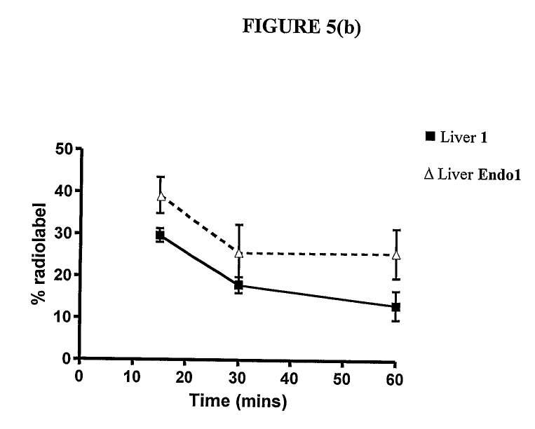

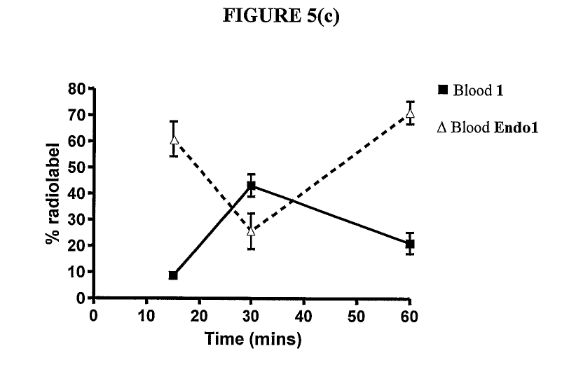

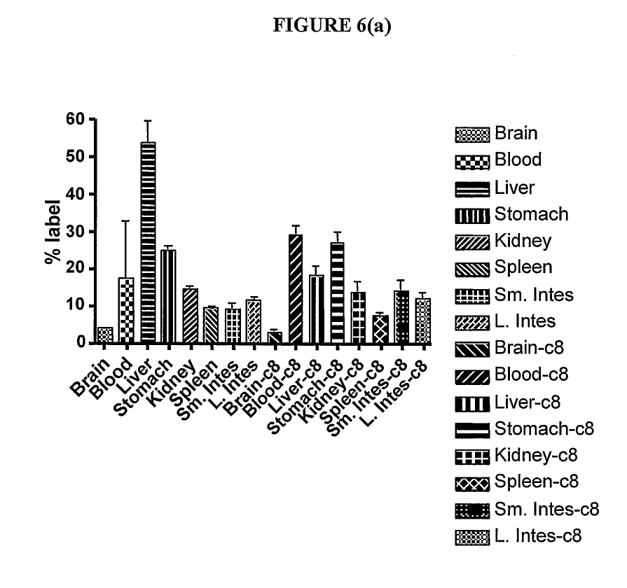

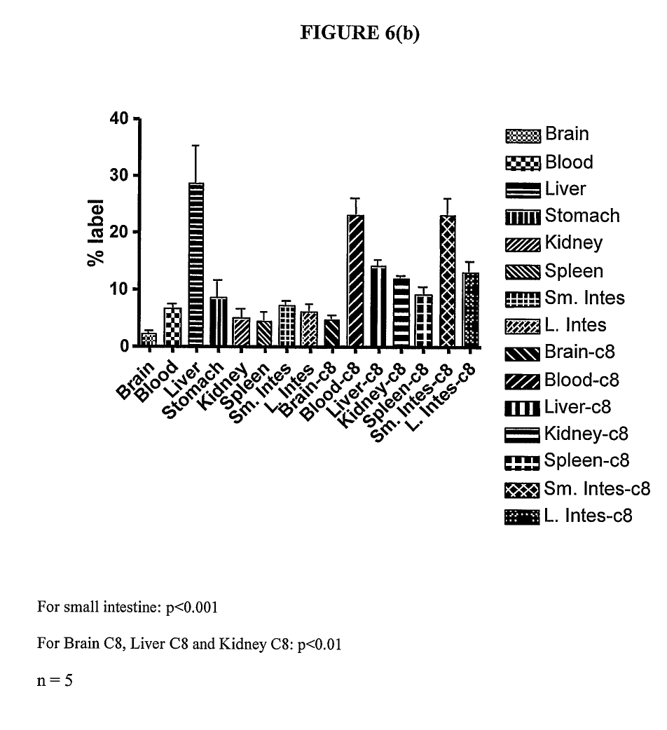

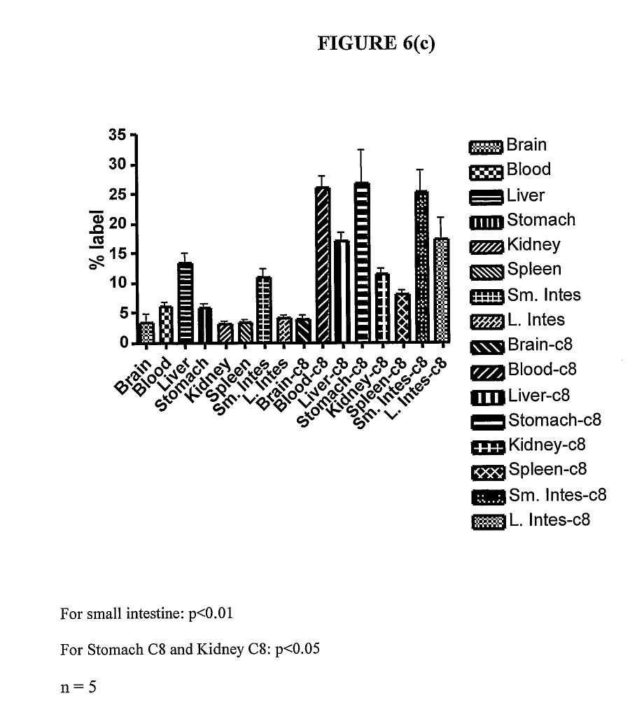

See FIG. 4(a).

In a further experiment, it was interestingly discovered that incorporation of saccharide substituents (such as lactose) onto the endomorphin structure dramatically increased the permeability of the peptide across Caco-2 cell monolayers. See FIG. 4(b). All of the lactose conjugates exhibited excellent permeability across Caco-2 cell monolayers and were orders of magnitude more permeable than the parent compound, Endo-1 or the lipid appended compound, 1 from Table 2. Propranolol was used as a positive control.

In order to test whether this effect was due to lactose recognition by a receptor on the cell surface which binds the sugar and then promotes pinocytosis or by a lactose selective transporter, the Caco-2 cell permeability assay for Lac-endo was repeated but this time in the presence of 20 mM lactose, glucose or galactose. The permeability of the sugars themselves was also examined. See FIG. 4(c). It was clear that lactose severely inhibits the absorption of Lac-endo suggesting that this effect involves selective binding of lactose. Galactose, the terminal monosaccharide of lactose also causes some inhibition of absorption but less than lactose and glucose doesn't inhibit the absorption of Lac-endo. These results suggest that the absorption is not occurring through any of the known glucose transporters in the Caco-2 cell monolayer.

In another experiment, Lac-endo was fed via oral gavage to male rats and the concentration of the compound in blood plasma measured over time. Sprague-dawley rats (average wt 300 g) were gavaged with 500 μL of a 20 mM Lac-endo or Endomorphin solution in water. Blood samples taken from the tail vein after 30, 60 and 90 min. The blood was centrifuged to remove red blood cells, and in a 100 μL sample of plasma was added to 300 μL of acetonitrile to precipitate proteins. The suspension was centrifuged a second time and the supernatant analysed by LC-MS. See FIG. 4(d). The presence of the lactose moiety resulted in significant absorption of the compound across the GI tract while the native peptide is not absorbed.

In summary, it has been shown that derivatisation of an endomorphin, or an endomorphin analog, with a saccharide moiety (for example a lactose moiety) enhances GI uptake, and thus provides a handle to enhance uptake associated with oral delivery.

### Example 11

### Stability Study

### Enzymatic Stability Study in Homogenized Cells

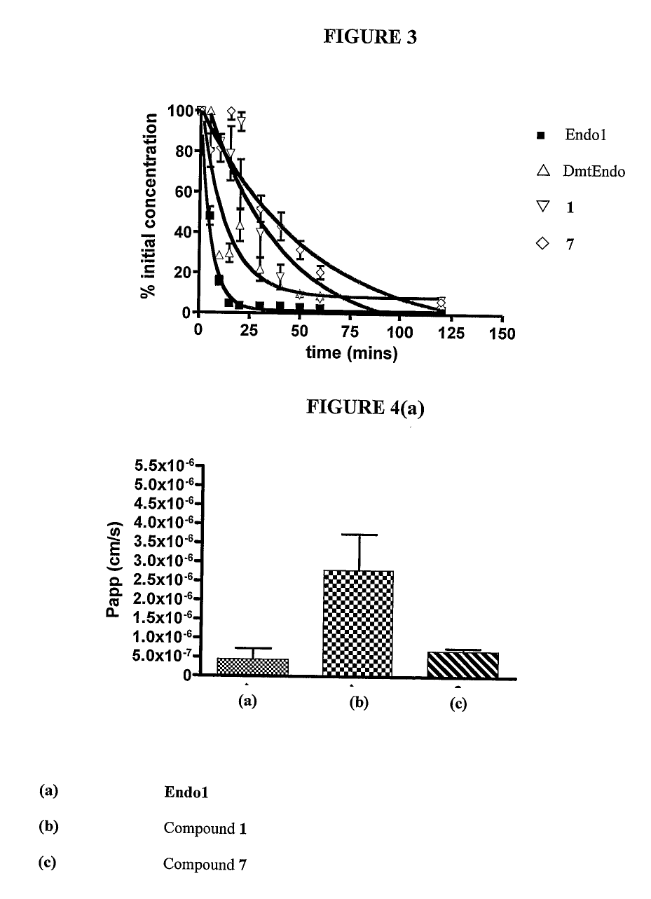

Approximately 1×10⁵ cells/mL concentration of cells were seeded onto the 96 well plate. DMEM cell culture media was supplemented with 10% FBS, 1% L-glutamine, 1% nonessential amino acids and 1% of 100 U/mL penicillin/streptomycin. The media was changed every other day. 21-28 days old cells were used for the experiments. the media was removed from the wells and each well washed with 100 μL 0.2% EDTA solution followed by washing three times with Hanks' balanced salt solution (HBSS) containing 25 mM Hepes (pH 7.4). Finally, 100 μL of this buffer was placed in each well and the plate cooled in ice. The monolayer of cells in each well was then disrupted by 2×1 second pulses with a Sonics Vibracell ultrasonic processor set at an amplitude of 30. The cell debris was then removed by centrifugation of the plate at 2000 rpm for 5 min. 80 μL cell homogenate supernatant of each well was then transferred to a clean 96 well plate, and 20 μL of HBSS-Hepes was added. Three of the wells' contents were set aside and assayed for total protein content using the Lowry assay. The compounds to be tested were dissolved in HBSS-Hepes buffer to a concentration of 200 μM. To begin the assay, 100 μL of either the derivatised endomorphin or derivatised endomorphin analog solution was added to each well containing cell homogenate so the final concentration of test compound at the beginning of the experiment was 100 μM. Samples (10 μL) were taken at selected time points (1, 5, 10, 15, 20, 30, 40, 50, 60, 120 min.) and immediately added to 5 μl, of TFA to stop digestion and then diluted with 80 μL of water (see FIG. 3).

Compound half-life (mins)

| | |
|---|---|
| Endo | 3.914 |
| Dmt-Endo | 8.983 |
| Compound 1 | 25.54 |
| Compound 7 | 37.40 |

This assay was performed at 37° C. The concentration of the derivatised endomorphin or derivatised endomorphin analog in each sample was determined by LC-MS using a gradient HPLC system (Shimadzu LC-10AT system) coupled to a triple quadrupole mass spectrometer (PE Sciex API 3000) operating in SIM mode with positive ion electrospray. The mobile phase was a mixture of solvent A (0.1% formic acid in water) and solvent B (0.1% formic acid in 90% acetonitrile/water). A C18 column, 5 μM, 50×2.0 mm (Phenomex®) was used for RP-HPLC. Flow rate 0.3 ml/min with a gradient from 100% A to 90% B in 7 minutes, incorporating a splitter (1:10). The stability assay on each compound was conducted in triplicate and the concentrations averaged for each time point.

### Example 12

### Cell Based Assay of Endomorphin-1-Induced cAMP Inhibition

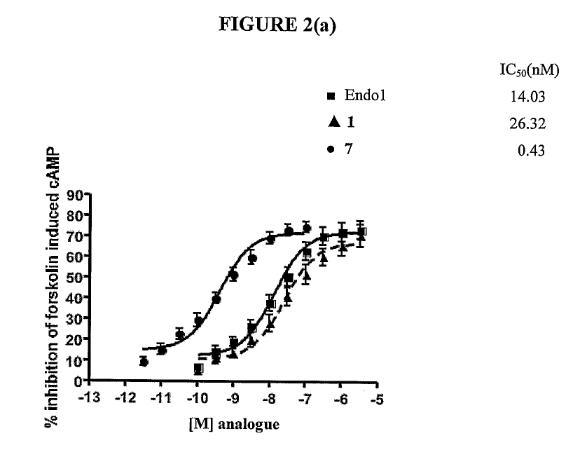

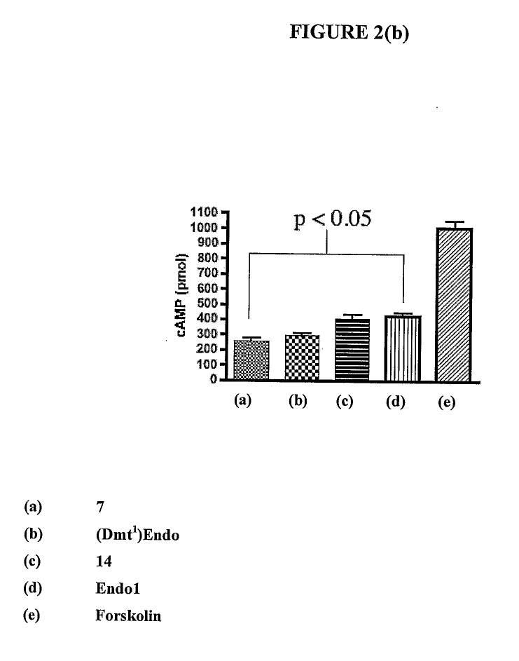

SH-SY5Y cells endogenously expressing μ- and δ-opioid receptors were cultured (100 μl) in a 96 well-plate and incubated (usually overnight) until 80% confluent. Once cells were ready for assay, Endo-1, Endo-2 and derivatives/analogs were diluted in DMEM culture medium containing 30 μM forskolin. The diluted derivatised endomorphins or derivatised endomorphin analogs were then applied to separate wells for agonist studies and co-incubated for antagonist studies. Cells were incubated with derivatised endomorphins or derivatised endomorphin analogs at 37° C. for 30 minutes, medium was aspirated and cells were lysed using lysis buffer from a cAMP Biotrak EIA kit (Amersham, Buckinghamshire, UK). An anti-cAMP antibody was added to the lysate, and the mixture incubated at 4° C. for 2 hours followed by the addition of a cAMP peroxidase conjugate, and incubated at 4° C. for a further hour. A 3,3′5,5′-tetramethylbenzidine (TMB) substrate provided by the Biotrak EIA kit was then incubated with cell lysate for 1 hour and finally 1 μM sulphuric acid was added to facilitate a colour change, which is proportional to the amount of cAMP captured. A cAMP standard curve from 0 to 3200 fmol was also generated concurrently. Plates were then read by a plate reader at 450 nm and compared back to the cAMP standard curve. All data are compared to the maximum cAMP response to forskolin and are represented as the mean+/−standard error of three experiments performed in triplicate. All data are plotted using PRISM program (GraphPad, San Diego, Calif.) (see FIGS. 2a and 2b).

### Example 13

### Whole Cell-Based Binding Assays

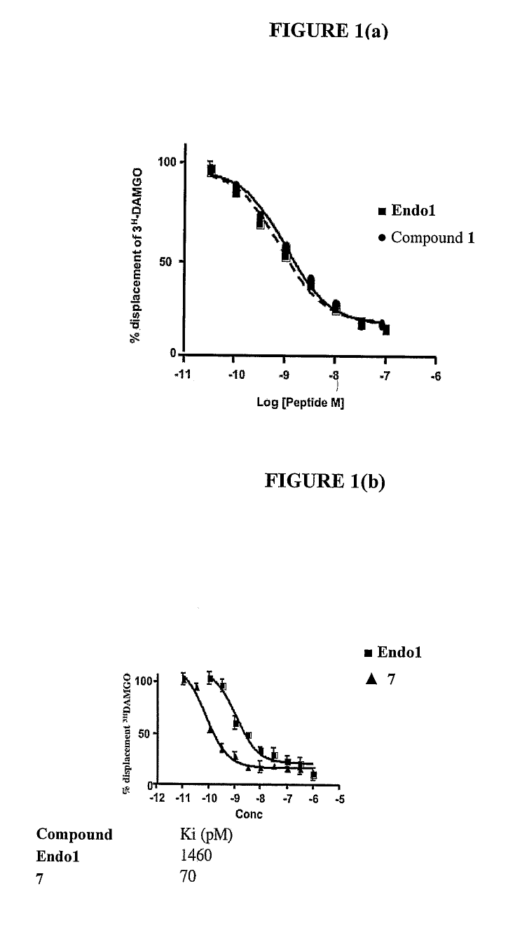

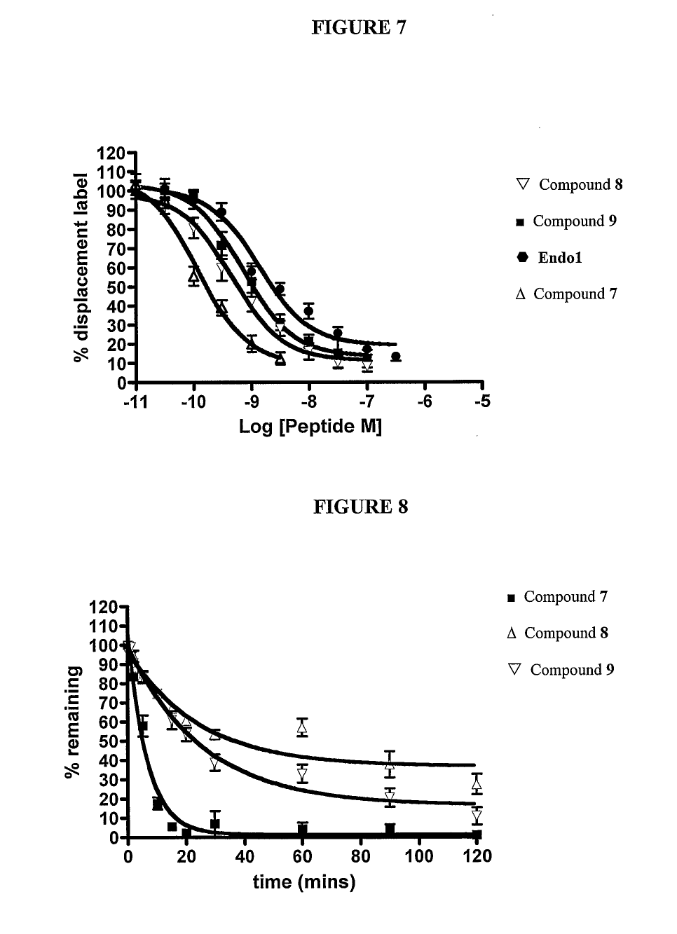

To measure total binding of Endo-1 and synthesised analogs, SH-SY5Y cells were plated with binding buffer (50 mM Tris buffer, pH 7.4, 2% BSA) in 24 well plates and incubated overnight. Media was removed and cells were washed with PBS before pre-incubation in 300 μL of binding buffer. Binding studies were performed with 100 μL of appropriate concentrations of ³H-labeled DAMGO (a selective μ-opioid receptor agonist) and 100 μL of competitor or blank in binding buffer at 37° C. for 60 min. Competition binding experiments were performed using 100 μM of ³H-labelled DAMGO in the absence or presence of increasing concentrations of unlabeled derivatised endomorphins or derivatised endomorphin analogs. A 1 μM excess of unlabelled DAMGO was used to determine non-specific binding. Following incubation, the cells were washed with PBS and recovered from culture plates using 500 μL of 1 μM NaOH before transfer to scintillation vials. Liquid scintillation cocktail (Ultima Gold, Packard, Meriden, USA) was added to these vials for counting in a liquid scintillation analyser. Data are expressed as the mean±SEM of % specific binding of triplicate determinations performed on at least three independent plates of cells. Data were plotted using the one-site competition functions of the PRISM program (Graphpad Inc., San Diego, USA) (see FIGS. 1a, 1b and 7).

### Example 14

### Plasma Stability Assay

To measure the enzymatic stability of endomorphin-1 analogs in plasma, blood was taken from a healthy male volunteer and the blood was centrifuged at 1700 rpm for 30 minutes to remove blood cells. The plasma was placed into a water bath heated at 37° C. and Endomorphin-1 and analog was added to give final concentrations of 0.7 mg/mL. Samples were incubated for varying time periods between 0 and 180 minutes. 50 μL of sample was taken at each time-point and 75 μL of acetonitrile was added to quench any enzyme activity. The samples were then placed on ice and upon completion of the assay, all samples were centrifuged at 9000 rpm for 3 minutes to remove all precipitated plasma proteins. The amount of intact peptide remaining in the supernatent of each sample was determined using RP HPLC by measuring the area under the curve of the peak at the appropriate retention time compared to a standard curve of concentration for each peptide (see FIG. 8).

The results for each analog are displayed in Tables 3 and 4.

TABLE 3
Summary of binding and cAMP functional assay data.

| | EfficacySH-SY5Y cell line | | | |
|---|---|---|---|---|
| | Binding | | Inhibit c-AMP | Plasma Stability |
| Compound | (Kᵢ) (nM) | IC₅₀ (nm) | (%)* | (mins) |
| (Dmt¹)Endo | 0.058 | 0.156 | 160 | 4.18 |
| Endo1 | 1.45 | 19.5 | 100 | 3.45 |
| 1 | 2.45 | | 97 | |
| 2 | 35.5 | | 89 | |
| 4 | 115.0 | | 52 | |
| 5 | 148 | | 52 | |
| 6 | 4.27 | | 98 | |
| 7 | 0.071 | 0.189 | 153 | 4.55 |
| 8 | 0.47 | 0.0634 | | 41.35 |
| 9 | 0.735 | 0.928 | | 20.29 |
| 10 | 42.4 | | 94 | |
| 12 | N.D. | | 90 | |
| 13 | 265 | | 42 | |
| 14 | 5.19 | | 96 | |
| 16 | 108.7 | | 59 | |
| 17 | 163.8 | | 59 | |
| 18 | 151.2 | | 47 | |
| 19 | 121.9 | | 51 | |

*normalised to the % of cAMP inhibition seen by native endomorphin-1

TABLE 4
Table of binding and stability data

| | Drug delivery | | | | | | | | | | Efficacy | | | | | | | | | |
|---|---|---|---|---|---|---|---|---|---|---|---|---|---|---|---|---|---|---|---|---|
| | Caco-2 cell line | | | | | | | | | | SH-SY5Y cell line | | | | | | | | | |
| | Enzymatic stability | | | | | Permeability | | | | | Binding | | | | | | | | | |
| Compound | 2 h | | | | | (cm/s) | | | | | μ-opioid receptor | | | | | Inhibit c-AMP | | | | |
| Endo1 | ♦ | | | | | ♦ | | | | | ♦ | ♦ | ♦ | ♦ | | ♦ | ♦ | ♦ | ♦ | |
| (Dmt¹)Endo | ♦ | | | | | ♦ | * | | | | ♦ | ♦ | ♦ | ♦ | ♦ | ♦ | ♦ | ♦ | ♦ | ♦ |
| 1 | ♦ | ♦ | ♦ | ♦ | | ♦ | ♦ | ♦ | ♦ | | ♦ | ♦ | ♦ | ♦ | | ♦ | ♦ | ♦ | | |
| 2 | ♦ | ♦ | ♦ | ♦ | | ♦ | ♦ | ♦ | | | ♦ | ♦ | ♦ | | | | | | | |
| 4 | ♦ | ♦ | ♦ | ♦ | | ♦ | ♦ | | | | ♦ | ♦ | | | | ♦ | ♦ | | | |
| 5 | ♦ | ♦ | ♦ | ♦ | | ♦ | ♦ | ♦ | ♦ | | ♦ | ♦ | ♦ | | | ♦ | ♦ | | | |
| 6 | | | | | | ♦ | ♦ | ♦ | ♦ | | ♦ | ♦ | ♦ | ♦ | | ♦ | ♦ | ♦ | ♦ | |
| 7 | ♦ | ♦ | ♦ | ♦ | | ♦ | ♦ | ♦ | * | | ♦ | ♦ | ♦ | ♦ | ♦ | ♦ | ♦ | ♦ | ♦ | ♦ |
| 10 | | | | | | ♦ | ♦ | | | | ♦ | ♦ | | | | ♦ | ♦ | | | |
| 12 | ♦ | ♦ | ♦ | ♦ | ♦ | ♦ | ♦ | ♦ | ♦ | ♦ | ♦ | ♦ | | | | ♦ | ♦ | ♦ | | |
| 13 | ♦ | ♦ | ♦ | ♦ | | | | | | | ♦ | | | | | ♦ | | | | |
| 14 | | | | | | | | | | | ♦ | ♦ | ♦ | ♦ | | ♦ | ♦ | ♦ | ♦ | |
| 16 | | | | | | ♦ | ♦ | ♦ | | | ♦ | ♦ | | | | ♦ | | | | |
| 17 | | | | | | ♦ | ♦ | | | | ♦ | | | | | ♦ | | | | |
| 18 | ♦ | | | | | ♦ | | | | | ♦ | ♦ | ♦ | | | ♦ | ♦ | | | |
| 19 | ♦ | | | | | ♦ | ♦ | | | | ♦ | ♦ | | | | ♦ | ♦ | | | |

### Example 15

### Pain Relief in Rodent Model

An established rodent model of neuropathic pain (rats with a chronic constriction injury (CCI) of the sciatic nerve) was used to investigate the anti-allodynic (ipsilateral hindpaw) and antinociceptive (contralateral hindpaw) efficacy and potency of single intravenous and sun-cutaneous (s.c.) bolus doses of Compound 8 relative to that of the parent peptide Endomorphin 1 (Endo1) and vehicle.

General Methods

A 90%:10% mixture of DMSO:water was used as the vehicle for this study. Each dose of Compound 8, Endomorphin-1 (Endo1) and vehicle was administered to CCI-rats as a 100 μL intravenous (i.v.) bolus injection via an indwelling cannula surgically implanted into the jugular vein at least 24 h prior to dose administration, or as a 100 μL sub-cutaneous (s.c.) bolus injection.

Chronic Constriction Injury (CCI) of the sciatic nerve was produced by tying four loose ligatures around the left sciatic nerve according to the method of Bennett and Xie (1988). This is a rat model of peripheral mononeuropathy which results in the development of tactile allodynia (exaggerated response to the application of a non-noxious stimulus such as light pressure or touch). The ability of single bolus i.v. doses of Endo1 Compound 8 or vehicle to alleviate tactile allodynia (defining symptom of neuropathic pain) was assessed using calibrated von Frey filaments. The contralateral (non-injured) hindpaw of the same animal served as an internal control.

Commencing with the von Frey filament that produced the lowest force, the filament was applied to the plantar surface of the hindpaw until the filament buckled slightly. Absence of a response after 5 s prompted use of the next filament of increasing weight. Filaments used produced a buckling weight of 2, 4, 6, 8, 10, 12, 14, 16, 18 and 20 g. A score of 20 g was given to animals that did not respond to any of the von Frey filaments. Assessment of paw withdrawal thresholds using von Frey filaments was undertaken prior to CCI-surgery and on day 14 post-CCI surgery. Additionally, von Frey paw withdrawal thresholds (PWTs) were quantified pre-dose and at the following post-dosing times after administration of each i.v. dose of Endo1, Compound 8 or vehicle: 0.25, 0.5, 0.75, 1, 1.25, 1.5, 2, 3 h.

Single bolus i.v. doses of vehicle (n=3), Endo1 at 1 mg/kg (n=5) or Compound 8 at 1 mg/kg (n=8) were administered to opioid-naïve CCI-rats and paw withdrawal thresholds were assessed. Mean(±SEM) PWT versus time curves were plotted for each route of administration for Endo1, Compound 8 and vehicle in CCI-rats. Single bolus s.c. doses of vehicle or Compound 8 at 0.1 (n=4), 0.3 (n=6), 1 (n=4), 3 (n=6), and 10 (n=3) mg/kg were administered to opioid-naive CCI-rats and paw withdrawal thresholds were assessed. Mean(±SEM) PWT versus time curves were plotted for each route of administration for Compound 8 and vehicle in CCI-rats. PWT values were also normalized by subtracting the respective pre-dosing baseline values and the areas under the normalized response versus time curves (AUC values) were estimated using trapezoidal integration. Dose-normalised AUCs were calculated by dividing AUC by the dose of the test article in μmol/kg.

The Mann-Whitney or Kruskall Wallis nonparametric tests, as implemented in the GraphPad Prism™ statistical analysis program (v3.0) were used to compare (i) von Frey paw withdrawal thresholds before and after CCI-surgery (ii) the effect of Endo1, Compound 8 or vehicle on von Frey paw withdrawal thresholds after CCI-surgery (iii) the effect of Endo1, Compound 8 or vehicle on dose-normalised AUC.

Results for i.v. Injection

Tactile (mechanical) allodynia developed in the ipsilateral but not the contralateral hindpaw of rats following the induction of a unilateral chronic constriction injury (CCI) of the sciatic nerve. Specifically, the mean(±SEM) von Frey paw withdrawal thresholds (PWTs) for the ipsilateral hindpaw decreased significantly (p<0.05) from 11.1 (±0.2) g to 4.2 (±0.2) g by 14 days post-CCI surgery. The mean(±SEM) PWT value for the contralateral hindpaw did not differ significantly (p>0.05) between that determined prior to CCI-surgery (11.1±0.2 g) and that determined 14 days later in the contralateral hindpaw (10.9±0.1 g) of the same animals.

Experiment 1: The Anti-Allodynic Effect of i.v. Endo1 and Compound 8 in CCI-Rats

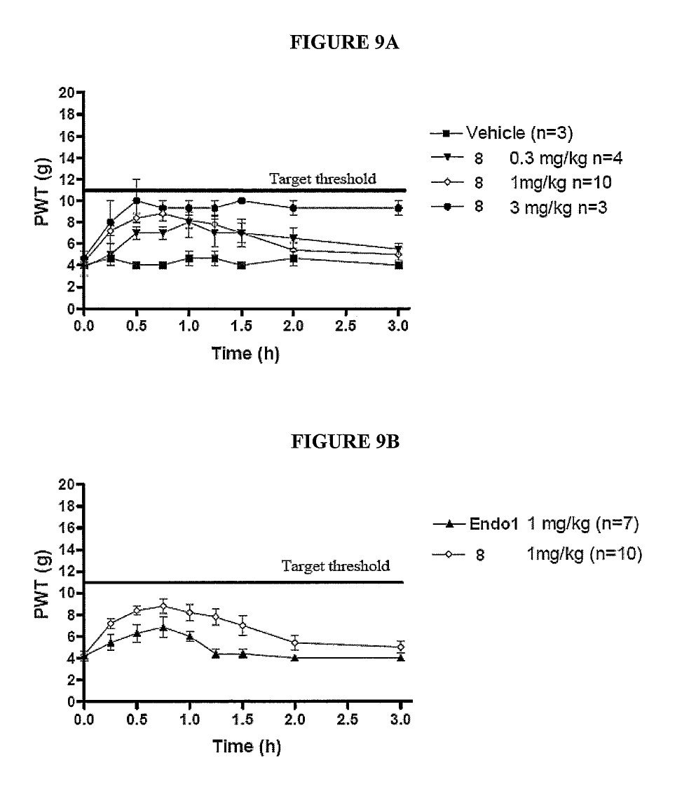

Single bolus i.v. doses of Compound 8 produced a significant, dose-dependent anti-allodynic effect in the ipsilateral hindpaw, compared with that of vehicle (FIG. 9A). Specifically, mean peak anti-allodynic responses were observed at ˜0.5-1 h post-dosing. At the highest dose tested (3 mg/kg), the mean duration of action was >3 h. At the lower doses (0.1 and 0.3 mg/kg), the mean duration of action was ˜2-3 h. Following i.v. administration of single bolus doses of the parent peptide, Endo1 (1 mg/kg) to adult male CCI-rats, a brief period of significant anti-allodynia was observed with the mean peak response observed at 0.75 h post-dosing and a corresponding duration of action of ˜1.25 h (FIG. 9B).

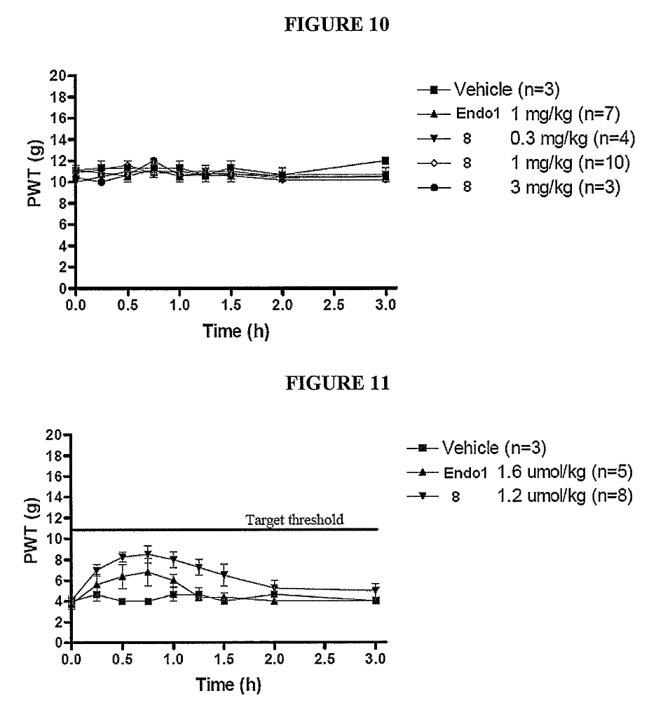

In the contralateral hindpaw, i.v. bolus doses of vehicle, Endo1 (1 mg/kg) and Compound 8 (0.3 mg/kg-3 mg/kg) did not significantly alter baseline PWT responses for the dose range investigated (FIG. 10).

Experiment 2: Comparison of the Anti-Allodynic Efficacy and Potency of i.v. Compound 8 and Endo1 in Drug-Naïve CCI-Rats

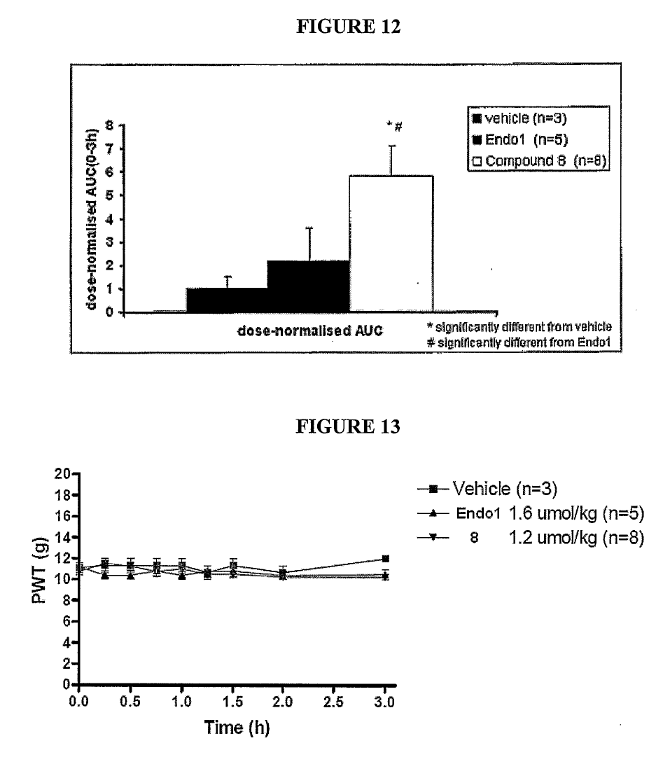

As Compound 8 has a larger molecular weight at 821 g/mol compared with Endo1 at 611 g/mol, the doses were converted to μmol/kg. Hence, the 1 mg/kg dose of Endo1 equates to 1.6 μmol/kg and for Compound 8 the 1 mg/kg dose is 1.2 μmol/kg. Following i.v. administration of single bolus doses of the parent peptide, Endo1 at 1.6 mmol/kg to drug-naïve adult male CCI-rats, a brief period (˜1 h) of significant anti-allodynia was observed (FIG. 11). By contrast, a single bolus i.v. dose of Compound 8 at 1.2 mmol/kg produced a significantly (p<0.05) larger extent and duration of anti-allodynia in the ipsilateral hindpaw, compared with that of Endo1 (1.6 mmol/kg) (FIG. 11). FIG. 12 shows that the dose-normalised area under the anti-allodynic response versus time curve (AUC) in the ipsilateral hindpaw for Compound 8 is significantly greater (p<0.05) than that of both Endo1 and vehicle. By contrast, the corresponding dose-normalised AUC for i.v. Endo1 was not significantly different (p>0.05) from that of vehicle.

For the contralateral hindpaw of drug-naïve CCI-rats, i.v. bolus doses of vehicle, Endo1 (1.6 μmol/kg) and Compound 8 (1.2 μmol/kg) did not significantly alter baseline PWT responses (FIG. 13).

Results for s.c. injection

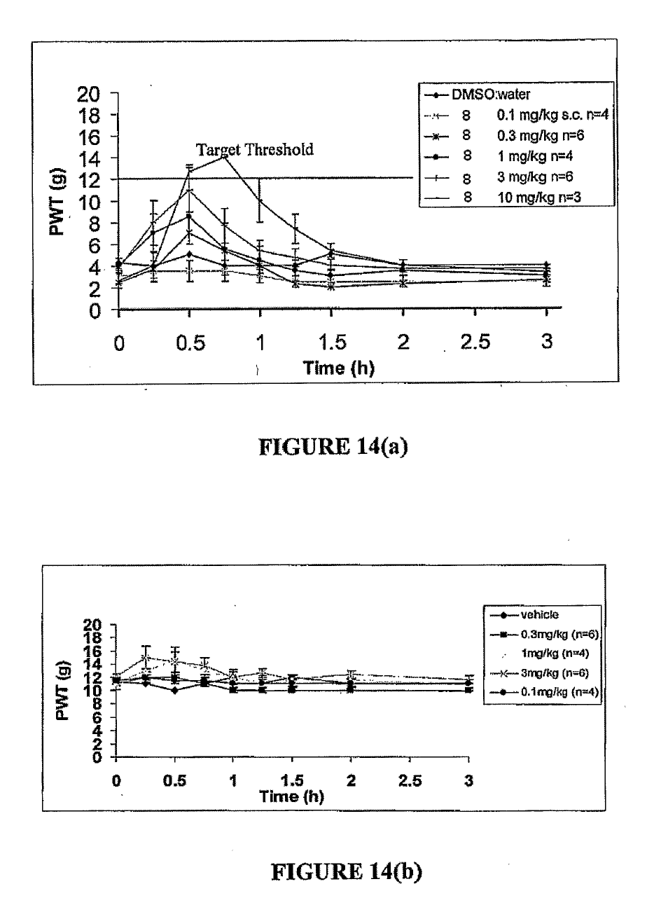

Single bolus s.c. doses (0.1-10 mg/kg) of Compound 8 produced a significant, dose-dependent anti-allodynic effect in the ipsilateral hindpaw, compared with that of vehicle (FIG. 14(a)). Specifically, mean peak anti-allodynic responses were observed at ˜0.5-1 h post-dosing. Single bolus s.c. doses (0.3-3 mg/kg) of Compound 8 did not produce a significant, dose-dependent anti-nociceptive effect in the contralateral hindpaw, compared with that of vehicle (FIG. 14(b)).

### SUMMARY

Single bolus i.v. doses of modified Endo1, namely Compound 8 (0.3-3 mg/kg), produced dose-dependent relief of tactile allodynia in the ipsilateral hindpaw of CCI-rats, whereas vehicle did not. At the highest dose tested, there was a rapid onset of anti-allodynia with mean peak responses attained at ˜0.5 h post-dosing and with a relatively long duration of action (>3 h). Intravenous administration of a single bolus dose (1 mg/kg) of the endogenous μ-opioid peptide, endomorphin-1 (Endo1), otherwise known as Endo1 in this study, produced a brief period (˜1.25 h) of significant anti-allodynia post-dosing in the ipsilateral hindpaw of adult male CCI-rats, with the mean peak response observed at ˜0.75 h. Comparison of the dose-normalised extent and duration of the anti-allodynic responses (AUC values) of Endo1 and Compound 8 showed that the potency of Compound 8 was significantly (p<0.05) greater than that of Endo1 in CCI-rats.

When Endo1 and Compound 8 were administered intravenously, there was no significant antinociception in the contralateral hindpaw of CCI-rats. The difference in activity between the ipsilateral paw and the contralateral paw suggests an unexpected mechanism of action for Compound 8 when compared with opioids such as morphine. Moreover, the lack of antinociceptive effect in the contralateral hindpaw suggests that Compound 8 targets the nociceptive pathway associated with injury rather than augmenting the descending inhibitory system in a manner similar to morphine. A novel activity may result in a different side effect profile when compared, for example, with morphine. The novel activity may result in a difference in tolerance, when compared, for example, with morphine, it may result in a decrease in tolerance.

Single bolus s.c. doses of Compound 8 (0.1-10 mg/kg), produced dose-dependent relief of tactile allodynia in the ipsilateral hindpaw of CCI-rats, whereas vehicle did not. At the highest dose tested, there was a rapid onset of anti-allodynia with mean peak responses attained at ˜0.5 h post-dosing. Single bolus s.c. doses of Compound 8 (0.3-3 mg/kg), did not produce dose-dependent anti-nociceptive pain relief in the contralateral hindpaw of CCI-rats. These results indicate that compound 8 is capable of crossing the s.c. layer, and providing anti-allodynic pain relief.

### Example 16

### Gut Motility Studies

Each dose of Compound 8, morphine and vehicle was administered to naïve rats as a 100 μL i.v. bolus injection via an indwelling cannula surgically implanted into the jugular vein at least 12 h prior to dose administration. Rats were anaesthetised with 3% isoflurane: 97% oxygen inhalational anaesthetic. A 1 cm incision was made on the right ventral side of the trachea. Using blunt dissection, the jugular vein was exposed and a small incision made to allow cannula insertion. The cannula was inserted into the vein, its integrity assessed, and tied into place using sterile sutures. The wound was sutured, the cannula was exteriorised at the back of the neck and secured using a metal spring. The rats were kept warm during surgical recovery. Animals were housed singly for the remainder of the experimentation period. Rats were inspected regularly from the time of surgery with respect to exploring behaviour, body weight and water intake. No adverse signs from the surgery were observed.

Charcoal meal was prepared by mixing 10% charcoal and 5% arabic gum in distilled, deionised water. Each rat received a single oral gavage of charcoal meal. Thirty minutes later the animal was euthanised using CO2 asphyxiation and the intestine was removed and measured. The distance from the pyloric sphincter to the caudal end of the charcoal and the total distance of the small intestine to the distal part of the ileum were measured in cm. Gastrointestinal motility was determined as the distance traveled by the charcoal relative to the total length of the small intestine, expressed as a percentage.

Each rat received a single bolus i.v. dose of Compound 8, morphine or vehicle. Animals received either vehicle (n=5), morphine at 1 mg/kg (n=3), 2 mg/kg (n=5), 10 mg/kg (n=5) and 30 mg/kg (n=2) or Compound 8 at 0.3 mg/kg (n=3), 1 mg/kg (n=5), 3 mg/kg (n=4), and 10 mg/kg (n=3). Thirty minutes after i.v. dosing an oral gavage of charcoal meal was administered under light (50%:50% CO2:O2) anaesthesia. Thirty minutes later the animal was euthanised via CO2 asphyxiation and the intestine was removed and the distance moved by the charcoal meal was measured.

The intestine was measured for distance of charcoal meal traveled in the small intestine and total length of small intestine. The % distance of charcoal transit was assessed relative to the total length of the small intestine. % inhibition of gastrointestinal motility by morphine or Compound 8 was calculated by the following equation: [(average % charcoal transit in vehicle rats)−(% distance charcoal transit)×100]/(average % charcoal transit in vehicle rats).

The mean(±SEM) total length of the small intestine was 93.2 (±0.9) cm. The mean(±SEM) distance traveled by charcoal meal at 30 min was 44 (±0.8) cm. Thus, the mean(±SEM) % distance traveled by the charcoal meal was 47 (±0.8) % (FIG. 1). Single intravenous bolus doses of morphine at 1 and 2 mg/kg did not significantly (p>0.05) inhibit gastrointestinal motility; however at doses of 10 and 30 mg/kg significant (p<0.05) inhibition of gastrointestinal transit occurred. Compound 8 inhibited gastrointestinal motility at all doses assessed (0.3, 1, 3 and 10 mg/kg) in a dose-dependent manner.

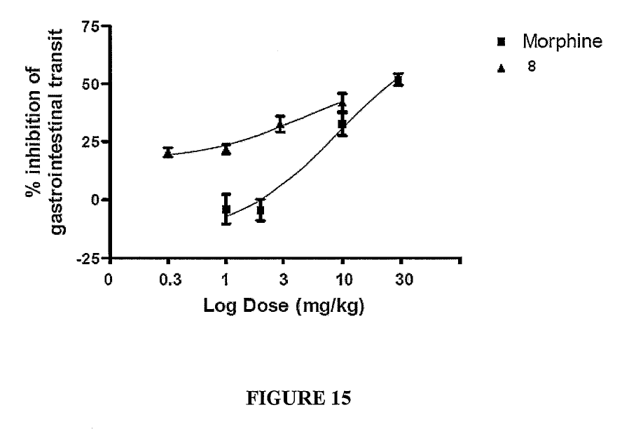

FIG. 15 demonstrates the log dose % inhibition response curve for single bolus i.v. doses of morphine and Compound 8. The slope of the response curve appears to be steeper for morphine than Compound 8. The % inhibition of gastrointestinal transit did not differ significantly (p>0.05) between i.v. morphine and Compound 8 at the 10 mg/kg dose.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A derivative of an endomorphin, or of an endomorphin analog, comprising at least one moiety selected from a lipid moiety, a cyclitol moiety and a saccharide moiety.

2. The derivative of an endomorphin, or of an endomorphin analog, according to claim 1 which comprises a moiety represented by formula I:
Q¹-P¹-Q³-Q⁴ formula I
wherein:
Q¹ is selected from an optionally substituted phenolic amino acid residue;
P¹ is an amino acid residue or is a linker moiety which is further substituted with a cyclitol, saccharide moiety and/or a lipidic group;
Q³ is selected from an optionally substituted aromatic amino acid residue; and
Q⁴ is selected from an optionally substituted aromatic amino acid residue;
with the proviso that at least one lipidic, cyclitol or saccharide moiety is conjugated to the compound comprising the moiety represented by formula I.

3. The derivative of an endomorphin, or of an endomorphin analog, according to claim 1 which is represented by formula II:
L¹-Q¹-P¹-Q³-Q⁴-L²-A¹ formula II
wherein L¹, L² and A¹ may each be independently present or absent;
and wherein:
Q¹ is selected from an optionally substituted phenolic amino acid residue;
P¹ is an amino acid residue or is a linker moiety which is further substituted with a cyclitol, saccharide moiety and/or a lipidic group;
Q³ is selected from an optionally substituted aromatic amino acid residue;
Q⁴ is selected from an optionally substituted aromatic amino acid residue;
A¹ is selected from an amine, amide or amide mimetic; and
L¹ and L² are moieties represented by formula III:
Y¹—Y²—Y³—Y⁴ formula III
wherein:
each of Y¹, Y², Y³ and Y⁴ is independently absent or present and are independently selected from:
an amino acid moiety which is further substituted with a lipidic, cyclitol or saccharide moiety; and
a linker moiety which may be further substituted with a lipidic, cyclitol or saccharide moiety;
with the provisos: that when L¹ is present, at least one of Y¹, Y², Y³ and Y⁴ is present in L¹; and that when L² is present, at least one of Y¹, Y², Y³ and Y⁴ is present in L²;
and wherein at least one of P¹, Y¹, Y², Y³ or Y⁴ is an amino acid or linker moiety which is further substituted with a lipidic, cyclitol or saccharide moiety.

4. The derivative of an endomorphin, or of an endomorphin analog, of claim 2 wherein when present each linker moiety is represented by formula IV:
—W-Alk¹-T-Alk²- formula IV
wherein each of W, Alk¹, T and Alk² may be present or absent, provided that at least one of W, Alk¹, T or Alk² is present and wherein:
W is selected from —N(R^{G})—, —NH(CO)—, —C(O)NH—, —S— and —O—, wherein R^{G} is hydrogen, optionally substituted C₁₋₆alkyl, optionally substituted arylC₁₋₄alkyl, optionally substituted aryl or optionally substituted heteroaryl;
Alk¹ is selected from an optionally substituted C₁₋₄alkylene, optionally substituted C₂₋₅alkenylene, optionally substituted C₂₋₅alkynylene, optionally substituted arylene, optionally substituted heteroarylene and an optionally substituted C₁₋₄alkylenearyl,
with the proviso that both W and T are not simultaneously present when Alk¹ is absent;
T is selected from —NH—, —O—, —S—, —NHC(O)—, —C(O)NH—, —NHSO₂—, —C(R^{G})═N—NH—, —NHC(O)NH—, —NHC(S)NH—, —C(R^{G})═N— and —N═C(R^{G})—; and
Alk² is selected from an optionally substituted C₁₋₄alkylene, optionally substituted C₂₋₅alkenylene, optionally substituted C₂₋₅alkynylene, optionally substituted arylene, optionally substituted heteroarylene and an optionally substituted C₁₋₄alkylenearyl.

5. The derivative of an endomorphin, or of an endomorphin analog, according to claim 2 wherein P¹ is an alpha- or beta-aliphatic or aromatic amino acid residue.

6. The derivative of an endomorphin, or of an endomorphin analog, according to claim 2 wherein P¹ is an optionally substituted heterocyclic amino acid residue.

7. The derivative of an endomorphin, or of an endomorphin analog, according to claim 2 wherein P¹ is an optionally substituted alpha- or beta-proline residue.

8. The derivative of an endomorphin, or of an endomorphin analog, according to claim 2 wherein P¹ is an amino acid residue which is further substituted with a lipidic, cyclitol or saccharide moiety.

9. The derivative of an endomorphin, or of an endomorphin analog, according to claim 2 wherein the side chains of the amino acid residues represented by Q³ and Q⁴ are each independently selected from an optionally substituted arylC₁₋₄alkyl group and an optionally substituted 3-indolylmethyl group.

10. The derivative of an endomorphin, or of an endomorphin analog, according to claim 1 which is represented by formula V: 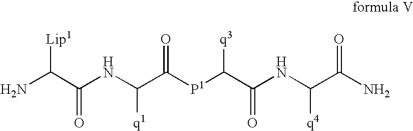
wherein:
P¹ is an amino acid residue or is a linker moiety which is further substituted with a cyclitol, saccharide moiety and/or a lipidic group;
Lip¹ is a lipidic group;
q¹ is a phenolic side chain; and
q³ and q⁴ are aromatic side chains.

11. The derivative of an endomorphin, or of an endomorphin analog, according to claim 1 which is represented by formula VI: 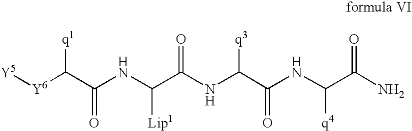
wherein:
Lip¹ is a lipidic group;
q¹ is a phenolic side chain; and
q³ and q⁴ are aromatic side chains;
Y⁵ is an amino acid moiety or a linking group further substituted with a cyclitol or saccharide group; and
Y⁶ is a linker moiety.

12. The derivative of an endomorphin, or of an endomorphin analog, according to claim 1 which is represented by formula VII: 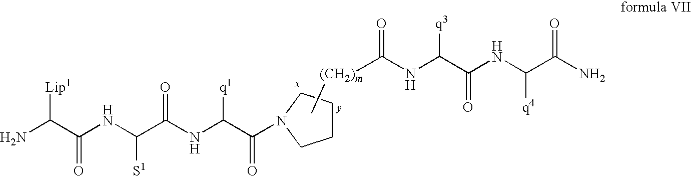
wherein:
Lip¹ is a lipidic group;
q¹ is a phenolic side chain; and
q³ and q⁴ are aromatic side chains;
S¹ is a cyclitol or saccharide group
m is an integer from 0 to 2; and
x and y refer to points of substitution on the pyrrolidine ring.

13. The derivative of an endomorphin, or of an endomorphin analog, according to claim 1 which is represented by formula VIII: 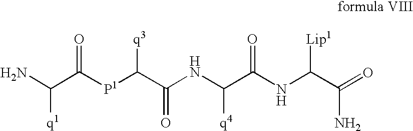
wherein:
Lip¹ is a lipidic group;
q¹ is a phenolic side chain; and
q³ and q⁴ are aromatic side chains.

14. The derivative of an endomorphin, or of an endomorphin analog, according to claim 1 which is represented by formula IX: 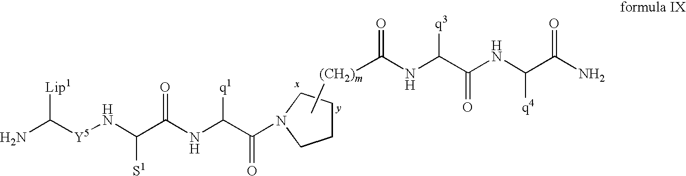
wherein:
Lip¹ is a lipidic group;
q¹ is a phenolic side chain; and
q³ and q⁴ are aromatic side chains;
S¹ is a cyclitol or saccharide group;
m is an integer from 0 to 2;
x and y refer to points of substitution on the pyrrolidine ring; and
Y⁵ is a linker moiety.

15. The derivative of an endomorphin, or of an endomorphin analog, according to claim 1 which is represented by formula X: 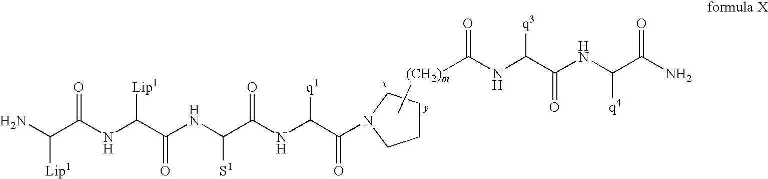
wherein:
each Lip¹ may be the same or different and is a lipidic group;
q¹ is a phenolic side chain;
q³ and q⁴ are aromatic side chains;
S¹ is a cyclitol or saccharide group;
m is an integer from 0 to 2; and
x and y refer to points of substitution on the pyrrolidine ring.

16. The derivative of an endomorphin, or of an endomorphin analog, according to claim 1 which is represented by formula XI: 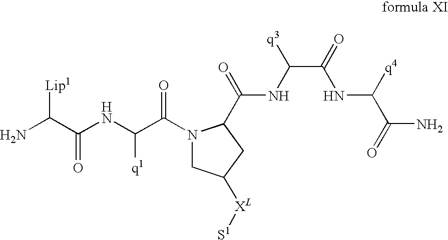
wherein:
Lip¹ is a lipidic group;
q¹ is a phenolic side chain; and
q³ and q⁴ are aromatic side chains;
S¹ is a cyclitol or saccharide group; and
X^{L} is a linker moiety represented by Formula IV;
—W-Alk¹-T-Alk²- formula IV
wherein each of W, Alk¹, T and Alk² may be present or absent, provided that at least one of W, Alk¹, T or Alk² is present and wherein:
W is selected from —N(R^{G})—, —NH(CO)—, —C(O)NH—, —S— and —O—, wherein R^{G} is hydrogen, optionally substituted C₁₋₆alkyl, optionally substituted arylC₁₋₄alkyl, optionally substituted aryl or optionally substituted heteroaryl;
Alk¹ is selected from an optionally substituted C₁₋₄alkylene, optionally substituted C₂₋₅alkenylene, optionally substituted C₂₋₅alkynylene, optionally substituted arylene, optionally substituted heteroarylene and an optionally substituted C₁₋₄alkylenearyl;
with the proviso that both W and T are not simultaneously present when Alk¹ is absent;
T is selected from —NH—, —O—, —S—, —NHC(O)—, —C(O)NH—, —NHSO₂—, —C(R^{G})═N—NH—, —NHC(O)NH—, —NHC(S)NH—, —C(R^{G})═N— and —N═C(R^{G})—; and
Alk² is selected from an optionally substituted C₁₋₄alkylene, optionally substituted C₂₋₅alkenylene, optionally substituted C₂₋₅alkynylene, optionally substituted arylene, optionally substituted heteroarylene and an optionally substituted C₁₋₄alkylenearyl.

17. The derivative of an endomorphin, or of an endomorphin analog, according to claim 1 which is represented by formula XII: 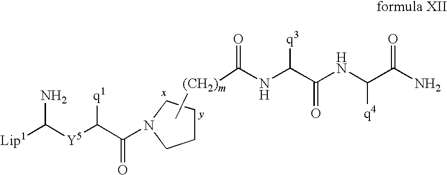
wherein
Lip¹ is a lipidic group;
q¹ is a phenolic side chain; and
q³ and q⁴ are aromatic side chains;
m is an integer from 0 to 2;
x and y refer to points of substitution on the pyrrolidine ring; and
Y⁵ is a linking moiety.

18. The derivative of an endomorphin, or of an endomorphin analog, according to claim 1 which is represented by formula XIII: 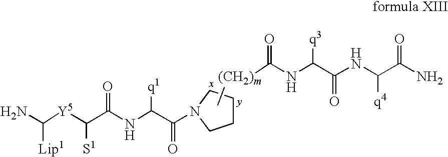
wherein:
Lip¹ is a lipidic group;
q¹ is a phenolic side chain;
q³ and q⁴ are aromatic side chains;
S¹ is a cyclitol or saccharide group;
m is an integer from 0 to 2;
x and y refer to points of substitution on the pyrrolidine ring; and
Y⁵ is a linking moiety.

19. The derivative of an endomorphin, or of an endomorphin analog, according to claim 1 which is represented by formula XIV: 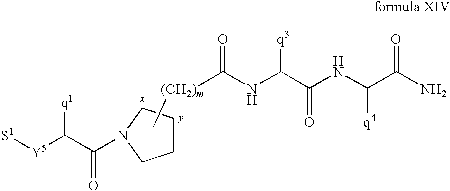
wherein:
q¹ is a phenolic side chain;
q³ and q⁴ are aromatic side chains;
S¹ is a cyclitol or saccharide group
m is an integer from 0 to 2;
x and y refer to points of substitution on the pyrrolidine ring; and
Y⁵ is a linking moiety.

20. The derivative of an endomorphin, or of an endomorphin analog, according to claim 1 which is represented by formula XV: 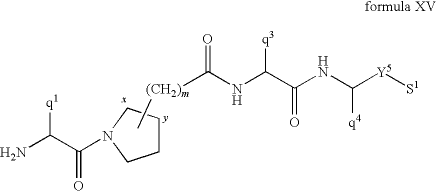
wherein:
q¹ is a phenolic side chain;
q³ and q⁴ are aromatic side chains;
S¹ is a cyclitol or saccharide group;
m is an integer from 0 to 2;
x and y refer to points of substitution on the pyrrolidine ring; and
Y⁵ is a linking moiety.

21. The derivative of an endomorphin, or of an endomorphin analog, according to claim 1 which is represented by formula XVI: 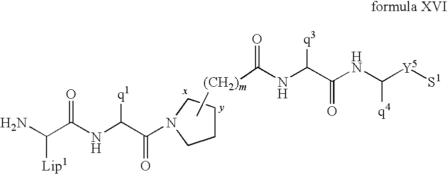
wherein:
Lip¹ is a lipidic group;
q¹ is a phenolic side chain;
q³ and q⁴ are aromatic side chains;
S¹ is a cyclitol or saccharide group;
m is an integer from 0 to 2;
x and y refer to points of substitution on the pyrrolidine ring; and
Y⁵ is a linking moiety.

22. The derivative of an endomorphin, or of an endomorphin analog, according to claim 1 which is represented by formula XVII: 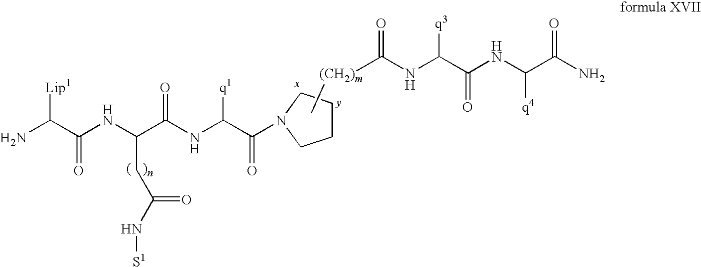
wherein:
Lip¹ is a lipidic group;
q¹ is a phenolic side chain;
q³ and q⁴ are aromatic side chains;
S¹ is a cyclitol or saccharide group;
m is an integer from 0 to 2;
x and y refer to points of substitution on the pyrrolidine ring; and
n is an integer from 1 to 4.

23. The derivative of an endomorphin, or of an endomorphin analog, according to claim 1 which is represented by formula XVIII, 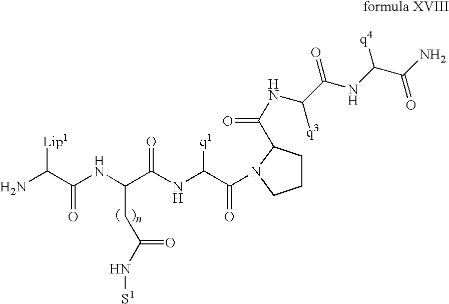
wherein:
Lip¹ is C₆₋₁₀alkyl;
n is an integer from 1 to 4;
S¹ is a disaccharide;
q¹ is selected from 4-hydroxybenzyl and 2,6-di-C₁₋₄alkyl-4-hydroxybenzyl;
q³ is selected from benzyl, α-methylbenzyl and 3-indolylmethyl; and
q⁴ is selected from benzyl and α-methylbenzyl.

24. A compound according to claim 23 wherein Lip¹ is octanyl and the disaccharide S¹ is selected from lactose, melibiose, cellobiose, isomaltose, maltose, allolactose and gentobiose.

25. A compound according to claim 1, wherein the derivative of an endomorphin, or of an endomorphin analog, is fused with a heterologous polypeptide.

26. The fused compound according to claim 25 wherein the heterologous polypeptide is biologically active or has a carrier function.

27. The derivative of an endomorphin, or of an endomorphin analog, according to claim 1 which is represented by formula XIX: 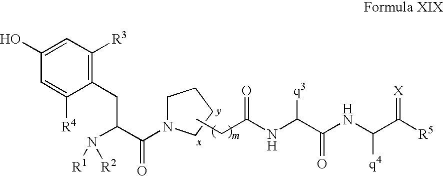
wherein:
m is 0 or 1;
x and y represent points of substitution on the pyrrolidine ring;
q³ and q⁴ are each independently selected from an aryl, arylC₁₋₄alkyl, heteroaryl and
heteroarylC₁₋₄alkyl group;
R¹ and R² are each independently selected from hydrogen and —C(O)R⁶, or R¹ and R²
taken together form ═CHR⁷;
wherein:
R⁶ is selected from C₅₋₂₀alkyl, C₅₋₂₀alkenyl, C₅₋₂₀alkynyl, —CH(NH₂)(C₄₋₁₉alkyl), —CH(NH₂)(C₄₋₁₉alkenyl), —CH(NH₂)(C₄₋₁₉alkynyl), —CH(NH₂)(aminoC₁₋₆alkyl), —CH(NH₂)(aminoC₂₋₆alkenyl), —CH(NH₂)(aminoC₂₋₆alkynyl), —CH(aminoC₁₋₆alkyl)NHC(O)(CH₂)ₜC(O)NHS¹, —CH(carboxyC₁₋₆alkyONHC(O)CH(NH₂)C₅₋₂₀alkyl, —CH(carboxyC₁₋₆alkyl)NHC(O)CH(NH₂)C₅₋₂₀alkenyl, —CH(carboxyC₁₋₆alkyl)NHC(O)CH(NH₂)C₅₋₂₀alkynyl, —CH(R⁸)(CH₂)_{q}C(O)NHS¹, —CH(NHC(O)R¹¹)(CH₂)ᵤNHC(O)R¹⁰, —CH(C₅₋₂₀alkyl)NHC(O)(CH₂)ₜC(O)NHS¹, —CH(C₅₋₂₀alkenyl)NHC(O)(CH₂)ₜC(O)NHS¹, and —CH(C₅₋₂₀alkynyl)NHC(O)(CH₂)ₜC(O)NHS¹,
wherein:
q is 1 to 4;
u is 1 to 6;
R⁸ is selected from hydrogen, —NH₂, —NHC(O)CH(NH₂)(C₅₋₂₀alkyl), —NHC(O)CH(NH₂)(C₅₋₂₀alkenyl), and —NHC(O)CH(NH₂)(C₅₋₂₀alkynyl);
R¹⁰ is selected from C₅₋₂₀alkyl, C₅₋₂₀alkenyl, C₅₋₂₀alkynyl, —CH(NH₂)(C₄₋₁₉alkyl), —CH(NH₂)(C₄₋₁₉alkenyl), —CH(NH₂) (C₄₋₉alkynyl), and —(CH₂)ᵥC(O)NHS⁴,
wherein:
v is 2 to 4;
R¹¹ is selected from —(CH₂)₁C(O)NHS¹, C₅₋₂₀alkyl, C₅₋₂₀alkenyl, C₅₋₂₀alkynyl, —CH(NH₂)(C₄₋₁₉alkyl), —CH(NH₂)(C₄₋₁₉alkenyl), and —CH(NH₂)(C₄₋₁₉alkynyl);
t is 2 to 4; and
S¹ and S⁴ are each independently a cyclitol or saccharide;
R⁷ is selected from C₅₋₂₀alkyl, C₅₋₂₀alkenyl, C₅₋₂₀alkynyl, —CH(NH₂)(C₄₋₁₉alkyl), —CH(NH₂)(C₄₋₁₉alkenyl), —CH(NH₂)(C₄₋₁₉alkynyl) and —CH(R⁸)(CH₂)ᵣC(O)NHS²;
wherein:
r is 1 or 2; and
S² is a cyclitol or saccharide;
R³ and R⁴ are each independently selected from hydrogen, C₁₋₄alkyl and C₂₋₄alkenyl;
X is selected from O and N—R⁹;
wherein:
R⁹ is selected from hydrogen, C₅₋₂₀alkyl, C₅₋₂₀alkenyl, C₅₋₂₀alkynyl, —CH(C(O)NH₂)(C₄₋₁₉alkyl), —CH(C(O)NH₂)(C₄₋₁₉alkenyl), and —CH(C(O)NH₂)(C₄₋₁₉alkynyl);
R⁵ is selected from hydrogen, —NH₂, —NHS³, —NHCH(C(O)NH₂)(C₅₋₂₀alkyl), —NHCH(C(O)NH₂)(C₅₋₂₀alkenyl), —NHCH(C(O)NH₂)(C₅₋₂₀alkynyl), —NH(CO)CH(carboxyC₁₋₆alkyl)NHCH(C(O)NH₂)(C₅₋₂₀alkyl), —NH(CO)CH(carboxyC₁₋₆alkyl)NHCH(C(O)NH₂)(C₅₋₂₀alkenyl), and —NH(CO)CH(carboxyC₁₋₆alkyl)NHCH(C(O)NH₂)(C₅₋₂₀alkynyl);
wherein:
S³ is a cyclitol or saccharide,
wherein at least one of R¹, R², X and R⁵ represents a substituent comprising a cyclitol, a saccharide or an alkyl, alkenyl or alkynyl group comprising 4 or more carbon atoms.

28. The derivative of an endomorphin, or of an endomorphin analog, according to claim 27 wherein q³ is selected from the group consisting of: 3-indolylmethyl, α-methylbenzyl, benzyl, 1-naphthyl, 2-naphthyl, 2-phenylphenyl, 3-phenylphenyl and 4-phenylphenyl.

29. The derivative of an endomorphin, or of an endomorphin analog, according to claim 27 wherein q⁴ is selected from the group consisting of: benzyl, α-methylbenzyl, 2-methylphenyl, 3-methylphenyl, 4-methylphenyl, 2,3-dimethylphenyl, 2,4-dimethylphenyl, 3,4-dimethylphenyl, 1-naphthyl, 2-naphthyl 2-phenylphenyl, 3-phenylphenyl and 4-phenylphenyl.

30. The derivative of an endomorphin, or of an endomorphin analog, according to claim 27 wherein R² is hydrogen and R¹ is selected from the group consisting of: —C(O)CH(NH₂)(C₄₋₁₉alkyl), —C(O)CH(aminoC₃₋₅alkyl)NHC(O)(CH₂)₂C(O)NHS¹, —C(O)CH(NHC(O)(CH₂)₂C(O)NHS¹)(CH₂)₄NHC(O)CH(NH₂)(C₄₋₁₉alkyl), —C(O)CH(NHC(O)(CH₂)₂C(O)NHS¹(CH₂)₄NHC(O)(CH₂)₂C(O)NHS¹, —C(O)CH(NHC(O)C₅₋₂₀alkyl)(CH₂)₄NHC(O)(CH₂)₂C(O)NHS¹, —C(O)CH(C₅₋₂₀alkyl)NHC(O)(CH₂)₂C(O)NHS¹, —C(O)CH(carboxyC₁₋₆alkyl)NHC(O)CH(NH₂)(C₅₋₂₀alkyl), —C(O)CH₂(CH₂)_{q}C(O)NHS¹, —C(O)CH(NH₂)(CH₂)_{q}C(O)NHS¹ and —C(O)CH(NHC(O)CH(NH₂)(C₅₋₂₀alkyl))(CH₂)_{q}C(O)NHS¹,
wherein q is 1 to 4, and S¹ is a saccharide or cyclitol.

31. The derivative of an endomorphin, or of an endomorphin analog, according to claim 30 wherein R² is hydrogen and R¹ is selected from the group consisting of:
—C(O)CH(NH²)(_{C5-18}alkyl),
—C(O)CH(aminobutyl)NHC(O)(CH₂)₂C(O)NHS¹,
—C(O)CH(NHC(O)(CH₂)₂C(O)NHS¹(CH₂)₄NHC(O)CH(NH₂)(C₅₋₁₂alkyl),
—C(O)CH(NHC(O)(CH₂)₂C(O)NHS¹)(CH₂)₄NHC(O)(CH₂)₂C(O)NHS¹,
—C(O)CH(NHC(O)C₅₋₁₂alkyl)(CH₂)₄NHC(O)(CH₂)₂C(O)NHS¹,
—C(O)CH(C₅₋₁₂alkyl)NHC(O)(CH₂)₂C(O)NHS¹,
—C(O)CH(carboxyC₁₋₂alkyl)NHC(O)CH(NH₂)(C₅₋₁₂alkyl),
—C(O)CH₂(CH₂)_{q}C(O)NHS¹, —C(O)CH(NH₂)(CH₂)_{g}C(O)NHS¹ and
—C(O)CH(NHC(O)CH(NH₂)(C₅₋₁₂alkyl))(CH₂)_{q}C(O)NHS¹,
wherein q is 1 to 3, and S¹ is a saccharide or cyclitol.

32. The derivative of an endomorphin, or of an endomorphin analog, according to claim 27 wherein R¹ and R² together form ═CHCH(NH₂)(C₄₋₁₉alkyl).

33. The derivative of an endomorphin, or of an endomorphin analog, according to claim 32 wherein R¹ and R² together form ═CHCH(NH₂)(C₅₋₁₂alkyl).

34. The derivative of an endomorphin, or of an endomorphin analog, according to claim 27 wherein R¹ and R² are each hydrogen.

35. The derivative of an endomorphin, or of an endomorphin analog, according to claim 27 wherein R³ and R⁴ are each independently selected from hydrogen and C₁₋₂alkyl.

36. The derivative of an endomorphin, or of an endomorphin analog, according to claim 27 wherein X is selected from oxygen and ═NCH(C(O)NH₂)(C₅₋₁₂alkyl).

37. The derivative of an endomorphin, or of an endomorphin analog, according to claim 27 wherein R⁵ is selected from the group consisting of: hydrogen, —NH₂, —NHS³, —NHCH(C(O)NH₂)(C₅₋₁₂alkyl) and —NH(CO)CH(carboxyC₁₋₂alkyl)NHCH(C(O)NH₂)(C₅₋₁₂alkyl), wherein S³ is a saccharide or cyclitol.

38. The derivative of an endomorphin, or of an endomorphin analog, according to claim 27 wherein, when present, S¹, S² and S³ are each independently selected from the group consisting of: lactosyl, glucopyranosyl, mannopyranosyl, galactopyranosyl, 2-deoxy-2-acetamido-glucopyranosyl, 2-deoxy-2-acetamido-galactopyranosyl, maltosyl, glucoronyl, galacturonyl, melibiosyl, cellobiosyl, isomaltosyl, allolactosyl and gentobiosyl.

39. A derivative of an endomorphin, or of an endomorphin analog, according to claim 27 which is selected from the compounds as herein described in Table 2.

40. A derivative of an endomorphin, or of an endomorphin analog, according to claim 1 which is selected from:

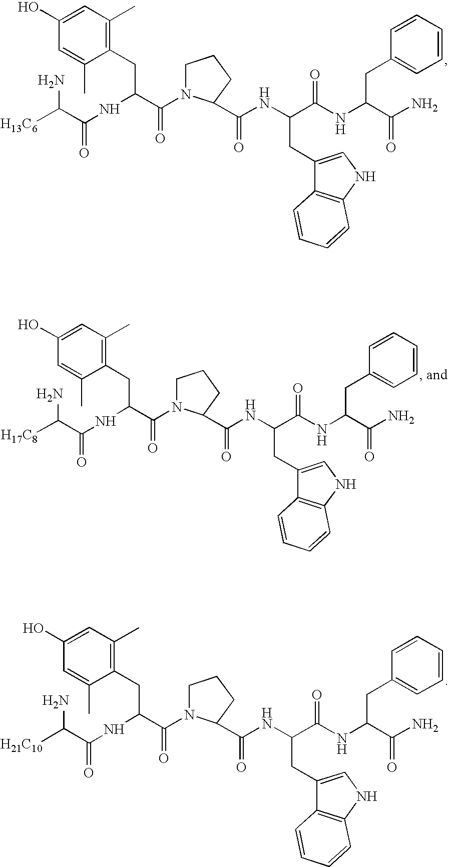

41. A derivative of an endomorphin, or of an endomorphin analog, according to claim 1 which is represented by the following structure:

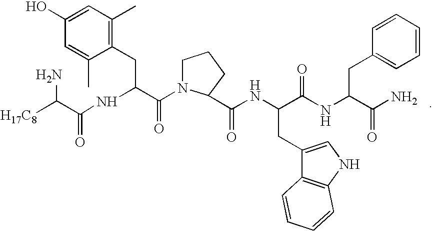

42. A pharmaceutical composition comprising a compound according to claim 1, or the salt, solvate or hydrate thereof, and at least one pharmaceutical excipient.

43. (canceled)

44. A method of prevention, treatment, reversal and/or symptomatic relief of pain comprising administering to a subject in need thereof a compound according to claim 1.

45. (canceled)
